(19) United States
(12) Patent Application Publication
(10) Pub. No.: US 2025/0049838 A1
(43) Pub. Date: Feb. 13, 2025

(54) Antisense Compounds Targeting Genes Associated with Cystic Fibrosis

(71) Applicant: Rosalind Franklin University of Medicine and Science, North Chicago, IL (US)

(72) Inventors: Michelle L. HASTINGS, North Chicago, IL (US); Wren E. MICHAELS, North Chicago, IL (US)

(21) Appl. No.: 18/933,256

(22) Filed: Oct. 31, 2024

Related U.S. Application Data
(63) Continuation-in-part of application No. 17/472,887, filed on Sep. 13, 2021.
Application No. 17/472,887 is a continuation of application No. 16/730,517, filed on Dec. 30, 2019, now Pat. No. 11,116,785.
Application No. 16/730,517 is a continuation of application No. 15/835,698, filed on Dec. 8, 2017, now Pat. No. 10,525,076.
Application No. 15/835,698 is a continuation-in-part of application No. 15/045,999, filed on Feb. 17, 2016, now Pat. No. 9,840,709.
(60) Provisional application No. 62/118,794, filed on Feb. 20, 2015.
(60) Provisional application No. 63/148,682, filed on Feb. 12, 2021.
(60) Provisional application No. 63/594,941, filed on Oct. 31, 2023.

(51) Int. Cl. A61K 31/712; C12N 15/113

(52) U.S. Cl. CPC A61K 31/712; C12N 15/1138; A01K 2267/0306; C12N 2310/11; C12N 2310/321; C12N 2310/3233; C12N 2320/33

(57) ABSTRACT

The present disclosure relates generally to compounds comprising oligonucleotides complementary to a cystic fibrosis transmembrane conductance regulator (CFTR) RNA transcript. Certain such compounds are useful for hybridizing to a CFTR RNA transcript, including but not limited to a CFTR RNA transcript in a cell. In certain embodiments, such hybridization results in modulation of splicing and/or expression of the CFTR transcript. In certain embodiments, such compounds are used to treat one or more symptoms associated with Cystic Fibrosis.

### CROSS REFERENCE TO RELATED APPLICATIONS

This application is a continuation-in-part of U.S. application Ser. No. 17/472,887, filed Sep. 13, 2021, which is a continuation-in-part of U.S. application Ser. No. 16/730,517, filed Dec. 30, 2019 (now U.S. Pat. No. 11,116,785, issued Sep. 14, 2021), which is a continuation of U.S. application Ser. No. 15/835,698, filed Dec. 8, 2017 (now U.S. Pat. No. 10,525,076, issued Jan. 7, 2020), which is a continuation-in-part of U.S. application Ser. No. 15/045,999, filed Feb. 17, 2016 (now U.S. Pat. No. 9,840,709, issued Dec. 12, 2017), which is a non-provisional application of U.S. Provisional Application No. 62/118,794, filed Feb. 20, 2015, the disclosures of which each of which are incorporated by reference in their entirety. U.S. application Ser. No. 17/472,887 claims the benefit of priority to U.S. Provisional Application No. 63/148,682, filed Feb. 12, 2021, the disclosure of which is incorporated by reference in its entirety. This application also claims the benefit of priority to U.S. Provisional Application No. 63/594,941, filed Oct. 31, 2023, the disclosure of which is incorporated by reference in its entirety.

### SEQUENCE LISTING

A computer readable form of the Sequence Listing is filed with this application by electronic submission and is incorporated into this application by reference in its entirety. The Sequence Listing is contained in the XML file created on Oct. 31, 2024, having the file name “15-311-US-CIP3_Sequence-Listing.xml” and is 704,427 bytes in size.

### FIELD OF THE DISCLOSURE

The present disclosure relates generally to compounds comprising oligonucleotides complementary to a cystic fibrosis transmembrane conductance regulator (CFTR) RNA transcript. Certain such compounds are useful for hybridizing to a CFTR transcript, including but not limited to a CFTR RNA transcript in a cell. In certain embodiments, such hybridization results in modulation of expression and/or splicing of the CFTR transcript. In certain embodiments, such compounds are used to treat one or more symptoms associated with Cystic Fibrosis.

### BACKGROUND OF THE DISCLOSURE

Cystic fibrosis (CF), also known as mucoviscidosis, is a genetic disorder that affects mostly the lungs, but also the pancreas, liver, kidneys, and intestine. Long-term issues include difficulty breathing and coughing up mucus as a result of frequent lung infections. Other signs and symptoms include sinus infections, poor growth, fatty stool, clubbing of the fingers and toes, and infertility in males among others. Different people may have different degrees of symptoms.

CF is inherited in an autosomal recessive manner. It is caused by the presence of mutations in both copies of the gene for the cystic fibrosis transmembrane conductance regulator (CFTR) protein. Those with a single working copy are carriers and otherwise mostly normal. CFTR is involved in production of sweat, digestive fluids, and mucus. When CFTR is not functional, secretions, which are usually thin, instead become thick. The condition is diagnosed by a sweat test and genetic testing. Screening of infants at birth takes place in some areas of the world.

There is no cure for cystic fibrosis. Lung infections are treated with antibiotics which may be given intravenously, inhaled, or by mouth. Sometimes the antibiotic azithromycin is used long term. Inhaled hypertonic saline and salbutamol may also be useful. Lung transplantation may be an option if lung function continues to worsen. Pancreatic enzyme replacement and fat-soluble vitamin supplementation are important, especially in the young. The average life expectancy is between 42 and 50 years in the developed world. While CF is a multi-organ disease, lung problems are the dominant cause of morbidity and mortality. Other CF symptoms include pancreatic insufficiency, intestinal obstruction, elevated electrolyte levels in sweat (the basis of the most common diagnostic test), and male infertility. CF is most common among people of Northern European ancestry and affects about one out of every 2,500 to 4,000 newborns. About one in 25 people are carriers. While treatments for Cystic Fibrosis are available, more effective therapies are needed.

### SUMMARY OF THE DISCLOSURE

The present disclosure relates to general compounds and methods to treat cystic fibrosis in subjects using antisense oligonucleotides (ASOs) that induce specific pre-mRNA splicing events in CFTR gene transcripts that result in mRNAs that code for proteins that fully or partially restore the function of CFTR (i.e., resulting in increased levels of correctly localized CFTR protein at the plasma membrane and with increased function).

In one aspect, the disclosure provides a composition comprising two or more modified oligonucleotides, wherein each of the one or more modified oligonucleotides consists of 8 to 30 linked nucleosides, wherein the nucleobase sequence of each of the one or more modified oligonucleotides is at least 80%, complementary to an equal-length portion of a target region of a cystic fibrosis transmembrane conductance regulator (CFTR) transcript, wherein the target region is within: (a) nucleobase 198,888 and nucleobase 199,094 of SEQ ID NO: 130; or (b) nucleobase 199,705 and nucleobase 199,796 of SEQ ID NO: 130. In some embodiments, the target region is within nucleobase 198,888 and nucleobase 199,796 of SEQ ID NO: 130, and each of the one or more modified oligonucleotides is selected from the group consisting of SEQ ID NOs: 174-181. In certain embodiments, the target region is within nucleobase 198,888 and nucleobase 199,094 of SEQ ID NO: 130, and each of the one or more modified oligonucleotides is selected from the group consisting of SEQ ID NOs: 174-177. In some embodiments, the target region is within nucleobase 199,705 and nucleobase 199,796 of SEQ ID NO: 130, and each of the one or more modified oligonucleotides is selected from the group consisting of SEQ ID NOs: 178-181.

In another aspect, the disclosure provides a compound comprising a modified oligonucleotide having 8 to 30 linked nucleosides having a nucleobase sequence comprising a complementary region, wherein the complementary region comprises at least 8 contiguous nucleobases complementary to an equal-length portion of a target region of a cystic fibrosis transmembrane conductance regulator (CFTR) transcript. In certain embodiments, the target region of the CFTR transcript comprises at least a portion of intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24 or intron 24, exon 25, intron 25, exon 26 or intron 26 of the CFTR transcript. In other embodiments, the nucleobase sequence of the antisense oligonucleotide comprises any one of SEQ ID NOs: 1 to 144, SEQ ID NO:150, or SEQ ID NOs: 174-181.

In another aspect, the disclosure provides a pharmaceutical composition comprising at least one compound as described herein and a pharmaceutically acceptable carrier or diluent.

In yet another aspect, the disclosure provides a method of modulating splicing or expression of a CFTR transcript in a cell comprising contacting the cell with at least one compound as described herein.

The yet another aspect, the disclosure provides a method of treating cystic fibrosis, comprising administering at least one compound as described herein to an animal in need thereof.

In yet another aspect, the disclosure provides a method of modulating splicing or expression of a CFTR transcript in a cell comprising administering to an animal in need thereof a composition comprising one or more modified oligonucleotides, wherein each of the one or more modified oligonucleotides consists of 8 to 30 linked nucleosides, wherein the nucleobase sequence of each of the two or more modified oligonucleotide is at least 80%, complementary to an equal-length portion of a target region of a cystic fibrosis transmembrane conductance regulator (CFTR) transcript. In some embodiments, the target region is within nucleobase 198,888 and nucleobase 199,094 of SEQ ID NO: 130. In some embodiments, the target region is within nucleobase 199,705 and nucleobase 199,796 of SEQ ID NO: 130. In some embodiments, the target region is within nucleobase 198,888 and nucleobase 199,094 of SEQ ID NO: 130, and each of the two or more modified oligonucleotides is selected from the group consisting of SEQ ID NOs: 174-177. In certain embodiments, the target region is within nucleobase 199,705 and nucleobase 199,796 of SEQ ID NO: 130, and each of the two or more modified oligonucleotides is selected from the group consisting of SEQ ID NOs: 178-181. In certain embodiments, the administering step comprises delivering to the animal by inhalation, parenteral injection or infusion, intravenous injection, intrauterine, oral, subcutaneous or intramuscular injection, buccal, transdermal, transmucosal and topical.

The present disclosure provides the following non-limiting numbered embodiments:
Embodiment 1a. A compound comprising a modified oligonucleotide consisting of 8 to 30 linked nucleosides and having a nucleobase sequence comprising a complementary region, wherein the complementary region comprises at least 8 contiguous nucleobases complementary to an equal-length portion of a target region of a cystic fibrosis transmembrane conductance regulator (CFTR) transcript.
Embodiment 1b. A compound comprising two or more modified oligonucleotides, wherein each of the two or more modified oligonucleotides consists of 8 to 30 linked nucleosides, wherein the nucleobase sequence of each of the two or more modified oligonucleotides is at least 80%, complementary to an equal-length portion of a target region of a cystic fibrosis transmembrane conductance regulator (CFTR) transcript.
Embodiment 2a. The compound of embodiment 1, wherein the target region of the CFTR transcript comprises at least a portion of intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24 intron 24, exon 25, intron 25, exon 26 or intron 26 of the CFTR transcript.
Embodiment 2b. The compound of embodiment 1, wherein the target region of the CFTR transcript is within: (a) nucleobase 198,888 and nucleobase 199,094 of SEQ ID NO: 130; or (b) nucleobase 199,705 and nucleobase 199,796 of SEQ ID NO: 130.
Embodiment 3. The compound of embodiment 1, wherein the target region of the CFTR transcript is within about 25 nucleobases upstream or about 25 nucleobases downstream of exon 2, or comprises at least a portion of exon 2 of the CFTR transcript.
Embodiment 4. The compound of embodiment 1, wherein the target region of the CFTR transcript is within about 25 nucleobases upstream or about 25 nucleobases downstream of exon 4, or comprises at least a portion of exon 4 of the CFTR transcript.
Embodiment 5. The compound of embodiment 1, wherein the target region of the CFTR transcript is within about 25 nucleobases upstream or about 25 nucleobases downstream of exon 5, or comprises at least a portion of exon 5 of the CFTR transcript.
Embodiment 6. The compound of embodiment 1, wherein the target region of the CFTR transcript is within about 25 nucleobases upstream or about 25 nucleobases downstream of exon 7, or comprises at least a portion of exon 7 of the CFTR transcript.
Embodiment 7. The compound of embodiment 1, wherein the target region of the CFTR transcript is within about 25 nucleobases upstream or about 25 nucleobases downstream of exon 9, or comprises at least a portion of exon 9 of the CFTR transcript.
Embodiment 8. The compound of embodiment 1, wherein the target region of the CFTR transcript is within about 25 nucleobases upstream or about 25 nucleobases downstream of exon 10, or comprises at least a portion of exon 10 of the CFTR transcript.
Embodiment 9. The compound of embodiment 1, wherein the target region of the CFTR transcript is within about 25 nucleobases upstream or about 25 nucleobases downstream of exon 11, or comprises at least a portion of exon 11 of the CFTR transcript.
Embodiment 10. The compound of embodiment 1, wherein the target region of the CFTR transcript is within about 25 nucleobases upstream or about 25 nucleobases downstream of exon 13, or comprises at least a portion of exon 13 of the CFTR transcript.
Embodiment 11. The compound of embodiment 1, wherein the target region of the CFTR transcript is within about 25 nucleobases upstream or about 25 nucleobases downstream of exon 15, or comprises at least a portion of exon 15 of the CFTR transcript.
Embodiment 12. The compound of embodiment 1, wherein the target region of the CFTR transcript is within about 25 nucleobases upstream or about 25 nucleobases downstream of exon 16, or comprises at least a portion of exon 16 of the CFTR transcript.
Embodiment 13. The compound of embodiment 1, wherein the target region of the CFTR transcript is within about 25 nucleobases upstream or about 25 nucleobases downstream of exon 20, or comprises at least a portion of exon 20 of the CFTR transcript.
Embodiment 14. The compound of embodiment 1, wherein the target region of the CFTR transcript is within about 25 nucleobases upstream or about 25 nucleobases downstream of exon 22, or comprises at least a portion of exon 22 of the CFTR transcript.
Embodiment 15. The compound of embodiment 1, wherein the target region of the CFTR transcript is within about 25 nucleobases upstream or about 25 nucleobases downstream of exon 23, or comprises at least a portion of exon 23 of the CFTR transcript.
Embodiment 16a. The compound of embodiment 1, wherein the target region of the CFTR transcript is within about 25 nucleobases upstream or about 25 nucleobases downstream of exon 24, or comprises at least a portion of exon 24 of the CFTR transcript.
Embodiment 16b. The compound of embodiment 1, wherein the target region of the CFTR transcript is within about 25 nucleobases upstream or about 25 nucleobases downstream of exon 25, or comprises at least a portion of exon 25 of the CFTR transcript.
Embodiment 16c. The compound of embodiment 1, wherein the target region of the CFTR transcript is within about 25 nucleobases upstream or about 25 nucleobases downstream of exon 26, or comprises at least a portion of exon 26 of the CFTR transcript.
Embodiment 17. The compound of any of embodiments 1 to 16, wherein the complementary region of the modified oligonucleotide is at least 80%, at least 85%, at least 90%, at least 95% or at least 100% complementary to the target region.
Embodiment 18. The compound of any of embodiments 1 to 17, wherein the complementary region of the modified oligonucleotide comprises at least 10 contiguous nucleobases.
Embodiment 19. The compound of any of embodiments 1 to 17, wherein the complementary region of the modified oligonucleotide comprises at least 12 contiguous nucleobases.
Embodiment 20. The compound of any of embodiments 1 to 17, wherein the complementary region of the modified oligonucleotide comprises at least 14 contiguous nucleobases.
Embodiment 21. The compound of any of embodiments 1 to 17, wherein the complementary region of the modified oligonucleotide comprises at least 15 contiguous nucleobases.
Embodiment 22. The compound of any of embodiments 1 to 17, wherein the complementary region of the modified oligonucleotide comprises at least 16 contiguous nucleobases.
Embodiment 23. The compound of any of embodiments 1 to 17, wherein the complementary region of the modified oligonucleotide comprises at least 17 contiguous nucleobases.
Embodiment 24. The compound of any of embodiments 1 to 17, wherein the complementary region of the modified oligonucleotide comprises at least 18 contiguous nucleobases.
Embodiment 25. The compound of any of embodiments 1 to 17, wherein the complementary region of the modified oligonucleotide comprises at least 19 contiguous nucleobases.
Embodiment 26. The compound of any of embodiments 1 to 17, wherein the complementary region of the modified oligonucleotide comprises at least 20 contiguous nucleobases.
Embodiment 27. The compound of any of embodiments 1 to 26, wherein the nucleobase sequence of the oligonucleotide is at least 80% complementary to an equal-length region of the CFTR transcript, as measured over the entire length of the oligonucleotide.
Embodiment 28. The compound of any of embodiments 1 to 26, wherein the nucleobase sequence of the oligonucleotide is at least 90% complementary to an equal-length region of the CFTR transcript, as measured over the entire length of the oligonucleotide.
Embodiment 29. The compound of any of embodiments 1 to 26, wherein the nucleobase sequence of the oligonucleotide is 100% complementary to an equal-length region of the CFTR transcript, as measured over the entire length of the oligonucleotide.
Embodiment 30. The compound of any of embodiments 1-29, wherein the nucleobase sequence of the antisense oligonucleotide comprises any one of SEQ ID NOs: 1 to 144, SEQ ID NO: 150, or SEQ ID NOs: 174-181.
Embodiment 31. The compound of any of embodiments 1-30, wherein the modified oligonucleotide comprises at least one modified nucleoside.
Embodiment 32. The compound of embodiment 31, wherein at least one modified nucleoside comprises a modified sugar moiety.
Embodiment 33. The compound of embodiment 32, wherein at least one modified sugar moiety is a 2 ‘-substituted sugar moiety.
Embodiment 34. The compound of embodiment 33, wherein the 2’-substitutent of at least one 2 ‘-substituted sugar moiety is selected from among: 2’-OMe, 2′-F, and 2′-MOE.
Embodiment 35. The compound of any of embodiments 31-34, wherein the 2′-substiuent of at least one 2 ‘-substituted sugar moiety is a 2’-MOE.
Embodiment 36. The compound of any of embodiments 1-47, wherein at least one modified sugar moiety is a bicyclic sugar moiety.
Embodiment 37. The compound of embodiment 36, wherein at least one bicyclic sugar moiety is LNA or cEt.
Embodiment 38. The compound of any of embodiments 1-37, wherein at least one sugar moiety is a sugar surrogate.
Embodiment 39. The compound of embodiment 38, wherein at least one sugar surrogate is a morpholino.
Embodiment 40. The compound of embodiment 38, wherein at least one sugar surrogate is a modified morpholino.
Embodiment 41. The compound of any of embodiments 1-40, wherein the modified oligonucleotide comprises at least 5 modified nucleosides, each independently comprising a modified sugar moiety.
Embodiment 42. The compound of embodiment 41, wherein the modified oligonucleotide comprises at least 10 modified nucleosides, each independently comprising a modified sugar moiety.
Embodiment 43. The compound of embodiment 41, wherein the modified oligonucleotide comprises at least 15 modified nucleosides, each independently comprising a modified sugar moiety.
Embodiment 44. The compound of embodiment 41, wherein each nucleoside of the modified oligonucleotide is a modified nucleoside, each independently comprising a modified sugar moiety
Embodiment 45. The compound of any of embodiments 1-44, wherein the modified oligonucleotide comprises at least two modified nucleosides comprising modified sugar moieties that are the same as one another.
Embodiment 46. The compound of any of embodiments 1-44, wherein the modified oligonucleotide comprises at least two modified nucleosides comprising modified sugar moieties that are different from one another.
Embodiment 47. The compound of any of embodiments 1-46, wherein the modified oligonucleotide comprises a modified region of at least 5 contiguous modified nucleosides.
Embodiment 48. The compound of any of embodiments 1 to 47, wherein the modified oligonucleotide comprises a modified region of at least 10 contiguous modified nucleosides.
Embodiment 49. The compound of any of embodiments 1 to 48, wherein the modified oligonucleotide comprises a modified region of at least 15 contiguous modified nucleosides.
Embodiment 50. The compound of any of embodiments 1 to 48, wherein the modified oligonucleotide comprises a modified region of at least 20 contiguous modified nucleosides.
Embodiment 51. The compound of any of embodiments 45 to 50, wherein each modified nucleoside of the modified region has a modified sugar moiety independently selected from among: 2′-F, 2′-OMe, 2′-MOE, cEt, LNA, morpholino, and modified morpholino.
Embodiment 52. The compound of any of embodiments 45 to 51 wherein the modified nucleosides of the modified region each comprise the same modification as one another.
Embodiment 53. The compound of embodiment 52, wherein the modified nucleosides of the modified region each comprise the same 2 ‘-substituted sugar moiety.
Embodiment 54. The compound of embodiment 52, wherein the 2’-substituted sugar moiety of the modified nucleosides of the region of modified nucleosides is selected from 2′-F. 2′-OMe, and 2′-MOE.
Embodiment 55. The compound of embodiment 54, wherein the 2 ‘-substituted sugar moiety of the modified nucleosides of the region of modified nucleosides is 2’-MOE.
Embodiment 56. The compound of embodiment 52, wherein the modified nucleosides of the region of modified nucleosides each comprise the same bicyclic sugar moiety.
Embodiment 57. The compound of embodiment 56, wherein the bicyclic sugar moiety of the modified nucleosides of the region of modified nucleosides is selected from LNA and cEt.
Embodiment 58. The compound of embodiment 50, wherein the modified nucleosides of the region of modified nucleosides each comprises a sugar surrogate.
Embodiment 59. The compound of embodiment 58, wherein the sugar surrogate of the modified nucleosides of the region of modified nucleosides is a morpholino.
Embodiment 60. The compound of embodiment 59, wherein the sugar surrogate of the modified nucleosides of the region of modified nucleosides is a modified morpholino.
Embodiment 61. The compound of any of embodiments 1 to 60, wherein the modified nucleotide comprises no more than 4 contiguous naturally occurring nucleosides.
Embodiment 62. The compound of any of embodiments 1 to 61, wherein each nucleoside of the modified oligonucleotide is a modified nucleoside.
Embodiment 63. The compound of embodiment 62, wherein each modified nucleoside comprises a modified sugar moiety.
Embodiment 64. The compound of embodiment 63, wherein the modified nucleosides of the modified oligonucleotide comprise the same modification as one another.
Embodiment 65. The compound of embodiment 64, wherein the modified nucleosides of the modified oligonucleotide each comprise the same 2 ‘-substituted sugar moiety.
Embodiment 66. The compound of embodiment 65, wherein the 2’-substituted sugar moiety of the modified oligonucleotide is selected from 2′-F, 2′-OMe, and 2′-MOE.
Embodiment 67. The compound of embodiment 65, wherein the 2 ‘-substituted sugar moiety of the modified oligonucleotide is 2’-MOE.
Embodiment 68. The compound of embodiment 64, wherein the modified nucleosides of the modified oligonucleotide each comprise the same bicyclic sugar moiety.
Embodiment 69. The compound of embodiment 68, wherein the bicyclic sugar moiety of the modified oligonucleotide is selected from LNA and cEt.
Embodiment 70. The compound of embodiment 64, wherein the modified nucleosides of the modified oligonucleotide each comprises a sugar surrogate.
Embodiment 71. The compound of embodiment 70, wherein the sugar surrogate of the modified oligonucleotide is a morpholino.
Embodiment 72. The compound of embodiment 70, wherein the sugar surrogate of the modified oligonucleotide is a modified morpholino.
Embodiment 73. The compound of any of embodiments 1 to 72, wherein the modified oligonucleotide comprises at least one modified internucleoside linkage.
Embodiment 74. The compound of embodiment 73, wherein each internucleoside linkage is a modified internucleoside linkage.
Embodiment 75. The compound of embodiment 73 or 74, comprising at least one phosphorothioate internucleoside linkage.
Embodiment 76. The compound of embodiment 73, wherein each internucleoside linkage is a modified internucleoside linkage and wherein each internucleoside linkage comprises the same modification.
Embodiment 77. The compound of embodiment 76, wherein each internucleoside linkage is a phosphorothioate internucleoside linkage.
Embodiment 78. The compound of any of embodiments 1 to 77, comprising at least one conjugate.
Embodiment 79. The compound of any of embodiments 1 to 78, consisting of the modified oligonucleotide.
Embodiment 80. The compound of any of embodiments 1 to 79, wherein the compound modulates splicing and/or expression of the CFTR transcript.
Embodiment 81. The compound of any of embodiments 1 to 80, having a nucleobase sequence comprising any of the sequences as set forth in SEQ ID Nos: 1 to 144, SEQ ID NO:150, or SEQ ID Nos: 174-181.
Embodiment 82. The compound of any of embodiments 1 to 81, having a nucleobase sequence comprising any of the sequences as set forth in SEQ ID Nos: 64, 65, 66, 71, 76, 78, 79, 81, 82, 84, 91, 92, 93, 94, 102, 111, 116, 117, 120, 122, 127, 128 or 129.
Embodiment 83. The compound of any of embodiments 1 to 81, having a nucleobase sequence comprising any of the sequences as set forth in SEQ ID Nos: 1, 4, 8, 9, 10, 12, 13, 17, 18, 19, 20, 22, 23, 24, 26, 27, 36, 37, 38, 42, 43, 44, 47, 48, 49, 50, 53, 55, 57, 59 or 60.
Embodiment 84a. The compound of any of embodiments 1 to 81, having a nucleobase sequence comprising SEQ ID NO: 174-177.
Embodiment 84b. The compound of any of embodiments 1 to 81, having a nucleobase sequence comprising SEQ ID NO: 178-181.
Embodiment 84c. The compound of any of embodiments 1-84b, further comprising one or more CFTR modulators.
Embodiment 84d. The compound of embodiment 84c, wherein the one or more CFTR modulators are selected from ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof.
Embodiment 85. A pharmaceutical composition comprising a compound according to any of embodiments 1-84d and a pharmaceutically acceptable carrier or diluent.
Embodiment 86. The pharmaceutical composition of embodiment 85, wherein the pharmaceutically acceptable carrier or diluent is sterile saline.
Embodiment 87. A method of modulating splicing and/or expression of a CFTR transcript in a cell comprising contacting the cell with a compound according to any of embodiments 1-86.

Embodiment 88. The method of embodiment 87, wherein the cell is in vitro.
Embodiment 89. The method of embodiment 87, wherein the cell is in an animal.
Embodiment 90. The method of any of embodiments 87 to 89, wherein the amount of CFTR mRNA without exon 4, exon 11, and/or exon 16 is increased.
Embodiment 91. The method of any of embodiments 87 to 89, wherein the amount of CFTR mRNA without exon 23 and/or exon 24 is increased.
Embodiment 92. The method of any of embodiments 87 to 89, wherein the amount of CFTR mRNA without exon 25 and/or exon 26 is increased.
Embodiment 93. The method of any of embodiments 87 to 92, wherein the CFTR transcript is transcribed from a CFTR gene.
Embodiment 94. A method of modulating the expression of CFTR in a cell, comprising contacting the cell with a compound according to any of embodiments 1-86.
Embodiment 95. The method of embodiment 94, wherein the cell is in vitro.
Embodiment 96. The method of embodiment 94, wherein the cell is in an animal.
Embodiment 97. A method comprising administering the compound according to any of embodiments 1-84d or the pharmaceutical composition of embodiments 85 or 86 to an animal.
Embodiment 98. The method of embodiment 97, wherein the administering step comprises delivering to the animal by inhalation, parenteral injection or infusion, intravenous injection, intrauterine, oral, subcutaneous or intramuscular injection, buccal, transdermal, transmucosal, and topical.
Embodiment 99. The method of embodiment 98, wherein the administration is by inhalation.
Embodiment 100. The method of any of embodiments 97-99, wherein the animal has one or more symptoms associated with cystic fibrosis.
Embodiment 101. The method of any of embodiments 97-99, wherein the administration results in amelioration of at least one symptom of cystic fibrosis.
Embodiment 102. The method of any of embodiments 97-101, wherein the animal is a mouse.
Embodiment 103. The method of any of embodiments 97-101, wherein the animal is a human.
Embodiment 104. A method of treating cystic fibrosis, comprising administering the compound according to any of embodiments 1-84d or the pharmaceutical composition of embodiments 85 or 86 to an animal in need thereof.
Embodiment 105. Use of the compound according to any of embodiments 1-84d or the pharmaceutical composition of embodiments 85 or 86 for the preparation of a medicament for use in the treatment of cystic fibrosis.
Embodiment 106. Use of the compound according to any of embodiments 1-84d or the pharmaceutical composition of embodiments 85 or 86 for the preparation of a medicament for use in the amelioration of one or more symptoms associated with cystic fibrosis.
Embodiment 107a. A compound comprising a modified oligonucleotide consisting of 8 to 30 linked nucleosides and having a nucleobase sequence comprising a complementary region, wherein the complementary region comprises at least 8 contiguous nucleobases complementary to an equal-length portion of a target region of a CFTR transcript.
Embodiment 107b. A compound comprising two or more modified oligonucleotides consisting of 8 to 30 linked nucleosides and each of the two more modified oligonucleotides having a nucleobase sequence comprising a complementary region, wherein the complementary region comprises at least 8 contiguous nucleobases complementary to an equal-length portion of a target region of a CFTR transcript.
Embodiment 108. The compound of embodiment 107, wherein the CFTR transcript comprises the nucleobase sequence of SEQ ID No. 130.
Embodiment 109. The compound of embodiment 107 or 108, wherein the complementary region of the modified oligonucleotide is 100% complementary to the target region.
Embodiment 110. The compound of any of embodiments 107-109, wherein the complementary region of the modified oligonucleotide(s) comprises at least 10 contiguous nucleobases.
Embodiment 111. The compound of any of embodiments 107-109, wherein the complementary region of the modified oligonucleotide(s) comprises at least 12 contiguous nucleobases.
Embodiment 112. The compound of any of embodiments 107-109, wherein the complementary region of the modified oligonucleotide(s) comprises at least 14 contiguous nucleobases.
Embodiment 113. The compound of any of embodiments 107-109, wherein the complementary region of the modified oligonucleotide(s) comprises at least 15 contiguous nucleobases.
Embodiment 114. The compound of any of embodiments 107-109, wherein the complementary region of the modified oligonucleotide(s) comprises at least 16 contiguous nucleobases.
Embodiment 115. The compound of any of embodiments 107-109, wherein the complementary region of the modified oligonucleotide(s) comprises at least 17 contiguous nucleobases.
Embodiment 116. The compound of any of embodiments 107-109, wherein the complementary region of the modified oligonucleotide(s) comprises at least 18 contiguous nucleobases.
Embodiment 117. The compound of any of embodiments 107-116, wherein the nucleobase sequence of the modified oligonucleotide(s) is at least 80% complementary to an equal-length region of the CFTR transcript, as measured over the entire length of the oligonucleotide.
Embodiment 118. The compound of any of embodiments 107-116, wherein the nucleobase sequence of the modified oligonucleotide(s) is at least 90% complementary to an equal-length region of the CFTR transcript, as measured over the entire length of the oligonucleotide.
Embodiment 119. The compound of any of embodiments 107-116, wherein the nucleobase sequence of the modified oligonucleotide(s) is 100% complementary to an equal-length region of the CFTR transcript, as measured over the entire length of the oligonucleotide.
Embodiment 120. The compound of any of embodiments 107-119, wherein the target region is within intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24, or intron 24 of human CFTR, or wherein the target region is about 25 nucleobases upstream or about 25 nucleobases downstream of intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24, intron 24, exon 25, intron 25, exon 26, or intron 26 of human CFTR.
Embodiment 121. The compound of embodiment 120, wherein the target region is within exon 4 or exon 11 of human CFTR, or is about 25 nucleobases upstream or about 25 nucleobases downstream of exon 4 or exon 11 of human CFTR.
Embodiment 122a. The compound of embodiment 120, wherein the target region is within exon 23 or exon 24 of human CFTR, or is about 25 nucleobases upstream or about 25 nucleobases downstream of exon 23 or exon 24 of human CFTR.
Embodiment 122b. The compound of embodiment 120, wherein the target region is within exon 25 or exon 26 of human CFTR, or is about 25 nucleobases upstream or about 25 nucleobases downstream of exon 25 or exon 26 of human CFTR.
Embodiment 123. The compound of any of embodiments 107-119, wherein the target region is within intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24 or intron 24 of mouse CFTR, or is about 25 nucleobases upstream or about 25 nucleobases downstream of intron 1, exon 2, intron 2, intron 3, exon 4, intron 4, exon 5, intron 6, exon 7, intron 7, exon 9, intron 9, exon 10, intron 10, exon 11, intron 11, intron 12, exon 13, intron 13, intron 14, exon 15, intron 15, exon 16, intron 16, intron 19, exon 20, intron 20, intron 21, exon 22, intron 22, exon 23, intron 23, exon 24 or intron 24 of mouse CFTR.
Embodiment 124. The compound of any of embodiments 107-119, wherein the modified oligonucleotide has a nucleobase sequence comprising any of the sequences as set forth in SEQ ID NOs: 1-144, SEQ ID NO: 150, and SEQ ID NO: 174-181.
Embodiment 125. The compound of any of embodiments 107-119, wherein the modified oligonucleotide has a nucleobase sequence consisting of the nucleobase sequence of any one of SEQ ID NOs: 1-144, SEQ ID NO:150, and SEQ ID NO: 174-181.
Embodiment 126. The compound of any of embodiments 107-119, wherein the modified oligonucleotide has a nucleobase sequence comprising the nucleobase sequence of SEQ ID NO: 64, 65, 66, 71, 76, 78, 79, 81, 82, 84, 91, 92, 93, 94, 97, 99, 100, 102, 103, 104, 111, 114, 116, 117, 120, 122, 127, 128, 129, 150, 174, 175, 176, 177, 178, 179, 180, or 181.
Embodiment 127. The compound of any of embodiments 107-119, wherein the modified oligonucleotide has a nucleobase sequence consisting of the nucleobase sequence of SEQ ID NO: 64, 65, 66, 71, 76, 78, 79, 81, 82, 84, 91, 92, 93, 94, 97, 99, 100, 102, 103, 104, 111, 114, 116, 117, 120, 122, 127, 128, 129, 150, 174, 175, 176, 177, 178, 179, 180, or 181.
Embodiment 128a. The compound of any of embodiments 107-119, wherein the modified oligonucleotide has a nucleobase sequence comprising the nucleobase sequence of SEQ ID NO: 91, 97, 99, 100, 103, 104, 110, 114, 126, 127, 128, 129, 150, 174, 175, 176, 177, 178, 179, 180, or 181.
Embodiment 128b. The compound of any of embodiments 107-119, wherein the modified oligonucleotide has a nucleobase sequence comprising the nucleobase sequence of SEQ ID NO: 65-70, 123-126, or 127-129, 174, 175, 176, 177, 178, 179, 180, or 181.
Embodiment 129a. The compound of embodiment 107-119, wherein the modified oligonucleotide has a nucleobase sequence consisting of the nucleobase sequence of SEQ ID NO: 91, 97, 99, 100, 103, 104, 110, 114, 126, 127, 128, 129, 150, 174, 175, 176, 177, 178, 179, 180, or 181.
Embodiment 129b. The compound of embodiment 107-119, wherein the modified oligonucleotide has a nucleobase sequence consisting of the nucleobase sequence of SEQ ID NO: 65-70, 123-126, 127-129, 174, 175, 176, 177, 178, 179, 180, or 181.
Embodiment 130. The compound of any of embodiments 107-129, wherein the modified oligonucleotide comprises at least one modified nucleoside.
Embodiment 131. The compound of any of embodiments 107-130, wherein each nucleoside of the modified oligonucleotide is a modified nucleoside selected from among: 2′-OMe, 2′-F, and 2′-MOE or a sugar surrogate.
Embodiment 132. The compound of embodiment 132, wherein the modified nucleoside is 2′-MOE.
Embodiment 133. The compound of embodiment 132, wherein the modified nucleoside is a morpholino.
Embodiment 134. The compound of embodiment 131, wherein at least one modified nucleoside comprises a modified sugar moiety.
Embodiment 135. The compound of embodiment 134, wherein at least one modified sugar moiety is a 2 ‘-substituted sugar moiety.
Embodiment 136. The compound of embodiment 135, wherein the 2’-substitutent of at least one 2 ‘-substituted sugar moiety is selected from among: 2’-OMe, 2′-F, and 2′-MOE.
Embodiment 137. The compound of any of embodiments 135-136, wherein the 2′-substiuent of at least one 2 ‘-substituted sugar moiety is a 2’-MOE.
Embodiment 138. The compound of any of embodiments 107-137, wherein at least one modified sugar moiety is a bicyclic sugar moiety.
Embodiment 139. The compound of embodiment 138, wherein at least one bicyclic sugar moiety is LNA or cEt.
Embodiment 140. The compound of any of embodiments 107-139, wherein at least one sugar moiety is a sugar surrogate.
Embodiment 141. The compound of embodiment 140, wherein at least one sugar surrogate is a morpholino.
Embodiment 142. The compound of embodiment 141, wherein at least one sugar surrogate is a modified morpholino.
Embodiment 143. The compound of any of embodiments 107-142, wherein the modified oligonucleotide comprises at least 5 modified nucleosides, each independently comprising a modified sugar moiety.
Embodiment 144. The compound of any of embodiments 107-143, wherein the modified oligonucleotide comprises at least 10 modified nucleosides, each independently comprising a modified sugar moiety.
Embodiment 145. The compound of any of embodiments 107-143, wherein the modified oligonucleotide comprises at least 15 modified nucleosides, each independently comprising a modified sugar moiety.
Embodiment 146. The compound of any of embodiments 107-143, wherein each nucleoside of the modified oligonucleotide is a modified nucleoside, each independently comprising a modified sugar moiety.
Embodiment 147. The compound of any of embodiments 107-146, wherein the modified oligonucleotide comprises at least two modified nucleosides comprising modified sugar moieties that are the same as one another.
Embodiment 148. The compound of any of embodiments 107-146, wherein the modified oligonucleotide comprises at least two modified nucleosides comprising modified sugar moieties that are different from one another.
Embodiment 149. The compound of any of embodiments 107-148, wherein the modified oligonucleotide comprises a modified region of at least 5 contiguous modified nucleosides.
Embodiment 150. The compound of any of embodiments 107-148, wherein the modified oligonucleotide comprises a modified region of at least 10 contiguous modified nucleosides.
Embodiment 151. The compound of any of embodiments 107-148, wherein the modified oligonucleotide comprises a modified region of at least 15 contiguous modified nucleosides.
Embodiment 152. The compound of any of embodiments 107-148, wherein the modified oligonucleotide comprises a modified region of at least 16 contiguous modified nucleosides.
Embodiment 153. The compound of any of embodiments 107-148, wherein the modified oligonucleotide comprises a modified region of at least 17 contiguous modified nucleosides.
Embodiment 154. The compound of any of embodiments 107-148, wherein the modified oligonucleotide comprises a modified region of at least 18 contiguous modified nucleosides.
Embodiment 155. The compound of any of embodiments 107-148, wherein the modified oligonucleotide comprises a modified region of at least 20 contiguous modified nucleosides.
Embodiment 156. The compound of any of embodiments 149-155, wherein each modified nucleoside of the modified region has a modified sugar moiety independently selected from among: 2′-F, 2′-OMe, 2′-MOE, cEt, LNA, morpholino, and modified morpholino.
Embodiment 157. The compound of any of embodiments 149-156, wherein the modified nucleosides of the modified region each comprise the same modification as one another.
Embodiment 158. The compound of embodiment 157, wherein the modified nucleosides of the modified region each comprise the same 2 ‘-substituted sugar moiety.
Embodiment 159. The compound of embodiment 157, wherein the 2’-substituted sugar moiety of the modified nucleosides of the region of modified nucleosides is selected from 2′-F. 2′-OMe, and 2′-MOE.
Embodiment 160. The compound of embodiment 157, wherein the 2 ‘-substituted sugar moiety of the modified nucleosides of the region of modified nucleosides is 2’-MOE.
Embodiment 161. The compound of embodiment 157, wherein the modified nucleosides of the region of modified nucleosides each comprise the same bicyclic sugar moiety.
Embodiment 162. The compound of embodiment 161, wherein the bicyclic sugar moiety of the modified nucleosides of the region of modified nucleosides is selected from LNA and cEt.
Embodiment 163. The compound of embodiment 157, wherein the modified nucleosides of the region of modified nucleosides each comprises a sugar surrogate.
Embodiment 164. The compound of embodiment 163, wherein the sugar surrogate of the modified nucleosides of the region of modified nucleosides is a morpholino.
Embodiment 165. The compound of embodiment 163, wherein the sugar surrogate of the modified nucleosides of the region of modified nucleosides is a modified morpholino.
Embodiment 166. The compound of any of embodiments 107-165, wherein the modified nucleotide comprises no more than 4 contiguous naturally occurring nucleosides.
Embodiment 167. The compound of any of embodiments 107-165, wherein each nucleoside of the modified oligonucleotide is a modified nucleoside.
Embodiment 168. The compound of embodiment 167, wherein each modified nucleoside comprises a modified sugar moiety.
Embodiment 169. The compound of embodiment 168, wherein the modified nucleosides of the modified oligonucleotide comprise the same modification as one another.
Embodiment 170. The compound of embodiment 169, wherein the modified nucleosides of the modified oligonucleotide each comprise the same 2 ‘-substituted sugar moiety.
Embodiment 171. The compound of embodiment 170, wherein the 2’-substituted sugar moiety of the modified oligonucleotide is selected from 2′-F. 2′-OMe, and 2′-MOE.
Embodiment 172. The compound of embodiment 170, wherein the 2 ‘-substituted sugar moiety of the modified oligonucleotide is 2’-MOE.
Embodiment 173. The compound of embodiment 171, wherein the modified nucleosides of the modified oligonucleotide each comprise the same bicyclic sugar moiety.
Embodiment 174. The compound of embodiment 173, wherein the bicyclic sugar moiety of the modified oligonucleotide is selected from LNA and cEt.
Embodiment 175. The compound of embodiment 169, wherein the modified nucleosides of the modified oligonucleotide each comprises a sugar surrogate.
Embodiment 176. The compound of embodiment 175, wherein the sugar surrogate of the modified oligonucleotide is a morpholino.
Embodiment 177. The compound of embodiment 175, wherein the sugar surrogate of the modified oligonucleotide is a modified morpholino.
Embodiment 178. The compound of any of embodiments 107-177, wherein the modified oligonucleotide comprises at least one modified internucleoside linkage.
Embodiment 179. The compound of embodiment 178, wherein each internucleoside linkage is a modified internucleoside linkage.
Embodiment 180. The compound of embodiment 178 or 179, comprising at least one phosphorothioate internucleoside linkage.
Embodiment 181. The compound of embodiment 179, wherein each internucleoside linkage is a modified internucleoside linkage and wherein each internucleoside linkage comprises the same modification.
Embodiment 182. The compound of embodiment 181, wherein each internucleoside linkage is a phosphorothioate internucleoside linkage.
Embodiment 183. The compound of any of embodiments 107-182, comprising at least one conjugate.
Embodiment 184. The compound of any of embodiments 107-183, consisting of the modified oligonucleotide.
Embodiment 185. The compound of any of embodiments 107-184, wherein the compound modulates splicing and/or expression of the CFTR transcript.
Embodiment 186. A pharmaceutical composition comprising a compound according to any of embodiments 107-186 and a pharmaceutically acceptable carrier or diluent.
Embodiment 187. The pharmaceutical composition of embodiment 186, wherein the pharmaceutically acceptable carrier or diluent is sterile saline.
Embodiment 188. A method of modulating splicing of a CFTR transcript in a cell comprising contacting the cell with a compound according to any of embodiments 107-187.
Embodiment 189. The method of embodiment 188, wherein the cell is in vitro.
Embodiment 190. The method of embodiment 188, wherein the cell is in an animal.
Embodiment 191. The method of any of embodiments 188-190, wherein the amount of CFTR mRNA without exon 4 and/or exon 16 is increased.
Embodiment 192. The method of any of embodiments 188-190, wherein the amount of CFTR mRNA without exon 23 and/or exon 24 is increased.
Embodiment 193. The method of any of embodiments 188-190, wherein the amount of CFTR mRNA without exon 25 and/or exon 26 is increased.
Embodiment 194. The method of any of embodiments 188-193, wherein the CFTR transcript is transcribed from a CFTR gene.
Embodiment 195. A method of modulating the expression of CFTR in a cell, comprising contacting the cell with a compound according to any of embodiments 107-185.
Embodiment 196. The method of embodiment 195, wherein the cell is in vitro.
Embodiment 197. The method of embodiment 195, wherein the cell is in an animal.
Embodiment 198. A method comprising administering the compound of any of embodiments 107-185 to an animal.
Embodiment 199. The method of embodiment 198, wherein the administering step comprises delivering to the animal by inhalation, parenteral injection or infusion, oral, subcutaneous or intramuscular injection, buccal, transdermal, transmucosal and topical.
Embodiment 200. The method of embodiment 198, wherein the administration is inhalation.
Embodiment 201. The method of any of embodiments 198-200, wherein the animal has one or more symptoms associated with cystic fibrosis.
Embodiment 202. The method of any of embodiments 198-200, wherein the administration results in amelioration of at least one symptom of cystic fibrosis.
Embodiment 203. The method of any of embodiments 198-202, wherein the animal is a mouse.
Embodiment 204. The method of any of embodiments 198-202, wherein the animal is a human.
Embodiment 205. A method of preventing or slowing one or more symptoms associated with cystic fibrosis, comprising administering the compound according to any of embodiments 107-185 to an animal in need thereof.
Embodiment 205a. The method of embodiment 205, further comprising administering one or more Cystic fibrosis transmembrane conductance regulator (CFTR) modulators.
Embodiment 205b. The method of embodiment 205a, wherein the one or more CFTR modulators are selected from ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof.
Embodiment 206. The method of embodiment 205, wherein the animal is a human.
Embodiment 207. Use of the compound according to any of embodiments 107-185 for the preparation of a medicament for use in the treatment of cystic fibrosis.
Embodiment 208. Use of the compound according to any of embodiments 107-185 for the preparation of a medicament for use in the amelioration of one or more symptoms associated with cystic fibrosis.

These and other features and advantages of the present disclosure will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

A better understanding of the disclosure may be obtained in light of the following drawings which are set forth for illustrative purposes, and should not be construed as limiting the scope of the disclosure in any way.

FIG. 1A shows a map of the murine/mouse CFTR gene. Boxes represent exons and lines represent introns. The exons that can be skipped or spliced out of the mature mRNA and still maintain the open reading frame of the mRNA are shaded. The CFTR mRNAs lacking any one of these exons will code for a full-length CFTR protein with an internal deletion of the specific targeted exon sequence.

FIG. 1B shows antisense oligonucleotides induce skipping of targeted exons 2, 4, 5, 7 and 9 of the murine CFTR gene-derived pre-mRNA. Polyacrylamide gel images of radio-labeled reverse-transcription/polymerase chain reaction (RT-PCR) products separated by electrophoresis are shown. RT-PCR was performed on RNA isolated from a mouse primary cell line treated with the indicated ASO or treated with vehicle (saline) only (−). The products were amplified with primers specific to exons flanking the specific ASO-targeted exon in order to resolve the full-length (FL) and exon-skipped (*) products. The targeted exon is indicated at the top of the gel image. PCR products were quantitated and the percent of the products that are skipped [Exon skipped/(Full-length+skipped)]x 100 is shown below the gel image.

FIG. 1C shows antisense oligonucleotides induce skipping of targeted exons 10, 11, 13 and 15 of the murine CFTR gene-derived pre-mRNA. Polyacrylamide gel images of radio-labeled reverse-transcription/polymerase chain reaction (RT-PCR) products separated by electrophoresis are shown. RT-PCR was performed on RNA isolated from a mouse primary cell line treated with the indicated ASO or treated with vehicle (saline) only (−). The products were amplified with primers specific to exons flanking the specific ASO-targeted exon in order to resolve the full-length (FL) and exon-skipped (*) products. The targeted exon is indicated at the top of the gel image. PCR products were quantitated and the percent of the products that are skipped [Exon skipped/(Full-length+skipped)]×100 is shown below the gel image.

FIG. 1D shows antisense oligonucleotides induce skipping of targeted exons 20, 22, 23 and 24 of the murine CFTR gene-derived pre-mRNA. Polyacrylamide gel images of radio-labeled reverse-transcription/polymerase chain reaction (RT-PCR) products separated by electrophoresis are shown. RT-PCR was performed on RNA isolated from a mouse primary cell line treated with the indicated ASO or treated with vehicle (saline) only (−). The products were amplified with primers specific to exons flanking the specific ASO-targeted exon in order to resolve the full-length (FL) and exon-skipped (*) products. The targeted exon is indicated at the top of the gel image. PCR products were quantitated and the percent of the products that are skipped [Exon skipped/(Full-length+skipped)]×100 is shown below the gel image.

FIG. 2A shows a map of the human CFTR gene. Boxes represent exons and lines represent introns. The exons that can be skipped or spliced out of the mature mRNA and still maintain the open reading frame of the mRNA are shaded. The CFTR mRNAs lacking any one of these exons will code for a full-length CFTR protein with an internal deletion of the specific targeted exon sequence.

FIG. 2B show antisense oligonucleotides induce skipping of targeted exons 2, 4, 5 and 7 of the human CFTR gene-derived pre-mRNA. Agarose gel images of reverse-transcription/polymerase chain reaction (RT-PCR) products separated by electrophoresis are shown. RT-PCR was performed on RNA isolated from human T84 epithelial cells treated with the indicated ASO or treated with vehicle (saline) only (−) or a reaction lacking cDNA (−RT). The products were amplified with primers specific to exons flanking the specific ASO-targeted exon in order to resolve the full-length (FL; +) and exon-skipped (*) products. The targeted exon is indicated at the bottom of the gel and by the first numbers in the name of the ASOs.

FIG. 2C show antisense oligonucleotides induce skipping of targeted exons 9, 10, 11, 13 and 15 of the human CFTR gene-derived pre-mRNA. Agarose gel images of reverse-transcription/polymerase chain reaction (RT-PCR) products separated by electrophoresis are shown. RT-PCR was performed on RNA isolated from human T84 epithelial cells treated with the indicated ASO or treated with vehicle (saline) only (−) or a reaction lacking cDNA (−RT). The products were amplified with primers specific to exons flanking the specific ASO-targeted exon in order to resolve the full-length (FL; +) and exon-skipped (*) products. The targeted exon is indicated at the bottom of the gel and by the first numbers in the name of the ASOs.

FIG. 2D show antisense oligonucleotides induce skipping of targeted exons 20, 22, 23 and 24 of the human CFTR gene-derived pre-mRNA. Agarose gel images of reverse-transcription/polymerase chain reaction (RT-PCR) products separated by electrophoresis are shown. RT-PCR was performed on RNA isolated from human T84 epithelial cells treated with the indicated ASO or treated with vehicle (saline) only (−) or a reaction lacking cDNA (−RT). The products were amplified with primers specific to exons flanking the specific ASO-targeted exon in order to resolve the full-length (FL; +) and exon-skipped (*) products. The targeted exon is indicated at the bottom of the gel and by the first numbers in the name of the ASOs.

FIG. 3A shows a schematic of the splicing pattern of human CFTR c.2789+5G>A without and with ASO targeting. Boxes are exons and lines are introns. Diagonal lines indicate splicing pathway

FIG. 3B demonstrates that antisense oligonucleotides correct splicing of human CFTR exon 16 with c.2789+5G>A mutation. Polyacrylamide gel images of reverse-transcription/polymerase chain reaction (RT-PCR) products were separated by electrophoresis. RT-PCR was performed on RNA isolated from human lymphoblast cell line GM11859, whose donor is homozygous for G-to-A substitution at nucleotide 2789+5 in intron 16 which results in an mRNA splicing defect (2789+5G>A). Cells were treated with the indicated ASO. The products were amplified with primers specific to exons flanking the specific ASO-targeted exon in order to resolve the full-length (FL) and exon-skipped products. ASO 16-8 was effective at correcting exon 16 splicing of CFTRc.2789+5G>A.

FIG. 4 shows the genomic DNA of exon 2 in human CFTR and surrounding introns (the sequence of FIG. 4 is given the sequence identifier SEQ ID NO: 131).

FIG. 5 shows the genomic DNA of exon 4 in human CFTR and surrounding introns (the sequence of FIG. 5 is given the sequence identifier SEQ ID NO: 132).

FIG. 6 shows the genomic DNA of exon 5 in human CFTR and surrounding introns (the sequence of FIG. 6 is given the sequence identifier SEQ ID NO: 133).

FIG. 7 shows the genomic DNA of exon 7 in human CFTR and surrounding introns (the sequence of FIG. 7 is given the sequence identifier SEQ ID NO: 134).

FIG. 8 shows the genomic DNA of exon 9 in human CFTR and surrounding introns (the sequence of FIG. 8 is given the sequence identifier SEQ ID NO: 135).

FIG. 9 shows the genomic DNA of exon 10 in human CFTR and surrounding introns (the sequence of FIG. 9 is given the sequence identifier SEQ ID NO: 136).

FIG. 10 shows the genomic DNA of exon 11 in human CFTR and surrounding introns (the sequence of FIG. 10 is given the sequence identifier SEQ ID NO: 137).

FIG. 11 shows the genomic DNA of exon 13 in human CFTR and surrounding introns (the sequence of FIG. 11 is given the sequence identifier SEQ ID NO: 138).

FIG. 12 shows the genomic DNA of exon 15 in human CFTR and surrounding introns (the sequence of FIG. 12 is given the sequence identifier SEQ ID NO: 139).

FIG. 13 shows the genomic DNA of exon 16 in human CFTR and surrounding introns (the sequence of FIG. 13 is given the sequence identifier SEQ ID NO: 140).

FIG. 14 shows the genomic DNA of exon 20 in human CFTR and surrounding introns (the sequence of FIG. 14 is given the sequence identifier SEQ ID NO: 141).

FIG. 15 shows the genomic DNA of exon 22 in human CFTR and surrounding introns (the sequence of FIG. 15 is given the sequence identifier SEQ ID NO: 142).

FIG. 16 shows the genomic DNA of exon 23 in human CFTR and surrounding introns (the sequence of FIG. 16 is given the sequence identifier SEQ ID NO: 143).

FIG. 17 shows the genomic DNA of exon 24 in human CFTR and surrounding introns (the sequence of FIG. 17 is given the sequence identifier SEQ ID NO: 144).

FIG. 18A shows a comparison of the AUC forskolin-stimulated HCAI-CFTR exon deletion channel activity in Fischer Rat Thyroid (FRT) cells to HCAI empty vector. Error bars represent SEM (*p<. 05. *** p<. 001, n=4, two-tailed t-test compared to HCAI empty vector).

FIG. 18B shows representative Gt traces of CFTR exon 4, exon 7, exon 23, and exon 24 deletion constructs in Fischer Rat Thyroid (FRT) cells in comparison to HCAI empty vector.

FIG. 19A shows a radioactive RT-PCR of CFTR RNA isolated from hippocampus that demonstrates that ASO 5-1 induces CFTR exon 5 skipping in vivo. Splice isoforms are labeled and exon 5 skipping quantification is shown at the bottom.

FIG. 19B shows a quantitation of the RT-PCR analysis of the RNA exon 5 skipping induced by ASO treatment. Approximately 60% of mouse CFTR gene exon 5 is skipped when mice are treated with ASO 5-1 by ICV injection.

FIG. 20A shows a schematic for antisense oligonucleotides to correct CFTR 2789+5 G>A splicing mutation.

FIG. 20B shows a radioactive RT-PCR of CFTR RNA isolated from 2789+5 patient lymphoblast cells transfected with ASOs (15 μM) for 48 hours. The results demonstrate correction of CFTR splicing in 2789+5 patient lymphoblast cells using ASOs. The CFTR spliced isoforms are labelled. T84 cells were analyzed as a positive control for wild-type CFTR splicing.

FIG. 20C shows a quantitation of the RT-PCR analysis of the RNA splice correction induced by ASO treatment in patient lymphoblast cells.

FIG. 21A shows a schematic for antisense oligonucleotides to correct CFTR 3272-26A>G splicing mutation.

FIG. 21B shows a radioactive RT-PCR of CFTR RNA isolated from 3272-26A>G patient lymphoblast cells transfected with ASOs (15 μM) for 48 hours. The results demonstrate correction of CFTR splicing in 3272-26A>G patient lymphoblast cells using ASOs. The CFTR spliced isoforms are labelled. T84 cells were analyzed as a positive control for wild-type CFTR splicing.

FIG. 22A shows a diagram of ASOs used for the correction of CFTR splicing in 3849+10 kb patient lymphoblast cells using ASOs. The +10C>T mutation is labeled.

FIG. 22B shows the results of a RT-PCR assay of CFTR RNA isolated from 3849+10 kb patient lymphoblast cells transfected with ASOs (15 μM) for 48 hours. Results indicate a correction of CFTR splicing in 3849+10 kb patient lymphoblast cells using the ASOs. CFTR spliced isoforms are labeled. T84 cells were analyzed as a positive control for wild-type CFTR splicing (FL=Full-Length).

FIG. 22C shows a quantitation of the RT-PCR analysis of the RNA splice correction induced by ASO treatment in patient lymphoblast cells. The results indicate about a four-fold reduction of inclusion of the cryptic exon, resulting in approximately 93% of the CFTR transcripts being full-length.

FIG. 23A shows that ASO-+10 kb rescues CFTR function similar to Corr951 (VX-770) in patient HBE cells. The graph depicts the area under the curve (AUC) of time from forskolin+VX-770-stimulation of CFTR channels following indicated treatment. Error bars represent SEM (two-tailed t-test, n=2).

FIG. 23B shows ASO-+10 kb rescues CFTR function similar to Corr951 in patient HBE cells. Representative Ieq traces of treatment (Corr951 or ASO-+10 kb) compared to control (ASO-C, top, or DMSO, bottom).

FIG. 24A shows a diagram of primer sets used to analyze splice correction by ASO-10+kb. Primer set A-B is designed to amplify ASO corrected WT isoform splicing specific to the splice mutant allele. Primer set C-D is designed to analyze the amount of uncorrected mutant splicing

FIG. 24B show a quantification of total mRNA transcribed from the CFTR 3849+10 KB allele indicates an increase with ASO-+10 kb treatment (A-B primer set shown in FIG. 24A).

FIG. 24C shows a quantification of mutant, cryptically spliced mRNA isoform shows decrease of aberrant mRNA with ASO-+10 kb treatment (C-D primer set shown in FIG. 24A).

FIG. 25A shows a schematic of CFTR exon 23 region relative to its position within NBD2 and the position of the CFTR-W1282X mutation.

FIG. 25B shows a schematic of the CFTR protein encoded by the CFTR-W1282X and CFTR-Δ23 expression plasmids.

FIG. 25C shows average conductance traces from FRT cells stably transfected with the CFTR-Δ23 or CFTR-W1282X plasmids, or empty vector. Cells were pre-treated with vehicle (solid lines) or C18 (dashed lines) for 24 hours. Baseline conductance measurements were taken for 20 minutes. The time of compound additions is indicated.

FIG. 25D shows average AUC/min was quantified for the forskolin or forskolin+VX-770 20 minute test periods for each construct (+SEM; CFTR-W1282X, N=3, CFTR-Δ23, N=6, empty vector, N=5; two-way ANOVA: Sidak's multiple comparison test within groups: **** p<. 0001).

FIG. 25E shows immunoblot analysis of CFTR protein, Band C and Band B, isolated from in FRT cells stably transfected with CFTR-WT, CFTR-Δ23, and CFTR-W1282X constructs. β-actin was used as a control for protein expression.

FIG. 25F shows quantification of total CFTR C band from E, normalized to β-actin.

FIG. 25G shows average conductance traces from FRT cells stably transfected with CFTR-A23, CFTR-WT, or empty vector. Cells were pre-treated with vehicle (solid lines), C18 (dashed lines) or VX-661+VX-445 (dotted lines) for 24 hours. Baseline conductance measurements were taken for 20 minutes. The addition of compounds is indicated.

FIG. 25H shows AUC of CFTR-Δ23 conductance was calculated for each modulator treatment (indicated on the X-axis) as the percent CFTR-WT untreated with modulators (+SEM; N=3 except VX-661+VX-445 treatment N=1; one-way ANOVA: Sidak′s multiple comparison test: * p<. 05).

FIG. 26A shows a schematic of the CFTR disruption caused by the W1282X-CFTR mutation in exon 23. The G>A mutation creates a premature termination codon (PTC) in exon 23 leading to degradation of the mRNA transcript via nonsense mediated mRNA decay, drastically reducing production of semi-functional truncated W1282X-CFTR protein. An ASO designed to induced exon 23, the exon that encodes W1282X, eliminates the PTC, restores CFTR mRNA stability, and increases production of semi-functional CFTR protein.

FIG. 26B shows a diagram of ASO target sites on human CFTR exon 23 pre-mRNA. ASOs are shown above the complementary CFTR sequence. The location of the W1282X-CFTR mutation is indicated by the red lettering. The natural and cryptic 5′ splice sites are indicated.

FIG. 26C shows RT-PCR analysis of exon 23 splicing in W1282X-CFTR expressing immortalized human bronchial epithelial (hBE) cells treated with ASOs (80 μM each). β-actin was analyzed as control. The percent of total CFTR mRNA with exon 23 skipped is shown in Table 7.

FIG. 26D shows AUC of CFTR-Δ23 conductance was calculated for each treatment (indicated on the X-axis) as the percent ASO-C treated (+SEM; N=2; one-way ANOVA: Sidak's multiple comparison test; * p<. 05); see Table 8 for quantitation.

FIG. 27A shows that increasing the concentration of each ASO delivered to the cells resulted in increasing exon 23 skipping.

FIG. 27B shows that increasing the concentration of each ASO delivered to the cells resulted in increasing chloride conductance.

FIG. 27C shows that the combination of ASO 23-3 and 23-4 eliminate cryptic splicing and induce W1282X-CFTR exon 23 skipping and rescue activity in a dose dependent manner in 16HBEge-W1282X-CFTR cells.

FIG. 28A shows a schematic of CFTR exon 23 in relation to its position within NBD2 and the position of the CFTR-W1282X mutation. Symmetric exons are in grey.

FIG. 28B shows a schematic of the CFTR-W1282X and CFTR-Δ23 constructs transfected into FRT cells.

FIG. 28C shows CFTR-Δ23 function is comparable to CFTR-W1282X and is responsive to CF modulators. Average conductance traces from FRT cells stably transfected with CFTR-Δ23, CFTR-W1282X, or empty vector. Cells were pre-treated with vehicle (DMSO, solid lines), C18 (dotted lines), or VX-445+VX-661 (dashed lines). The time of compound additions (Forskolin, VX-770, or Inh-172) is indicated.

FIG. 28D shows CFTR-Δ23 function is comparable to CFTR-W1282X and is responsive to CF modulators. Average area under the curve (AUC) was quantified for the forskolin and VX-770 test periods for each construct. Error bars are ±SEM. Two-way ANOVA: Dunnett's multiple comparison test to vehicle within groups. **** p<0.0001. CFTR-Δ23: DMSO and VX-770, N=11; C18, N=6; VX-445+VX-661, N=5. CFTR-W1282X: DMSO and VX-770, N=9; C18, N=5; VX-445+VX-661, N=4, empty vector: DMSO and VX-770, N=8; C18, N=5; VX-445+VX-661, N=3.

FIG. 28E shows CFTR-Δ23 function is comparable to CFTR-W1282X and is responsive to CF modulators. Immunoblot analysis of CFTR protein, Bands C and B, isolated from in FRT cells stably transfected with empty vector, CFTR-WT, CFTR-W1282X, or CFTR-Δ23 constructs treated with vehicle or C18. β-actin was used as a control for protein expression.

FIG. 28F shows CFTR-Δ23 function is comparable to CFTR-W1282X and is responsive to CF modulators. Quantification of total CFTR C band from (FIG. 28E), normalized to β-actin. Error bars are ±SEM. Empty vector: N=1, CFTR-WT: N=4, CFTR-W1282X: N=2, CFTR-Δ23: DMSO. N=4: C18N=3.

FIG. 29A shows a schematic of the CFTR dysfunction caused by the W1282X mutation in exon 23. The c.3846 G>A (W1282X) mutation creates a premature termination codon (PTC) in exon 23 leading to degradation of the transcript via NMD and reducing translation of a semi-functional truncated CFTR protein (indicated by down arrows). ASO induced exon 23 skipping, eliminates the PTC, restores CFTR mRNA stability and increases CFTR expression (indicated by up arrows).

FIG. 29B shows a diagram of ASO target sites (green lines) on human CFTR exon 23 pre-mRNA. The location of the CFTR-W1282X mutation is indicated by a red vertical line.

FIG. 29C shows ASOs induce CFTR-W1282X exon 23 skipping (C) RT-PCR analysis of CFTR exon 23 splicing in a CFF16HBEge-W1282X cell line treated with the indicated ASO (10 μM). Exon 23 skipping or cryptic splice site activation was quantified [423 or cryptic/(A23+cryptic+W1282X) x100] and is indicated below each lane.

FIG. 29D shows a sequence alignment of ASO-23A (SEQ ID NO: 126) and ASO-23B (SEQ ID NO: 125) to exon 23.

FIG. 29E shows ASOs induce CFTR-W1282X exon 23 skipping. RT-PCR analysis of exon 23 splicing in CFF16HBEge-W1282X cells treated with ASO-23A, ASO-23B, ASO-23A+ASO-23B (ASO-23AB. 10 μM each), or ASO-C(20 μM). Exon 23 skipping or cryptic splice site activation (% of total) was quantified and is indicated below each lane.

FIG. 29F shows ASOs induce CFTR-W1282X exon 23 skipping. RT-PCR analysis of exon 23 splicing in CFF16HBEge-W1282X cells treated with ASO-23AB, or ASO-C at indicated concentrations. Exon 23 skipping and cryptic splice site activation was quantified and is indicated below each lane. β-actin is a control for RNA expression in all experiments.

FIG. 30A shows ASO-induced exon skipping increases membrane conductance of a CFTR-W1282X cell line. Conductance (Gt) traces of a CFF16HBEge-W1282X clonal cell line selected for high resistance (CFF16HBEge-W1282X-SCC: 3F2). Cells were transfected with vehicle (black line), ASO-C(grey line), or ASO-23AB (grey lines) at indicated concentrations. Cells were pre-treated with DMSO (solid line) or VX-445+VX-661 (dashed lines).

FIG. 30B shows ASO-induced exon skipping increases membrane conductance of a CFTR-W1282X cell line. Average area under the curve (AUC) of the conductance trace (FIG. 30A) was quantified for the forskolin+VX-770 test period for each treatment group. Error bars are ±SEM. One-way ANOVA: Dunnett's multiple comparison test to vehicle+DMSO. ** p<0.01. *** p<0.001. N=3 except for 10 UM ASO-23AB where N=2.

FIG. 30C shows ASO-induced exon skipping increases membrane conductance of a CFTR-W1282X cell line. RT-PCR analysis of exon 23 splicing in CFF16HBEge-W1282X cells in (FIG. 30A). β-actin is a control for RNA expression.

FIG. 30D shows ASO-induced exon skipping increases membrane conductance of a CFTR-W1282X cell line. Quantification of exon 23 skipping (% of total) in (FIG. 30C). Error bars are ±SEM. One-way ANOVA: Dunnett's multiple comparison test to vehicle. * p<0.05. *** p<0.001. N=3 except for 10 UM ASO-23AB where N=2.

FIG. 30E shows ASO-induced exon skipping increases membrane conductance of a CFTR-W1282X cell line. The calculated area under the curve, shown in (FIG. 30B) correlated with exon 23 skipping (%), shown in (FIG. 30D) (simple linear regression).

FIG. 31A shows ASO treatment induces exon 23 skipping, stabilizes CFTR mRNA, and rescues CFTR function in primary human bronchial epithelial (hBE) cells isolated from a patient homozygous for CFTR-W1282X. Equivalent current (leq) traces of primary hBE cells from a homozygous CFTR-W1282X CF donor. Cells were transfected with vehicle, ASO-C, or ASO-23AB (320 μM total) and pre-treated with DMSO, C18, or VX-445+VX-661.

FIG. 31B shows ASO treatment induces exon 23 skipping, stabilizes CFTR mRNA, and rescues CFTR function in primary human bronchial epithelial (hBE) cells isolated from a patient homozygous for CFTR-W1282X. Average AUC of the current traces (FIG. 31A) was quantified for the forskolin or forskolin+VX-770 test periods for each treatment group. Error bars are ±SEM. Two-way ANOVA: Dunnett's multiple comparison test to DMSO within treatment groups. ####p<0.01. Two-way ANOVA: Dunnett's multiple comparison test to vehicle within treatment groups. **** p<0.0001. N=4 except C18 treatment where N=3.

FIG. 31C shows ASO treatment induces exon 23 skipping, stabilizes CFTR mRNA, and rescues CFTR function in primary human bronchial epithelial (hBE) cells isolated from a patient homozygous for CFTR-W1282X. RT-PCR analysis of exon 23 splicing in cells from (FIG. 31A). β-actin is a control for RNA expression.

FIG. 31D shows ASO treatment induces exon 23 skipping, stabilizes CFTR mRNA, and rescues CFTR function in primary human bronchial epithelial (hBE) cells isolated from a patient homozygous for CFTR-W1282X. Quantification of exon 23 skipping in (FIG. 31C). Error bars are +SEM. One-way ANOVA: Dunnett's multiple comparison test. **** p<0.0001. N=4.

FIG. 31E shows ASO treatment induces exon 23 skipping, stabilizes CFTR mRNA, and rescues CFTR function in primary human bronchial epithelial (hBE) cells isolated from a patient homozygous for CFTR-W1282X. RT-PCR analysis of CFTR mRNA expression (exons 11-14) from cells analyzed in (FIG. 31A) compared to hBE cells from a non-CF donor.

FIG. 31F shows ASO treatment induces exon 23 skipping, stabilizes CFTR mRNA, and rescues CFTR function in primary human bronchial epithelial (hBE) cells isolated from a patient homozygous for CFTR-W1282X. RT-qPCR analyses of total CFTR mRNA (exon 11-12) from cells analyzed in (FIG. 31A) compared to hBE cells from a non-CF donor. Error bars are ±SEM. One-way ANOVA: Tukey's multiple comparison test, **** p<0.0001. N=4.

FIG. 31G shows ASO treatment induces exon 23 skipping, stabilizes CFTR mRNA, and rescues CFTR function in primary human bronchial epithelial (hBE) cells isolated from a patient homozygous for CFTR-W1282X. Immunoblot analysis of CFTR protein isolated from cells in (FIG. 31A). Protein from a non-CF donor and a CF donor homozygous for F508del is also shown. SNRPB2 is a loading control.

FIG. 31H shows ASO treatment induces exon 23 skipping, stabilizes CFTR mRNA, and rescues CFTR function in primary human bronchial epithelial (hBE) cells isolated from a patient homozygous for CFTR-W1282X. Quantification of the Total CFTR (B+C Bands)/SNRP2 normalized to vehicle+DMSO shown in (FIG. 31G). Error bars are ±SEM. One-way ANOVA: Dunnett's multiple comparison test to vehicle+DMSO, *p<0.05. **** p<0.0001. N=3.

FIG. 32A shows ASO treatment does not affect modulator activity in hBE cells isolated from a CF patient compound heterozygous for CFTR-W1282X and CFTR-F508del. Equivalent current (Ieq) traces of primary hBE cells isolated from a CF donor heterozygous for CFTR-W1282X and CFTR-F508del. Cells were transfected with vehicle, ASO-C, or ASO-23AB (320 UM total). Cells were pre-treated with DMSO, C18, or VX-445+VX-661.

FIG. 32B shows ASO treatment does not affect modulator activity in hBE cells isolated from a CF patient compound heterozygous for CFTR-W1282X and CFTR-F508del. Average AUC of the current traces (FIG. 32A) was quantified for the forskolin+VX-770 test periods for each treatment group. Error bars are ±SEM. Two-way ANOVA: Dunnett's multiple comparison test to DMSO within treatment groups, #p<0.05. ####p<0.01. Two-way ANOVA: Dunnett's multiple comparison test to vehicle within treatment groups, ns=p>0.05. N=3.

FIG. 32C shows ASO treatment does not affect modulator activity in hBE cells isolated from a CF patient compound heterozygous for CFTR-W1282X and CFTR-F508del. RT-PCR analysis of exon 23 splicing in hBE CFTR-W1282X/F508del cells in (FIG. 32A). β-actin is a control for RNA expression.

FIG. 32D shows ASO treatment does not affect modulator activity in hBE cells isolated from a CF patient compound heterozygous for CFTR-W1282X and CFTR-F508del. Quantification of exon 23 skipping in (FIG. 32C). Error bars are ±SEM. One-way ANOVA: Dunnett's multiple comparison test. ** p<0.01. N=3.

FIG. 32E shows ASO treatment does not affect modulator activity in hBE cells isolated from a CF patient compound heterozygous for CFTR-W1282X and CFTR-F508del. RT-PCR analysis of non-F508del CFTR mRNA expression (exons 11-14) from cells analyzed in (FIG. 32A) compared to hBE cells from a non-CF donor.

FIG. 32F shows ASO treatment does not affect modulator activity in hBE cells isolated from a CF patient compound heterozygous for CFTR-W1282X and CFTR-F508del. RT-qPCR analyses of total CFTR mRNA from non-F508del alleles (exon 11-12) in cells analyzed in (FIG. 32A) compared to hBE cells from a non-CF donor. Error bars are ±SEM. One-way ANOVA: Tukey's multiple comparison test. **** p<0.0001. N=4, one outlier identified and removed with ROUT outlier analysis (Q=5%) in WT.

FIG. 32G shows ASO treatment does not affect modulator activity in hBE cells isolated from a CF patient compound heterozygous for CFTR-W1282X and CFTR-F508del. RT-PCR analysis of F508del CFTR mRNA expression (exons 11-14) from cells analyzed in (FIG. 32A) compared to hBE cells from a non-CF donor.

FIG. 32H shows ASO treatment does not affect modulator activity in hBE cells isolated from a CF patient compound heterozygous for CFTR-W1282X and CFTR-F508del. RT-qPCR analyses of total CFTR mRNA from F508del alleles (exon 11-12) in cells analyzed in (FIG. 32A) compared to hBE cells from a non-CF donor. Error bars are ±SEM. One-way ANOVA: Tukey's multiple comparison test, ns=p>0.05. N=4.

FIG. 33A shows ASO-induced exon 23 exclusion has partial allele specificity for CFTR-W1282X. RT-qPCR analyses of allele-specific mRNA expression isolated from compound heterozygous hBE cells shown in FIGS. 32F&H. Total CFTR mRNA expression from the CFTR-W1282X allele was normalized to mRNA expression from the F508del allele for each treatment group. Error bars are ±SEM. One-way ANOVA: Tukey's multiple comparison test, *p<0.05. N=2.

FIG. 33B shows ASO-induced exon 23 exclusion has partial allele specificity for CFTR-W1282X. Allele specific RT-PCR analysis of ASO-induced exon 23 skipping in hBE cells from a donor heterozygous shown in FIG. 32A. Allele specific transcripts were amplified from exons 11-25 (primers: 11WT-25 [W1282X] or 11ΔF-25 [F508del]). Exon 23 skipping from each allele was analyzed using nested exon 22-24 primers. Exon 23 skipping was quantified (% of total) and is shown below each lane.

FIG. 33C shows ASO-induced exon 23 exclusion has partial allele specificity for CFTR-W1282X. Comparison of calculated SR protein binding sites (ESEfinder, Cold Spring Harbor Laboratory) between WT exon 23 (top) and exon 23 containing the CFTR-W1282X mutation (bottom). Differences are indicated by *.

FIG. 33D shows ASO-induced exon 23 exclusion has partial allele specificity for CFTR-W1282X. RT-PCR analysis of CFTR exon 23 skipping in hBE cells from various donors treated with vehicle. ASO-C, or ASO-23AB (320 μM). Genotype of each donor is indicated. Exon 23 skipping was quantified and is indicated below each lane. β-actin is a control for RNA expression.

FIG. 34 shows the potential of ASO treatments to treat CFTR splicing mutations. Analysis of total number of patients with CFTR splicing mutations in the CFTR exons they effect. CFTR exons capable of in-frame skipping are indicated in grey.

FIG. 35A-35C show that systematic functional analysis of CFTR isoforms lacking symmetrical exons. (FIG. 35A) Schematic of CFTR protein in the cell membrane with protein domains aligned to CFTR exons. Symmetric exons are depicted in green. (FIG. 35B) Quantification of terminating variants found in each CFTR exon from pwCF registered in the CFTR2 database. Green bars represent variants in symmetric exons. (FIG. 35C) Measurement of chloride conductance in cells expressing CFTR or isoforms lacking individual symmetrical exons. Average area under the curve (AUC) was quantified for the forskolin or forskolin+potentiator(s) test periods from conductance traces measured in FRT cells, which lack endogenous CFTR, transfected with empty vector (−), CFTR-F508del. CFTR-WT, or each CFTR exon deletion construct. Cells were pre-treated with vehicle (DMSO), or VX-445+VX-661 (hashed bars) for 24 hours. Potentiators were added after forskolin addition during analysis (VX-770, gray bars, or VX-770+Asp-11, blue bars). Error bars are ±SEM; replicates are DMSO n=4. VX-770 n=3. VX-770+Asp-11 n=1. VX-770+VX-661+VX-445 n=3. VX-770+Asp-11+VX-661+VX-445 n=1 for all except: empty vector n=13.13.7.13.7:45 n=5.3.2.2.2:Δ13 n=7.6.4.6.4: Δ22 n=3.4.3.4.3: Δ23 n=8.9.8.9.8: Δ24 n=5.6.5.6.5: Δ25/26 n=3 (all): ΔF508 n=9.7.7.7.7: WT n=16.16.7.16.7 respectively; two-way ANOVA with Dunnett's multiple comparison test compared to DMSO treatment within groups; ** P<0.01. *** P<0.001, and **** P<0.0001.

FIG. 36A-36E show that antisense oligonucleotides induce skipping of symmetrical exons. (FIG. 36A) Schematic of ASOs aligned to each CFTR exon targeted for skipping. (FIG. 36B) RT-PCR quantification of exon skipping [A/(full-length+cryptic+A)]*100 for each ASO (15 μM). (FIG. 36C-FIG. 36E) (left) RT-PCR analysis of RNA isolated from T84 cells transfected with vehicle (veh), control ASO (C. 120μM), or ASO-23A+ASO-23B (FIG. 36C) ASO-24-3 (FIG. 36D) or ASO-26-4+ASO-25-2 or ASO-25-EX (FIG. 36E) at indicated doses. (right) The percent of transcripts with exon skipping was plotted in relation to ASO dose.

FIG. 37A-37D show that ASO treatment induces exon skipping and rescues CFTR function in primary human bronchial epithelial (hBE) cells isolated from pwCF when used with HEMTs. (FIG. 37A) (left) Equivalent current (Ieq) traces of primary hBE cells from CF donors expressing CFTR-W1282X. The genotype of each donor is indicated on the left. Cells were transfected with ASO-C(80 μM, black), or ASO-23AB (40 μM each, green) and treated with vehicle (DMSO, solid lines) or VX-445+VX-661 (dashed lines) for 24 hours before functional analysis. Forskolin, potentiator [VX-770 (diamond) or VX-770+Asp-11 (star)], and bumetanide additions are indicated. (right) Average AUC of the current traces was quantified for the forskolin or forskolin+potentiator(s) test periods for each treatment group. Error bars are ±SEM; two-way ANOVA with Tukey's multiple comparison test within groups to assess ASO effects or Sidak's multiple comparison test between groups to assess ASP-11 effects; * P<0.05. ** P<0.01. *** P<0.001, and **** P<0.0001. (FIG. 37B) (left) RT-PCR analysis of exon 23 splicing in hBE cells indicated to the left. (right) Quantification of the percent of mRNA with exon 23 skipping for each treated donor. Error bars are ±SEM; one-way ANOVA with Tukey's multiple comparisons test; **** P<0.0001. (FIG. 37C) (left) Equivalent current (leq) traces of primary hBE cells from a CF donor compound heterozygous for CFTR-N1303K and CFTR-F508del. Cells were transfected with ASO-C(40-160 μM, black), or ASO-24-3 (40-160 μM, blue) and treated as in FIG. 37A. (right) Average AUC of the current trace was quantified for the forskolin or forskolin+potentiator(s) test periods for each treatment group. Error bars are ±SEM; two-way ANOVA with Tukey's multiple comparisons test within groups to assess ASO effects or Šídák's multiple comparison test between groups to assess ASP-11 effects; * P<0.05 and *** P<0.001. (FIG. 37D) (left) RT-PCR analysis of exon 24 splicing in hBE cells. (right) Quantification of exon 24 skipping. Error bars are ±SEM; one-way ANOVA with Tukey's multiple comparisons test: **** P<0.0001.

FIG. 38A-38C show that an ASO cocktail induces multi-exon skipping of CFTR exons 25 and 26 in primary human bronchial epithelial (hBE) cells isolated from pwCF. (FIG. 38A) (left) Equivalent current (leq) traces of primary hBE cells from a CF donor compound heterozygous for CFTR-F508del and CFTR-E1371X. Cells were transfected with ASO-C(40-240 M, black), ASO-25 (40-120 μM, pink), or ASO-25+ASO-26 (40-120 μM each, purple) and treated with vehicle (DMSO, solid lines) or VX-445+VX-661 (dashed lines) for 24 hours. Forskolin, potentiator [VX-770 (diamond) or VX-770+ASP-11 (star)], and bumetanide additions are indicated. (right) Average AUC of the current traces was quantified for the forskolin or forskolin+potentiator(s) test periods for each treatment group. Error bars are ±SEM two-way ANOVA with Tukey's multiple comparison test within groups to assess ASO effects or Šídák's multiple comparison test between groups to assess ASP-11 effects; * P<0.05. ** P<0.01. *** P<0.001, and **** P<0.0001. (FIG. 38B) (left) RT-PCR analysis of exon 25 or exon 25/26 splicing in hBE cells from A (right) Quantification of exon 25 or 25/26 skipping. Error bars are +SEM; two-way ANOVA with Tukey's multiple comparisons test ** P<0.01. *** P<0.001, and **** P<0.0001. (FIG. 38C) RT-qPCR analyses of total (left) CFTR-E1371X mRNA (exon 11-12) or (right) CFTR-F508del mRNA (exon 11-12) from treated hBE cells compared to RNA from a non-CF or homozygous CFTR-F508del donor. Error bars are ±SEM; one-way ANOVA with Dunnet's multiple comparisons test to ASO-C; ** P<0.01 and **** P<0.0001.

FIG. 39A and FIG. 39B show that ASO treatment induces exon 22 skipping but does not rescue CFTR function in primary human bronchial epithelial (hBE) cells isolated from a CF individual expressing terminating variants. (FIG. 39A) (left) Equivalent current (Ieq) traces of primary hBE cells from CF donors expressing at least one nonsense variant in exon 22. The genotype of each donor is indicated at the left. Cells were transfected with ASO-C(40-160 μM, black), or ASO-22-1 (40-160 μM, orange) and treated with vehicle (DMSO, solid lines) or VX-445+VX-661 (dashed lines) for 24 hours. Forskolin, potentiator [VX-770 (diamond) or VX-770+Asp-11 (star)], and bumetanide additions are indicated. (right) Average AUC of the current traces was quantified for the forskolin or forskolin+potentiator(s) test periods for each treatment group. Error bars are ±SEM; within groups to assess ASO effects or Šídák's multiple comparison test between groups to assess ASP-11 effects; *P<0.05. (FIG. 39B) (left) RT-PCR analysis of exon 22 splicing from cells in FIG. 39A. (right) Quantification of exon 22 skipping. Error bars are ±SEM; one-way ANOVA with Tukey's multiple comparisons test; * P<0.05 and *** P<0.001.

FIG. 40A and FIG. 40B show that expression of CFTR exon deletion constructs. (FIG. 40A) RT-PCR analysis of CFTR RNA collected from FRT cells transfected with each CFTR exon deletion construct, compared to CFTR-WT. (FIG. 40B) (top) Immunoblot analysis of CFTR protein, bands C and B, isolated from FRT cells stably transfected with empty vector. CFTR-F508del, CFTR-WT, or each CFTR exon deletion construct. Cells were treated with DMSO or VX-445+VX-661. β-actin was used as a loading control. (middle) Total CFTR protein (band C+band B/β-actin was quantified. (bottom) The ratio of CFTR (band C/band B) isoforms was quantified. Replicates are n=2 and experimental groups are connected by a line.

FIG. 40C shows a representative current traces of CFTR constructs analyzed in FIG. 1C.

FIG. 41 shows the cellular localization of CFTR and the exon skipped isoforms. Immunofluorescent images of FRT cells transfected with empty vector, CFTR-WT. CFTR-F508del. CFTR-A22. CFTR-Δ23. CFTR-424, or CFTR-425/26. Cells were incubated with a CFTR-specific monoclonal antibody (green) and a rabbit Claudin polyclonal antibody (magenta). Regions of interest at the cell membrane were enlarged to show co-localization of CFTR at the cell membrane (white).

FIG. 42A and FIG. 42B show spliced-switching ASOs induce skipping of symmetrical CFTR exons. (FIG. 42A) RT-PCR analysis of T84 cells transfected with vehicle, control ASO (ASO-C), or CFTR exon targeting ASOs. Amplicons representing cryptically spliced CFTR is indicated by * and expected exon skipping for each ASO is labelled with A on the right side of the gel. Quantification of gels are shown in FIG. 36B. ASOs that induced the highest level of skipping are highlighted in green. (FIG. 42B) RT-PCR analysis of T84 cells transfected with vehicle, control ASO (ASO-C), or CFTR exon 25 and 26 targeting ASOs. Exon skipping is quantified as [A/(full-length+4) |*100.

FIG. 43A and FIG. 43B show that ASO treatment induces CFTR-W1282X exon skipping but does not increase function above approved HEMTs when expressed with CFTR-F508del (FIG. 43A) (left) Equivalent current (Ieq) traces of primary hBE cells from CF donors. The genotype of each donor is indicated on the left. Cells were transfected with ASO-C(80 μM, black), or ASO-23AB (40 μM each, green) and treated with vehicle (DMSO, solid lines) or VX-445+VX-661 (dashed lines) for 24 hours. Forskolin, potentiator [VX-770 (diamond) or VX-770+Asp-11 (star)], and bumetanide additions are indicated. (right) Average AUC of the current traces was quantified for the forskolin or forskolin+potentiator(s) test periods for each treatment group. Error bars are ±SEM two-way ANOVA with Tukey's multiple comparison within groups to assess ASO effects or Sidák's multiple comparison test between groups to assess ASP-11 effects; * P<0.05. ** P<0.01. (FIG. 43B) (left) RT-PCR analysis of exon 23 splicing in hBE cells from A. (right) Quantification of percent exon 23 skipping for each treated donor. Error bars are ±SEM; one-way ANOVA with Tukey's multiple comparisons test: *P<0.05. ** P<0.01. **** P<0.0001.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present disclosure.

### DETAILED DESCRIPTION OF THE DISCLOSURE

The present disclosure relates to general compounds and methods to treat cystic fibrosis in subjects using antisense oligonucleotides (ASOs) that induce specific pre-mRNA splicing events in CFTR gene transcripts that result in mRNAs that code for proteins that fully or partially restore the function of CFTR (i.e., resulting in increased levels of correctly localized CFTR protein at the plasma membrane and with increased function). Frameshift and nonsense mutations pose a major problem for disease therapeutic development. For example, some ASOs can base-pair with the target RNA and correct aberrant splicing caused by mutations, and other ASOs can induce skipping of exons with mutations that cause open reading frame-shifts. In such instances, skipping of the mutated exon using ASOs can restore the reading frame and generate an mRNA that codes for a CFTR isoform with partial function. Eliminating these mutations from the mRNA by inducing exon skipping is a relatively unexplored treatment approach, though it has shown promise for some diseases. As shown herein, elimination of a common stop mutation associated with cystic fibrosis by inducing skipping of the exon it is located in, results in a restoration of the open reading frame and recovers CFTR protein function in a manner expected to be therapeutic in CF patients who don't currently have effective treatment options. These results are an important advancement for the cystic fibrosis community but also have implications for other diseases where terminating mutations are responsible for dysfunction.

The CFTR gene encodes a member of the ATP-binding cassette (ABC) transporter superfamily. ABC proteins transport various molecules across extra—and intra-cellular membranes. ABC genes are divided into seven distinct subfamilies (ABC1, MDR/TAP, MRP, ALD, OABP, GCN20, White). The CFTR protein is a member of the MRP subfamily that is involved in multi-drug resistance. The encoded protein functions as a chloride channel and controls the regulation of other transport pathways. Mutations in the CFTR gene are associated with the autosomal recessive disorders cystic fibrosis and congenital bilateral aplasia of the vas deferens. Alternatively spliced transcript variants have been described, many of which result from mutations in this gene.

Human (Homo sapiens) cystic fibrosis transmembrane conductance regulator is located on chromosome 7:117,465,784-117,715,971 (forward strand; SEQ ID NO: 130). The gene is 6132 bp mRNA (Gene ID: 1080; Official Symbol: CFTR; Official Full Name: cystic fibrosis transmembrane conductance regulator) and is assigned NCBI Reference Sequence: NM_000492.3 (SEQ ID NO: 145); ACCESSION: NM_000492; Ensembl: ENSG00000001626; HPRD: 03883; MIM: 602421; and Vega: OTTHUMG00000023076. CFTR is also known as: CF; MRP7; ABC35; ABCC7; CFTR/MRP; TNR-CFTR; dJ760C5.1. Human CFTR protein is assigned NCBI Reference Sequence: NP_000483.3 (1480 aa; SEQ ID NO: 146).

The mouse (Mus musculus) cystic fibrosis transmembrane conductance regulator is located on chromosome 6:18170687-18322768 (SEQ ID NO: 147). The mouse CFTR gene is 6305 bp (Gene ID: 12638; Official Symbol: Cftr; Official Full Name: cystic fibrosis transmembrane conductance regulator), and is also known as: Abcc7; AW495489; ATP-binding cassette sub-family C member 7; ATP-binding cassette transporter sub-family C member 7; ATP-binding cassette, subfamily c, member 7; cAMP-dependent chloride channel; channel conductance-controlling ATPase; cystic fibrosis transmembrane conductance regulator homolog cystic fibrosis transmembrane conductance regulator homolog; ATP-binding cassette, subfamily c, member 7. The mouse CFTR gene has been assigned NCBI Reference Sequence: NM_021050.2 (SEQ ID NO: 148), and Ensembl: ENSMUSG00000041301. The mouse CFTR protein is assigned NCBI Reference Sequence: NP_066388.1 (1476 aa; SEQ ID NO: 149).

Antisense compounds, (e.g. antisense oligonucleotides (ASOs)) have been used to modulate target nucleic acids. Antisense compounds comprising a variety of chemical modifications and motifs have been reported. In certain instances, such compounds are useful as research tools, diagnostic reagents, and as therapeutic agents. In certain instances, antisense compounds have been shown to modulate protein expression by binding to a target messenger RNA (mRNA) encoding the protein. In certain instances, such binding of an antisense compound to its target mRNA results in cleavage of the mRNA. Antisense compounds that modulate processing of a pre-mRNA have also been reported. Such antisense compounds alter splicing, interfere with polyadenlyation or prevent formation of the 5′-cap of a pre-mRNA.

Pre-mRNA splicing involves the precise and accurate removal of introns from the pre-messenger RNA and the ligation of exons together after intron removal to generate the mature mRNA which serves as the template for protein translation. Pre-mRNA splicing is a two-step reaction carried out by a spliceosome complex comprising protein and small RNA components which recognize conserved sequence elements within the introns and exons of the RNA. Recognition of these sequence elements, including the 5′ splice site, 3′ splice site and branch point sequence, is the primary mechanism directing the correct removal of introns.

Splicing requires direct base-pairing between small nuclear RNA (snRNA) components of the spliceosome and the splice site nucleotides of the mRNA. This interaction can be easily disrupted by gene mutations or by artificial blocking using short oligonucleotides complementary to the RNA. Such so called antisense oligonucleotides (ASOs), when designed to be complementary to a splice sites, will compete for base-pairing with the snRNAs, thereby blocking an essential step in splicing at the site. In this way, antisense oligonucleotides can potently block unwanted splicing or redirect splicing to alternative splice sites, and can result in mRNAs that code for proteins that fully or partially restore the function to target transcripts.

For example, ASOs can target the 2789+5G>A mutation in intron 16 of the CFTR gene that causes cystic fibrosis. This mutation has been observed in 521 patients with cystic fibrosis. Because aberrant splicing of exon 16 due to the mutation is the cause of cystic fibrosis in patients with this mutation, improving splicing using antisense oligonucleotides to interfere with the deleterious effects of the mutation, can have a therapeutic benefit to the patients. In a non-limiting example, an antisense oligonucleotide that targets the 2789+5G>A mutation of the CFTR gene that causes cystic fibrosis can be SEQ ID NO: 97.

In another non-limiting example, antisense oligonucleotides can target the 3849+10kbC->T mutation in intron 19 of the CFTR gene. This mutation has been observed in 496 patients, and in 1.100 patients in CFTR2 database. The 3849+10kbC>T mutation creates a cryptic splice site that results in an aberrant mRNA that does not produce CFTR protein and antisense oligonucleotides targeted to the region of intron 19 surrounding and encompassing this mutation can potentially block splicing to this cryptic splice site. In a non-limiting example, an antisense oligonucleotide that targets the 3849+10kbC>T mutation of the CFTR gene that causes cystic fibrosis can be SEQ ID NO:150.

In yet another non-limiting example, antisense oligonucleotides can target the 3272-26A->G mutation of the CFTR gene that causes cystic fibrosis. This mutation is found in 186 patients. The 3272-26A>G mutation creates a cryptic splice site that results in an aberrant mRNA that does not produce CFTR protein. Antisense oligonucleotides targeted to the region of surrounding and encompassing this mutation can potentially block splicing to this cryptic splice site. In a non-limiting example, an antisense oligonucleotide that targets the 3272-26A->G mutation of the CFTR gene that causes cystic fibrosis can be SEQ ID NO: 114.

In another non-limiting example, antisense oligonucleotides can target exon skipping in exons that have nonsense mutations. For example, skipping of exon 4, exon 23, exon 24, exon 25, or exon 26 all can result in an mRNA transcript that is in-frame so that translation will continue to the natural stop-codon (i.e., mutations such as CFTR 621+1G>T and CFTR 406G>T). Exons 4, 23, 24, 25 and 26 have a number of different patient nonsense mutations that cause cystic fibrosis and any of these can be treated by ASOs that induce exon skipping of the exons that house nonsense mutations to correct the reading frame and allow translation through to the natural termination codon.

In yet other non-limiting examples, 70-90% of all Cystic fibrosis (CF) patients have a mutation in exon 11 (deltaF508) which can be targeted by ASO 11-6 (SEQ ID NO.: 91). Five percent of CF patients have a splice site mutation in intron 16 which can be targeted and corrected by ASO 16-8 (SEQ ID NO.: 102); 2.8% of CF patients have a nonsense mutation in exon 23 which can be targeted for skipping and frame-shift correction using ASO 23-4 (SEQ ID NO.: 126) and ASO-23-3 (SEQ ID NO.: 125); 0.33% of CF patients have a nonsense mutation in exon 24 which can be targeted for skipping and frame-shift correction using ASO 24-1, 24-2, 24-3 (SEQ ID NO.: 127, 128, 129; respectively); CF mutation databases indicate that nonsense and splicing mutations in and around exon 4 are common and can be targeted for gene expression correction either by splicing redirection or frame-shift correction using ASO 4-1 (SEQ ID NO.: 65); and CF causing nonsense mutations in exons 2, 5, 7, 9, 10, 13, 20 and 22 are also commonly annotated in the Human Gene Mutation Database and can be targeted by ASOs 2-4, 5-1,7-4, 9-1, 11-6, 13-1, 15-1, 20-2, 22-1 (SEQ ID NO.: 64, 71, 76, 78, 91, 92, 94, 111, 116; respectively). Around 0.21% of CF patients have a terminating mutation in exons 25 and 26 and can be targeted for correction via exon skipping, including the E1373X mutation, for example by ASO-25-EX (SEQ ID NO.: 177) or ASO-25-2 (SEQ ID NO.: 175) in combination with ASO-26-4 (SEQ ID NO.: 181).

In certain embodiments, this disclosure provides ASOs and the use of them in combination, such that one ASO blocks splicing of exon 25 (e.g. SEQ ID Nos: 174-177) and another ASO blocks splicing of exon 26 (e.g. SEQ ID Nos: 178-181). The ASOs together, when used in combination, result in exon 25 and 26 skipping, which results in a CFTR mRNA spliced isoform that is in the same open reading frame as the full-length CFTR-encoding mRNA. This CFTR delta-exon 25/26 isoform encodes a CFTR protein with partial function. Treatment of cystic fibrosis cells with mutations in either exon 25 or exon 26 with two separate ASOs can result in removal of the mutation from the mRNA, stabilization of the mRNA, and production of a therapeutic CFTR protein isoform. In addition, treatment with either ASO alone can induce only skipping of exon 25 or exon 26, which, when used in cells with a CFTR mutation in the respective targeted exon, can stabilize the mRNA and thereby increase CFTR protein production.

Additionally, CFTR gene mutations that introduce premature termination codons account for ˜10% of cystic fibrosis cases. This mutation type is associated with a severe form of the disease, typically a consequence of low CFTR mRNA levels resulting from degradation by nonsense mediated mRNA decay (NMD), and production of a truncated, non-functional CFTR protein. Current therapeutics for CF are less effective in patients with these types of mutations, likely due to the instability of the mRNA and lack of the natural C-terminal portion of the protein. Antisense technology is a promising therapeutic strategy for these types of mutations that has not been widely explored for the disease. Splice-switching antisense oligonucleotides (ASOs) can be designed to modify gene expression by directly modulating pre-mRNA splicing. ASO-mediated skipping of exons to restore the open reading frame of RNA with frameshift mutations or premature termination codons (PTC) has been successfully applied to treat a number of disorders. This approach of treating disease associated with PTCs eliminates the PTC by inducing skipping of the exon encoding the variant and results in retention of the proper reading frame.

In one aspect, one or more modified oligonucleotides that are complementary to an equal-length portion of a target region of a cystic fibrosis transmembrane conductance regulator (CFTR) transcript can be used to treat CFRT. In certain embodiments, the one or more oligonucleotides bind a target region that is about 25 nucleobases upstream and/or about 25 downstream of the exon to be skipped. In some embodiments, the targeted exon is exon 2, exon 4, exon 5, exon 7, exon 9, exon 10, exon 11, exon 13, exon 15, exon 16, exon 20, exon 22, intron 22, exon 23, exon 24, exon 25, or exon 26 of human CFTR. In certain embodiments, the two or more oligonucleotides bind a target region is within: (a) nucleobase 198,888 and nucleobase 199,094 of SEQ ID NO: 130; or (b) nucleobase 199,705 and nucleobase 199,796 of SEQ ID NO: 130. In some embodiments, the target region is within nucleobase 198,888 and nucleobase 199,094 of SEQ ID NO: 130, and each of the two or more modified oligonucleotides is selected from the group consisting of SEQ ID NOs: 174-177. In some embodiments, the target region is within nucleobase 199,705 and nucleobase 199.796 of SEQ ID NO: 130, and each of the two or more modified oligonucleotides is selected from the group consisting of SEQ ID NOS: 178-181.

For example, ASOs can target the W1282X mutation (the 5th most common mutation in CFTR) in exon 23 of the CFTR gene that causes cystic fibrosis. This nonsense mutation truncates CFTR at amino acid 1281 (CFTR1281), removing ˜60% of the nucleotide binding domain 2 (NBD2) but retaining most of the full-length protein (1281 vs 1480 amino acids). The truncated protein may have processing and/or gating defects as an increase in channel function can be achieved in W1282X-CFTR cells by potentiator and corrector treatment. Stabilizing W1282X-CFTR by elimination of the PTC using an ASO that induces the exclusion of exon 23 during the process of pre-mRNA splicing is a potential approach. Skipping of exon 23 results in the production of a CFTR mRNA with an intact open-reading frame with a deletion of the 52 amino acids encoded by exon 23. In a non-limiting example, two or more antisense oligonucleotides that target splicing of exon 23 can be used to treat CFTR patients with the W1282X mutation. In another non-limiting example, antisense oligonucleotides that target splicing of exon 23 can be SEQ ID NO:123-126.

In certain embodiments, the ASO compositions disclosed herein are used to treat CFTR patients with one or more Class I mutations. Class I mutations result in the presence of premature termination codons (PTCs). These “stop” codons do not allow the CFTR protein to be produced, leading to an absence of CFTR protein at the epithelial membrane. In some embodiments, CFTR mutations suitable for treatment with the ASO compositions disclosed herein comprise one or more of W1282X, L1254X, S1255X, L1258FfsX7, R1283M, 11295FfsX, S1297FfsX, G1298WfsX, N1303TfsX, Q1313X, E92X, Q98X, R104EfsX, 1105SfsX, Y109GfsX, Y122X, 1148LfsX, F157X, G542X, N1303K, R553X, 621+1G>T, 1717-1G>A, 1507del, R1162X, or E831X, K1250RfsX9, 3856delC. L1243X, G1247X, S1248X, 3876delA, 3878delG, 3886insA, 3889dupT, 3892delTT. 3898insC, 3905delT, 3905insT, L1253X, E1266X, 3940delG, 3944delGT, W1274X, 3959delC, 3960-3961delA, 3967deITT, Q1280X, Q1281X, G1287X, Q1291X, 4006delA, c.3883_3884insG, 4010del4, 4015delA, c.3889delT, 4016insT, 4022insT, c.3893delG, K1302X, 4040delA, 4040dupA, 4048insCC, Y1307X, E1308X, Q1309X, W1310X, 4064-4065delinsAATATG, Q1313X, W1316X, 4089ins4, 4089delA, 4108delT, 4120delCA, Q1330X, c.3999delG, c.4028delG, 4160insG, 4165dclGT, p.S1347PfsX13, 4168delCTAAGCC, c.4036dupC, 4168delCTAAG, 4172delGC, 4177delG. K1351X. 4197_4198delCT, 4203TAG->AA. 4209TGTT->AA, c.4078delG. 4218insT. 4222delG. 4237-4242delinsAGAA. E1371X. 4259dcl5, 4271dclC. Y1381X. Q1382X. 4279insA, c.4163_4167delTAAAA, Q1390X. 4301dclA, c.4187delC. 4326delTC. 4332delTG, C1400X. E1401X, E1409X. C1410X, Q1411X, and Q1412X.

TABLE S1
Total CF-causing PTC variants in CFTR exons 23-26 reported in CFTR2. For each variant,
the total number of alleles is indicated as well as the total number of homozygous, compound
heterozygous with F508del, and compound heterozygous with non-F508del genotypes.

| exon | Variant legacy name | cDNA | alleles | homozygous | F508del | other |
|---|---|---|---|---|---|---|
| 23 | 3856delC | c.3724del | 1 | 0 | 1 | 0 |
| | L1243X | c.3728T > A | 1 | 0 | 0 | 1 |
| | G1247X | c.3739G > T | 2 | 0 | 0 | 2 |
| | S1248X | c.3743C > A \| c.3743C > G | 5 | 0 | 3 | 2 |
| | 3876delA | c.3744del | 172 | 18 | 65 | 71 |
| | 3878delG | c.3747del | 20 | 1 | 11 | 7 |
| | 3886insA | c.3754dup | 1 | 0 | 1 | 0 |
| | 3889dupT | c.3758dup | 1 | 0 | 1 | 0 |
| | 3892delTT | c.3760_3761del | 1 | 0 | 0 | 1 |
| | L1254X | c.3761T > A \| c.3761T > G | 35 | 0 | 29 | 6 |
| | S1255X | c.3764C > A | 40 | 2 | 12 | 24 |
| | 3898insC | c.3767dup | 1 | 0 | 1 | 0 |
| | 3905delT | c.3773del | 1 | 0 | 1 | 0 |
| | 3905insT | c.3773dup | 330 | 4 | 221 | 101 |
| | L1253X | c.3773T > A | 1 | 0 | 0 | 1 |
| | E1266X | c.3796G > T | 3 | 0 | 1 | 2 |
| | 3940delG | c.3808del | 4 | 0 | 1 | 3 |
| | 3944delGT | c.3816_3817del | 26 | 0 | 14 | 12 |
| | W1274X | c.3822G > A | 8 | 0 | 7 | 1 |
| | 3959delC | c.3827del | 1 | 0 | 1 | 0 |
| | 3960-3961delA | c.3829del | 1 | 0 | 0 | 1 |
| | 3967delTT | c.3835_3836del | 2 | 0 | 2 | 0 |
| | Q1280X | c.3838C > T | 3 | 0 | 2 | 1 |
| | Q1281X | c.3841C > T | 6 | 0 | 2 | 4 |
| | W1282X | c.3845G > A \| c.3846G > A | 2500 | 238 | 1079 | 945 |
| | G1287X | c.3859G > T | 2 | 0 | 0 | 2 |
| | Q1291X | c.3871C > T | 5 | 1 | 2 | 1 |
| | W1282X; R1283M | c.[3846G > A; 3848G > T] | 7 | 0 | 6 | 1 |
| 24 | 4006delA | c.3876del | 1 | 0 | 0 | 1 |
| | c.3883_3884insG | c.3883_3884insG | 4 | 2 | 0 | 0 |
| | 4010del4 | c.3883_3886del | 18 | 2 | 5 | 9 |
| | 4015delA | c.3883del | 36 | 0 | 17 | 19 |
| | c.3889delT | c.3889del | 1 | 0 | 0 | 1 |
| | 4016insT | c.3889dup | 156 | 4 | 75 | 73 |
| | 4022insT | c.3891dup | 11 | 0 | 7 | 4 |
| | c.3893delG | c.3893del | 1 | 0 | 1 | 0 |
| | K1302X | c.3904A > T | 4 | 1 | 0 | 2 |
| | 4040delA | c.3908del | 31 | 0 | 17 | 14 |
| | 4040dupA | c.3908dup | 2 | 0 | 1 | 1 |
| | 4048insCC | c.3917_3918dup | 1 | 0 | 0 | 1 |
| | Y1307X | c.3921T > A | 2 | 0 | 0 | 2 |
| | E1308X | c.3922G > T | 4 | 0 | 2 | 2 |
| | Q1309X | c.3925C > T | 4 | 0 | 1 | 3 |
| | W1310X | c.3929G > A \| c.3930G > A | 30 | 2 | 17 | 9 |
| | 4064- | c.3932_3933delinsAATATG | 2 | 0 | 1 | 1 |
| | 4065delinsAATATG | | | | | |
| | Q1313X | c.3937C > T | 50 | 1 | 30 | 18 |
| | W1316X | c.3947G > A | 2 | 0 | 1 | 1 |
| | 4089ins4 | c.3957_3958insAGGG | 1 | 0 | 1 | 0 |
| | 4089delA | c.3957del | 1 | 0 | 1 | 0 |
| 25 | 4108delT | c.3976del | 2 | 0 | 2 | 0 |
| | 4120delCA | c.3988_3989del | 1 | 0 | 1 | 0 |
| | Q1330X | c.3988C > T | 10 | 0 | 7 | 3 |
| | c.3999delG | c.3999del | 1 | 0 | 1 | 0 |
| | c.4028delG | c.4028del | 2 | 0 | 2 | 0 |
| | 4160insG | c.4028dup | 2 | 0 | 1 | 1 |
| | 4165delGT | c.4033_4034del | 1 | 0 | 0 | 1 |
| | p.S1347PfsX13 | c.4035_4038dup | 7 | 1 | 5 | 0 |
| | 4168delCTAAGCC | c.4036_4042del | 3 | 0 | 3 | 0 |
| | c.4036dupC | c.4036dup | 1 | 0 | 1 | 0 |
| | 4168delCTAAG | c.4037_4041del | 1 | 0 | 0 | 1 |
| | 4172delGC | c.4040_4041del | 1 | 0 | 0 | 1 |
| | 4177delG | c.4046del | 2 | 0 | 1 | 1 |
| | K1351X | c.4051A > T | 1 | 0 | 1 | 0 |
| | 4197_4198delCT | c.4065_4066del | 1 | 0 | 0 | 1 |
| | 4203TAG -> AA | c.4071_4073delinsAA | 1 | 0 | 1 | 0 |
| | 4209TGTT -> AA | c.4077_4080delinsAA | 49 | 1 | 35 | 12 |
| | c.4078delG | c.4078del | 1 | 0 | 0 | 1 |
| | 4218insT | c.4086dup | 9 | 0 | 5 | 4 |
| | 4222delG | c.4090del | 2 | 0 | 2 | 0 |
| | 4237- | c.4105_4110delinsAGAA | 1 | 0 | 1 | 0 |
| | 4242delinsAGAA | | | | | |
| | E1371X | c.4111G > T | 28 | 0 | 16 | 12 |
| | 4259de15 | c.4127_4131del | 5 | 0 | 1 | 4 |
| 26 | 4271delC | c.4139del | 4 | 0 | 4 | 0 |
| | Y1381X | c.4143C > A \| c.4143C > G | 2 | 0 | 1 | 1 |
| | Q1382X | c.4144C > T | 12 | 0 | 8 | 4 |
| | 4279insA | c.4147dup | 6 | 0 | 5 | 1 |
| | c.4163_4167delTAAAA | c.4163_4167del | 1 | 0 | 1 | 0 |
| | Q1390X | c.4168C > T | 3 | 1 | 1 | 0 |
| | 4301delA | c.4170del | 1 | 0 | 1 | 0 |
| | c.4187delC | c.4187del | 1 | 0 | 0 | 1 |
| | 4326delTC | c.4197_4198del | 32 | 0 | 21 | 11 |
| | 4332delTG | c.4200_4201del | 2 | 0 | 0 | 2 |
| | C1400X | c.4200T > A | 1 | 0 | 1 | 0 |
| | E1401X | c.4201G > T | 2 | 0 | 0 | 2 |
| | E1409X | c.4225G > T | 1 | 0 | 1 | 0 |
| | C1410X | c.4230C > A | 1 | 0 | 0 | 1 |
| | Q1411X | c.4231C > T | 18 | 3 | 7 | 5 |
| | Q1412X | c.4234C > T | 12 | 0 | 5 | 7 |
| total | | | 3773 | 282 | 1782 | 1427 |

In certain embodiments, the ASO compositions disclosed herein are used to treat CFTR patients with one or more mutations to the CFTR gene. In some embodiments, CFTR mutations suitable for treatment with the ASO compositions disclosed herein comprise one or more of E56K, P67L, R74W, D110E, D110H, R117C, R117H, G178R, E193K, L206W, R347H, R352Q, A455E, S549N, S549R, G551D, G551S, D570G, D579G, S945L, S977F, F1052V, K1060T, A1067T, G1069R, R1070Q, R1070W, F1074L, D1152H, G1244E, S1251N, S1255P, D1270N, G1349D, G85E, D1152H, R334W, R560T, L206W, P67L, M1101K, M470V, L997F, F508del, 711+3A>G, 2789+5G>A, 3272-26A>G, and 3849+10kbC>T.

In some embodiments, CFTR mutations suitable for treatment with the ASO compositions disclosed herein comprise one or more of 3856delC, L1243X, G1247X, S1248X, 3876delA, 3878delG, 3886insA, 3889dupT, 3892delTT, 3898insC, 3905delT, 3905insT, L1253X, E1266X, 3940delG, 3944delGT, W1274X, 3959delC, 3960-3961delA, 3967deITT, Q1280X, Q1281X, G1287X, and Q1291X.

In some embodiments, CFTR mutations suitable for treatment with the ASO compositions disclosed herein comprise one or more of 4006delA, c.3883_3884insG, 4010del4, 4015delA, c.3889delT, 4016insT, 4022insT, c.3893delG, K1302X, 4040delA, 4040dupA, 4048insCC, Y1307X, E1308X, Q1309X, W1310X, 4064-4065delinsAATATG, Q1313X, W1316X, 4089ins4, and 4089delA.

In some embodiments, CFTR mutations suitable for treatment with the ASO compositions disclosed herein comprise one or more of 4108delT, 4120delCA, Q1330X, c.3999delG, c.4028delG, 4160insG, 4165delGT, p.S1347PfsX13, 4168delCTAAGCC, c.4036dupC, 4168delCTAAG, 4172delGC, 4177delG, K1351X, 4197_4198delCT, 4203TAG->AA, 4209TGTT->AA, c.4078delG, 4218insT, 4222delG, 4237-4242delinsAGAA, E1371X, and 4259del5.

In some embodiments, CFTR mutations suitable for treatment with the ASO compositions disclosed herein comprise one or more of 4271delC, Y1381X, Q1382X, 4279insA, c.4163_4167delTAAAA, Q1390X, 4301delA, c.4187delC, 4326delTC, 4332delTG, C1400X, E1401X, E1409X, C1410X, Q1411X, and Q1412X.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “antisense compound” or “antisense oligonucleotide (ASO)” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid. As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “chemical modification” means a chemical difference in a compound when compared to a naturally occurring counterpart. In reference to an oligonucleotide, chemical modification does not include differences only in nucleobase sequence. Chemical modifications of oligonucleotides include nucleoside modifications (including sugar moiety modifications and nucleobase modifications) and internucleoside linkage modifications.

As used herein, “sugar moiety” means a naturally occurring sugar moiety or a modified sugar moiety of a nucleoside.

As used herein, “modified sugar moiety” means a substituted sugar moiety, a bicyclic or tricyclic sugar moiety, or a sugar surrogate.

As used herein, “substituted sugar moiety” means a furanosyl comprising at least one substituent group that differs from that of a naturally occurring sugar moiety. Substituted sugar moieties include, but are not limited to, furanosyls comprising substituents at the 2′-position, the 3′-position, the 5′-position and/or the 4′-position.

As used herein, “2′-substituted sugar moiety” means a furanosyl comprising a substituent at the 2′-position other than —H or —OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH₂CH₂OCH₃.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl.

As used herein, the term “sugar surrogate” means a structure that does not comprise a furanosyl and that is capable of replacing the naturally occurring sugar moiety of a nucleoside, such that the resulting nucleoside is capable of: (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, “nucleotide” means a nucleoside further comprising a phosphate linking group.

As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “heterocyclic base” or “heterocyclic nucleobase” means a nucleobase comprising a heterocyclic structure.

As used herein, the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′—CH(CH₃)-0-2′bridge.

As used herein, “locked nucleic acid nucleoside” or “LNA” means a nucleoside comprising a bicyclic sugar moiety comprising a 4′—CH₂-0-2′bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′—H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more substructures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” means an atom or group of atoms bound to an oligonucleotide or oligomeric compound. In general, conjugate groups modify one or more properties of the compound to which they are attached, including, but not limited to, pharmacodynamic, pharmacokinetic, binding, absorption, cellular distribution, cellular uptake, charge and/or clearance properties.

As used herein. “conjugate linking group” means any atom or group of atoms used to attach a conjugate to an oligonucleotide or oligomeric compound.

As used herein, “detectable and/or measureable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, the term “about” encompasses insubstantial variations, such as values within a standard margin of error of measurement (e.g., SEM) of a stated value. For example, the term “about” as used herein when referring to a measurable value such as a parameter, an amount, a temporal duration, can encompass variations of +/−10% or less, +/−5% or less, or +/−1% or less or less of and from the specified value. Designation of a range of values includes all integers within or defining the range, and all subranges defined by integers within the range. As used herein, statistical significance means p≤0.05.

As used herein. “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound hybridizes.

As used herein, “mRNA” means an RNA molecule that encodes a protein.

As used herein, “pre-mRNA” means an RNA transcript that has not been fully processed into mRNA. Pre-RNA includes one or more intron.

As used herein, “transcript” means an RNA molecule transcribed from DNA. Transcripts include, but are not limited to mRNA, pre-mRNA, and partially processed RNA.

As used herein. “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein. “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “motif “means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein. “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified.” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleoside have “the same type of modification.” even though the DNA nucleoside is unmodified. Such nucleosides having the same type modification may comprise different nucleobases.

As used herein. “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

### Certain Motifs

In certain embodiments, the present invention provides oligomeric compounds comprising oligonucleotides. In certain embodiments, such oligonucleotides comprise one or more chemical modification. In certain embodiments, chemically modified oligonucleotides comprise one or more modified nucleosides. In certain embodiments, chemically modified oligonucleotides comprise one or more modified nucleosides comprising modified sugars. In certain embodiments, chemically modified oligonucleotides comprise one or more modified nucleosides comprising one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

### Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer). In certain embodiments, oligonucleotides comprise 2′-MOE modified nucleosides in the wings and 2′-F modified nucleosides in the gap.

In certain embodiments, oligonucleotides are fully modified. In certain such embodiments, oligonucleotides are uniformly modified. In certain embodiments, oligonucleotides are uniform 2′-MOE. In certain embodiments, oligonucleotides are uniform 2′-F. In certain embodiments, oligonucleotides are uniform morpholino. In certain embodiments, oligonucleotides are uniform BNA. In certain embodiments, oligonucleotides are uniform LNA. In certain embodiments, oligonucleotides are uniform cEt.

In certain embodiments, oligonucleotides comprise a uniformly modified region and additional nucleosides that are unmodified or differently modified. In certain embodiments, the uniformly modified region is at least 5, 10, 15, 20 or 25 nucleosides in length. In certain embodiments, the uniform region is a 2′-MOE region. In certain embodiments, the uniform region is a 2′-F region. In certain embodiments, the uniform region is a morpholino region. In certain embodiments, the uniform region is a BNA region. In certain embodiments, the uniform region is a LNA region. In certain embodiments, the uniform region is a cEt region.

In certain embodiments, the oligonucleotide does not comprise more than 4 contiguous unmodified 2′-deoxynucleosides. In certain circumstances, antisense oligonucleotides comprising more than 4 contiguous 2′-deoxynucleosides activate RNase H, resulting in cleavage of the target RNA. In certain embodiments, such cleavage is avoided by not having more than 4 contiguous 2′-deoxynucleosides, for example, where alteration of splicing and not cleavage of a target RNA is desired.

Certain internucleoside Linkage Motifs

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for sugar modification motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The sugar modification motif is independently selected, so such oligonucleotides having a gapped internucleoside linkage motif may or may not have a gapped sugar modification motif and if it does have a gapped sugar motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

### Certain Nucleobase Modification Motifs

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

### Certain Overall Lengths

In certain embodiments, the present invention provides oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

One of skill in the art will appreciate that certain lengths may not be possible for certain motifs. For example: a gapmer having a 5′-wing region consisting of four nucleotides, a gap consisting of at least six nucleotides, and a 3′-wing region consisting of three nucleotides cannot have an overall length less than 13 nucleotides. Thus, one would understand that the lower length limit is 13 and that the limit of 10 in “10-20” has no effect in that embodiment. Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range. For example, an oligonucleotide consisting of 20-25 linked nucleosides comprising a 5′-wing consisting of 5 linked nucleosides; a 3′-wing consisting of 5 linked nucleosides and a central gap consisting of 10 linked nucleosides (5+5+10=20) may have up to 5 nucleosides that are not part of the 5′-wing, the 3′-wing, or the gap (before reaching the overall length limitation of 25). Such additional nucleosides may be 5′ of the 5′-wing and/or 3′ of the 3′ wing.

### Certain Oligonucleotides

In certain embodiments, oligonucleotides of the present invention are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. Herein if a description of an oligonucleotide or oligomeric compound is silent with respect to one or more parameter, such parameter is not limited. Thus, an oligomeric compound described only as having a gapmer sugar motif without further description may have any length, internucleoside linkage motif, and nucleobase modification motif. Unless otherwise indicated, all chemical modifications are independent of nucleobase sequence.

### Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Bchmoaras et al., EMBO J., 1991, 10, 1111-1118; Kabanov et al., FEBS Lett, 1990, 259, 327-330; Svinarchuk et al., Biochimie, 1993, 75, 49-54), a phospholipid, e.g., di-hexadecyl-rac-glycerol or tricthyl-ammonium 1.2-di-O-hexadecyl-rac-glycero-3-H-phosphonate (Manoharan et al., Tetrahedron Lett., 1995, 36:3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36:3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C₂-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′end of an oligomeric compound, but before one or more terminal group nucleosides. In certain embodiments, conjugate groups are placed within a terminal group.

In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

### Antisense Compounds

In certain embodiments, oligomeric compounds of the present invention are antisense compounds. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present invention provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

In certain embodiments antisense compounds and antisense oligonucleotides comprise single-strand compounds. In certain embodiments antisense compounds and antisense oligonucleotides comprise double-strand compounds.

### Pharmaceutical Compositions

In certain embodiments, the present invention provides pharmaceutical compositions comprising one or more antisense compound. The pharmaceutical composition may comprise a cocktail of antisense compounds, wherein the cocktail comprises 2, 3, 4, 5, 6, 7, 8, 9, 10 or more antisense compounds. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, antisense compounds may be admixed with pharmaceutically acceptable active and/or inert substances for the preparation of pharmaceutical compositions or formulations.

In certain embodiments, the antisense compounds may be combined with a cystic fibrosis transmembrane conductance regulator (CFTR) modulator or a CFTR modulator therapy. CFTR modulator therapies are designed to correct the malfunctioning protein made by the CFTR gene. In some embodiments, CFTR modulators can be ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof. In some embodiments, a CFRT modulator therapy can comprise Kalydeco® (ivacaftor), Orkambi® (lumacaftor and ivacaftor), Symdeko® (tezacaftor and ivacaftor), Trikafta® (elexacaftor and tezacaftor and ivacaftor), or VX-661+VX-561+VX121. In some embodiments, a CFTR modulator dose can comprise 10, 20, 25, 30, 50, 75, 100, 150, 200, 250, 300, 350, 400, 450, 500, 550, or 600 mg once or twice daily.

Compositions and methods for the formulation of pharmaceutical compositions depend on a number of criteria, including, but not limited to, route of administration, extent of disease, or dose to be administered.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active antisense oligomeric compound.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono—or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide (DMSO) are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80TM and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80TM: the fraction size of polyethylene glycol may be varied: other biocompatible polymers may replace polyethylene glycol, e.g., polyvinyl pyrrolidone; and other sugars or polysaccharides may substitute for dextrose.

In certain embodiments, a pharmaceutical composition provided herein is prepared for oral administration. In certain embodiments, pharmaceutical compositions are prepared for buccal administration.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition provided herein comprises an oligonucleotide in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more modified oligonucleotide provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, the present invention provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human. In certain embodiments, the animal is a mouse.

In certain embodiments, the present invention provides methods of administering a pharmaceutical composition comprising an oligomeric compound of the present invention to an animal. Suitable administration routes include, but are not limited to, oral, rectal, transmucosal, transdermal, intestinal, enteral, topical, suppository, through inhalation, intrathecal, intracerebroventricular, intraperitoneal, intranasal, intratumoral, and parenteral (e.g., intravenous, intramuscular, intramedullary, and subcutaneous). In certain embodiments, a pharmaceutical composition is prepared for transmucosal administration. In certain of such embodiments penetrants appropriate to the barrier to be permeated are used in the formulation. Such penetrants are generally known in the art. In certain embodiments, pharmaceutical compositions are administered to achieve local rather than systemic exposures. For example, pharmaceutical compositions may be aerosolized and inhaled directly in the area of desired effect (e.g., into the lungs).

In certain embodiments, a pharmaceutical composition is administered to an animal having at least one symptom associated with Cystic Fibrosis. In certain embodiments, such administration results in amelioration of at least one symptom. In certain embodiments, administration of a pharmaceutical composition to an animal results in an increase in functional CFTR protein in a cell. In certain embodiments, the administration of certain antisense oligonucleotides (ASOs) delays the onset of Cystic Fibrosis. In certain embodiments, the administration of certain antisense oligonucleotides prevents the onset of Cystic Fibrosis. In certain embodiments, the administration of certain antisense oligonucleotides rescues cellular phenotype.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′—OH sugar moiety and a thymine base could be described as a DNA having a modified sugar (2′—OH for the natural 2′—H of DNA) or as an RNA having a modified base (thymine(methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence

### Examples

The Examples that follow are illustrative of specific embodiments of the invention, and various uses thereof. They are set forth for explanatory purposes only, and are not to be taken as limiting the invention.

### Methods

Antisense Oligonucleotides (ASOs). ASOs with phosphorodiamidate morpholino (PMO) chemistries were generated by GeneTools LLC and were dissolved in 0.9% saline.

Cell culture and transfection. T84 cells are a human colonic adenocarcinoma cell line and the mouse primary cell line, 208EE, was established from an adult C57BL/6 mouse kidney. ASOs (15 μM final concentration) were transfected into cells using Endo-Porter (GeneTools). RNA was collected 48 hours post-transfection.

RNA isolation and analysis. RNA was isolated from tissue and cells in culture using TRIZOL™ reagent (Life Technologies, Carlsbad, CA) according to the manufacturer's protocol. For human tissue, RNA was isolated and treated with 4 μg of DNase-I (RNase-free) (Life Technologies) followed by reverse transcription with GoScript™ reverse transcription system (Promega, Madison, WI). Radiolabeled and cold PCR was carried out using primers specific for human or mouse CFTR region encompassing the ASO target exon. PCR products were separated by polyacrylamide or agarose gel electrophoresis and bands on gels were quantitated by densitometry analysis using Image J software.

### Example 1: Antisense Oligonucleotides Induce Skipping of Targeted Exons in Murine CFTR gene-derived pre-mRNA

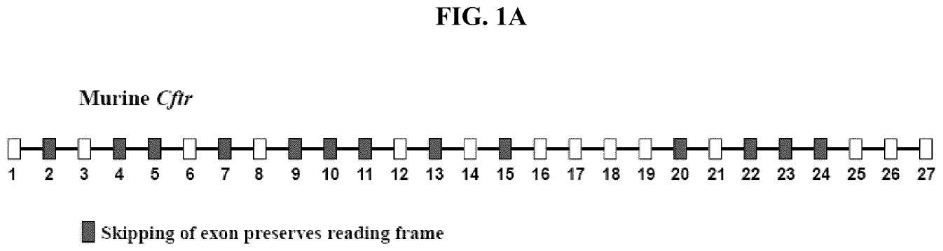

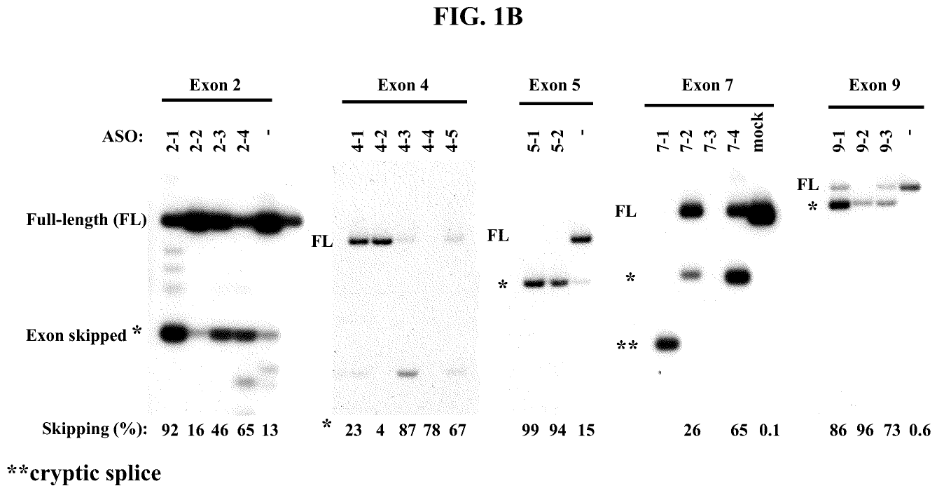

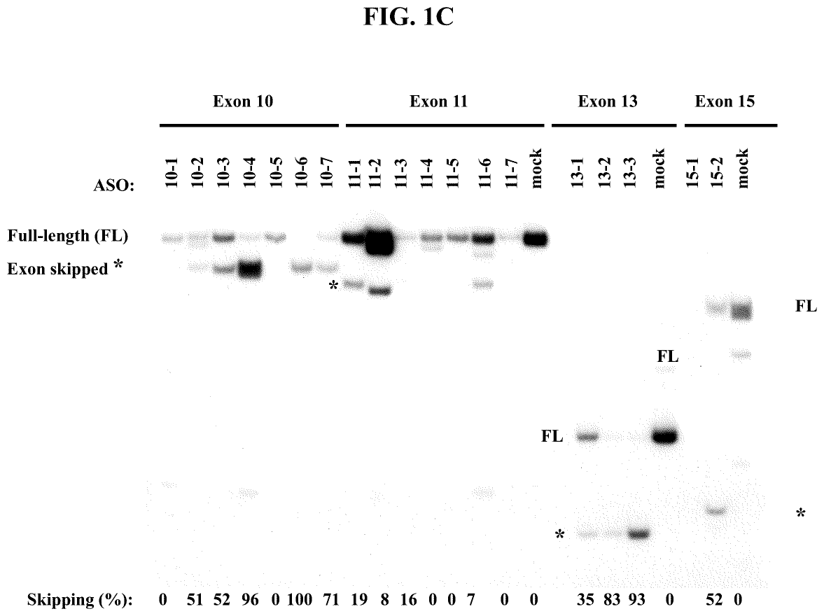

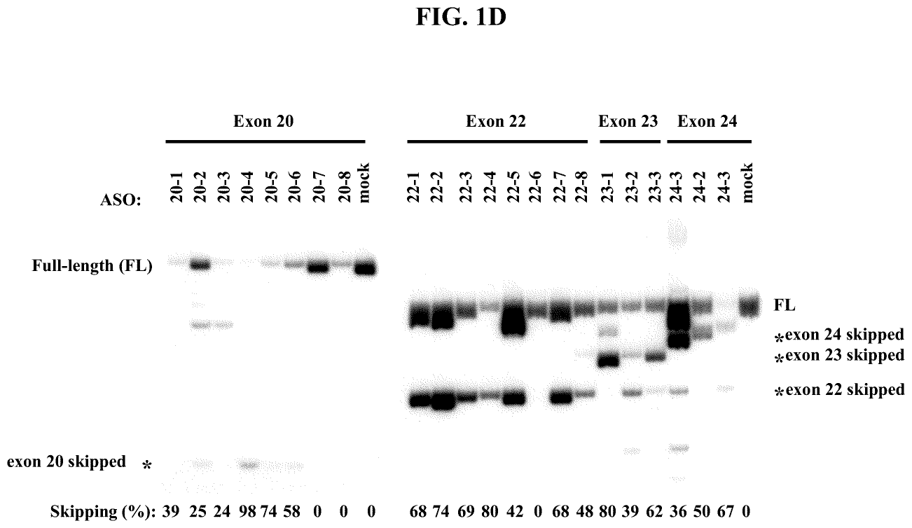

Various ASOs (see Table 1; SEQ ID NOs: 1-60) were tested in the mouse primary cell line, 208EE (which was established from an adult C57BL/6 mouse kidney). ASOs (15 μM final concentration) were transfected into cells using Endo-Porter (GeneTools). FIGS. 1B, 1C and 1D demonstrate that ASOs induce skipping of targeted exons in murine CFTR.

TABLE 1
Antisense oligonucleotides targeting
mouse CFTR induce exon skipping.

| | Target | | % | |
|---|---|---|---|---|
| Name | Exon | Sequence (5′-3′) | skipped * | SEQ ID NO. |
| 2-1 | 2 | GGTCCAGCTAAAAGAGAAGAGGGCA | 92 | SEQ ID NO. 1 |
| 2-2 | 2 | CTTTCCTCAAAATTGGTGTGGTCCA | 16 | SEQ ID NO. 2 |
| 2-3 | 2 | TATGTCTGACAACTCCAAGTGGTGT | 46 | SEQ ID NO. 3 |
| 2-4 | 2 | CTAGTTTTTCAGACAAGTGGTCAGC | 65 | SEQ ID NO. 4 |
| 4-1 | 4 | TTCCTAGCAAGACAGGCTGGACAGC | nd | SEQ ID NO. 5 |
| 4-2 | 4 | ATAGGATGCTATGATTCTTCCTAGC | 23 | SEQ ID NO. 6 |
| 4-3 | 4 | ATAAGCCTATGCCAAGGTAAATGGC | 4 | SEQ ID NO. 7 |
| 4-4 | 4 | TGTCCTGACAATGAAGAGAAGGCAT | 87 | SEQ ID NO. 8 |
| 4-5 | 4 | AATGCGATGAAGGCCAAAAATAGCT | 78 | SEQ ID NO. 9 |
| 4-6 | 4 | TAGCTGTTCTCATCTGCATTCCAAT | 67 | SEQ ID NO. 10 |
| 4-7 | 4 | CATCTTCCAAAAAGTATTACCTTCT | nd | SEQ ID NO. 11 |
| 5-1 | 5 | TTGTTCAGGTTGTTGGAAAGAAGAC | 99 | SEQ ID NO. 12 |
| 5-2 | 5 | ATCAAGAACGCGGCTTGACAACTTT | 94 | SEQ ID NO. 13 |
| 7-1 | 7 | CACGAGTCTTTCATTGATCTTTGCA | 20 | SEQ ID NO. 14 |
| 7-2 | 7 | CTGATTCCCAACAATATGCCTTAAC | 26 | SEQ ID NO. 15 |
| 7-3 | 7 | CAATCATTTTCTCCATCGCTGATTC | 42 | SEQ ID NO. 16 |
| 7-4 | 7 | ATTATGTCAACTTACTCTCTCAAGT | 65 | SEQ ID NO. 17 |
| 9-1 | 9 | GCCTGTGGTCATTAAGTTATACTCC | 86 | SEQ ID NO. 18 |
| 9-2 | 9 | CTCCTCCCAAAATGCTGTTACATTT | 96 | SEQ ID NO. 19 |
| 9-3 | 9 | TATTTAGAAATCTCACCTCCTCCCA | 73 | SEQ ID NO. 20 |
| 10-1 | 10 | CTTTCTCCAGTAATTCCCCAAATCC | 0 | SEQ ID NO. 21 |
| 10-2 | 10 | GTCACCATTGCTTTGTTGTACTTTC | 51 | SEQ ID NO. 22 |
| 10-3 | 10 | CTGAAACTGACATTGTTCTCATCAC | 52 | SEQ ID NO. 23 |
| 10-4 | 10 | AGGATTTCCCACAAGGCAGAGATGA | 96 | SEQ ID NO. 24 |
| 10-5 | 10 | ATAGCCAACATCTCTCCTTTCTCTA | 0 | SEQ ID NO. 25 |
| 10-6 | 10 | CTTTCCTGATCCAGTAGATCCAGTA | 100 | SEQ ID NO. 26 |
| 10-7 | 10 | TTAAAGAGACAGTACCTTTCCTGAT | 71 | SEQ ID NO. 27 |
| 11-1 | 11 | TCCAGTTCTCCCAAAATCAACATCA | 19 | SEQ ID NO. 28 |
| 11-2 | 11 | TGTGCTTAATAATTCCCTCTGAAGC | 8 | SEQ ID NO. 29 |
| 11-3 | 11 | ATTGAGAGCAGAATGAAACTCTTCC | 16 | SEQ ID NO. 30 |
| 11-4 | 11 | GATATTTTCTTTGATAGTACCCGGC | 0 | SEQ ID NO. 31 |
| 11-5 | 11 | ACACTCTTATATCTGTACTCATCAT | 0 | SEQ ID NO. 32 |
| 11-6 | 11 | CTGCTGTAGTTGGCAAGCTTTGACA | 7 | SEQ ID NO. 33 |
| 11-7 | 11 | CATRAATATGCTTACCTGCTGTAGT | 0 | SEQ ID NO. 34 |
| 13-1 | 13 | GGGAATCTAATAGGTACAAATCAGC | 35 | SEQ ID NO. 35 |
| 13-2 | 13 | CAAATCAGCATCTTTATATACTGCT | 83 | SEQ ID NO. 36 |
| 13-3 | 13 | ACTCAGTCATAGAACATACCTTTCA | 93 | SEQ ID NO. 37 |
| 15-1 | 15 | AACAAACATACTTACCTCAACCAGA | 52 | SEQ ID NO. 38 |
| 20-1 | 20 | CCTGCCTGTAAATCATCCCATAGGA | 39 | SEQ ID NO. 39 |
| 20-2 | 20 | CAAGGTGGGTGAAAATTGGACTCCT | 25 | SEQ ID NO. 40 |
| 20-3 | 20 | CGAAGTGTCCAGAGTCCTTTTAAGC | 24 | SEQ ID NO. 41 |
| 20-4 | 20 | CAGAGTTTCAAAGTAAGTCTGGCGT | 98 | SEQ ID NO. 42 |
| 20-5 | 20 | TTGGCAGTGTGCAAATTCAGAGCTT | 74 | SEQ ID NO. 43 |
| 20-6 | 20 | CTATTCTCATTTGGAACCAGCGCAA | 58 | SEQ ID NO. 44 |
| 20-7 | 20 | AGAGGACAAATATCATGTCTATTCT | 0 | SEQ ID NO. 45 |
| 20-8 | 20 | ATGGAGATGAAGGTAACAACAATGA | 0 | SEQ ID NO. 46 |
| 22-1 | 22 | AACTTAAACACTCTGCTCACAGATC | 68 | SEQ ID NO. 47 |
| 22-2 | 22 | CTAAAACGTCAGATGATCCTTCTCT | 74 | SEQ ID NO. 48 |
| 22-3 | 22 | TATCACTTTTCTTCACATGCTCATT | 69 | SEQ ID NO. 49 |
| 22-4 | 22 | ACCATTTCGCCTCCAGAGGGCCAGA | 80 | SEQ ID NO. 50 |
| 22-5 | 22 | CATCCATGTATTTCACAGTAAGGTC | 42 | SEQ ID NO. 51 |
| 22-6 | 22 | ATGTTCTCTAATACGGCATTTCCAT | 0 | SEQ ID NO. 52 |
| 22-7 | 22 | CCTCTGTCCAGGACTTATTGAAAAA | 68 | SEQ ID NO. 53 |
| 22-8 | 22 | GTAATGCTGAAATCTCACCCTCTGT | 48 | SEQ ID NO. 54 |
| 23-1 | 23 | AATTCCATGAGACACCATCAATCTC | 80 | SEQ ID NO. 55 |
| 23-2 | 23 | GTACTTTTTCCTGATCCAGTTCTTC | 39 | SEQ ID NO. 56 |
| 23-3 | 23 | CATTTTTGTGCTCACCTGTGTTATC | 62 | SEQ ID NO. 57 |
| 24-1 | 24 | CATCTTTCCATTTTCCATTGGGATC | 36 | SEQ ID NO. 58 |
| 24-2 | 24 | CTCATCTGCAACTTTCCATATTTCT | 50 | SEQ ID NO. 59 |
| 24-3 | 24 | TATTTGTCATCCTTACCTCATCTGC | 67 | SEQ ID NO. 60 |

* percent of the mRNA transcripts that skip out the targeted exon

### Example 2: Antisense Oligonucleotides Induce Skipping of Targeted Exons in Human CFTR Gene-Derived Pre-mRNA

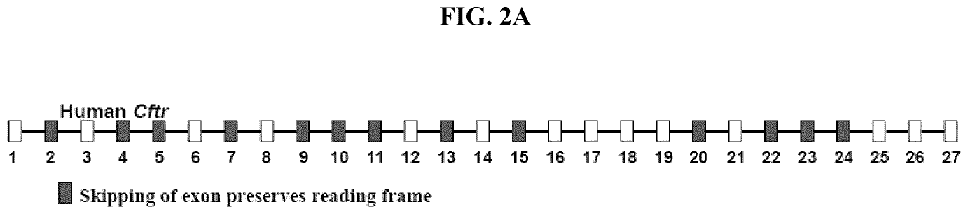

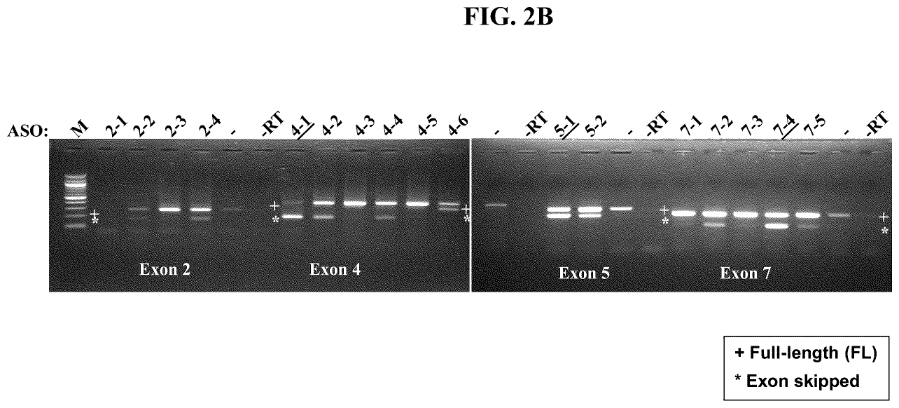

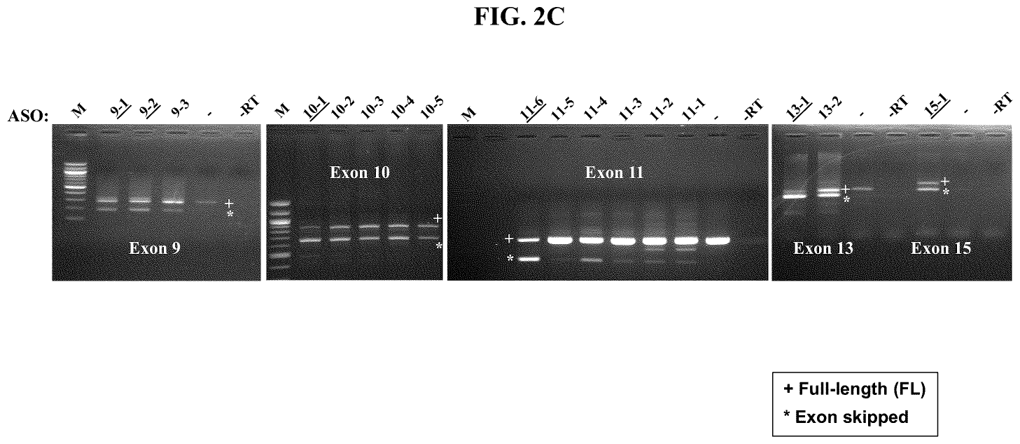

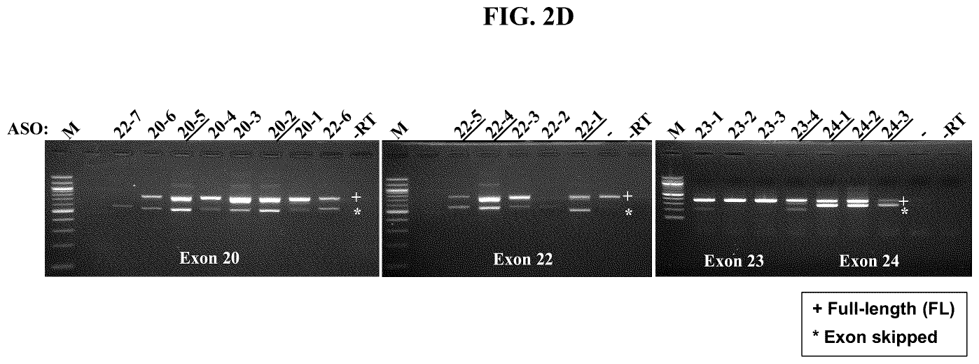

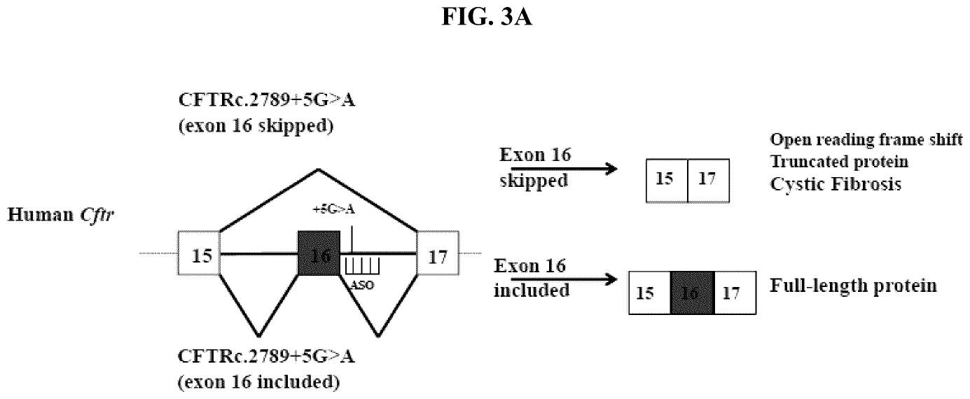

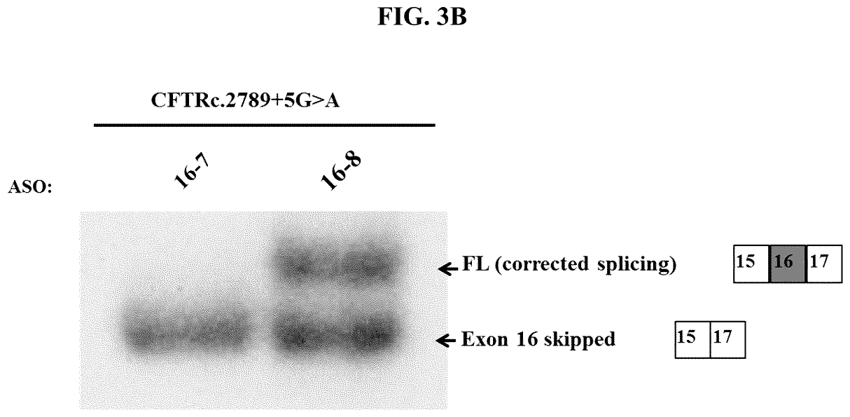

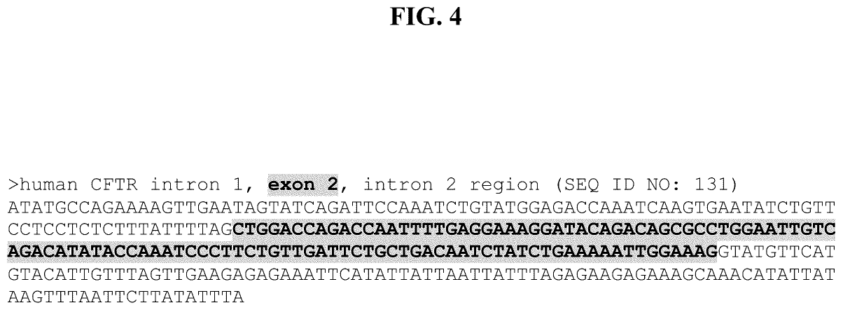

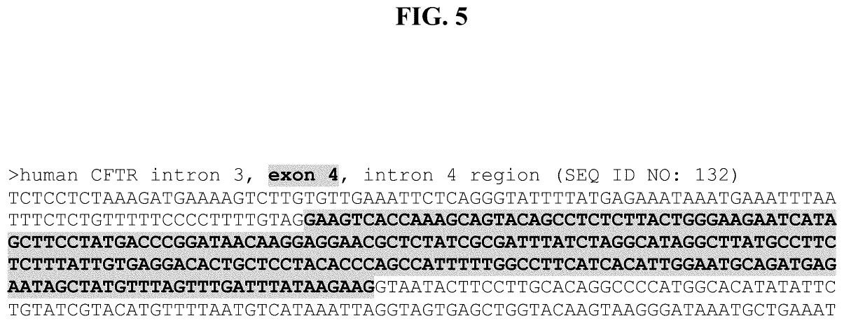

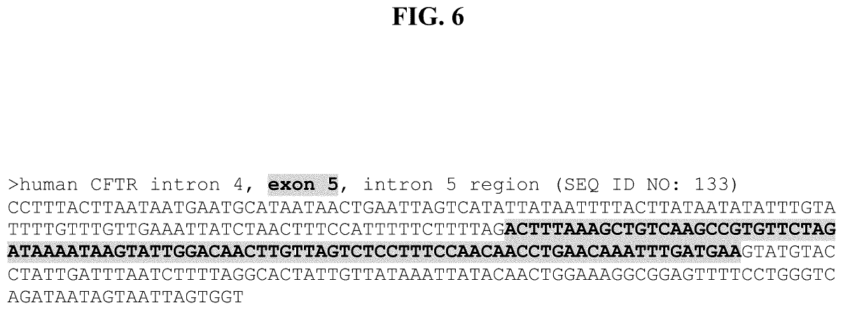

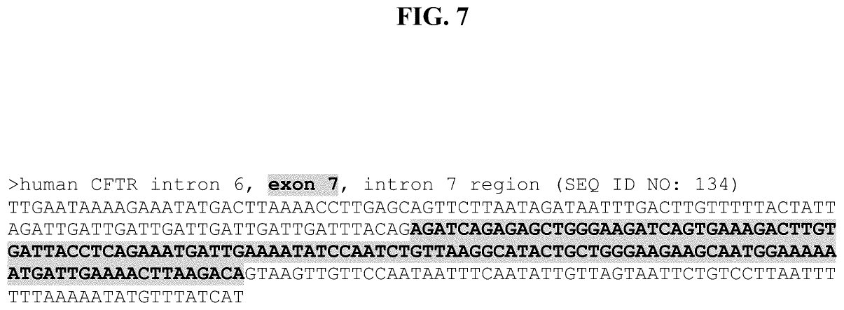

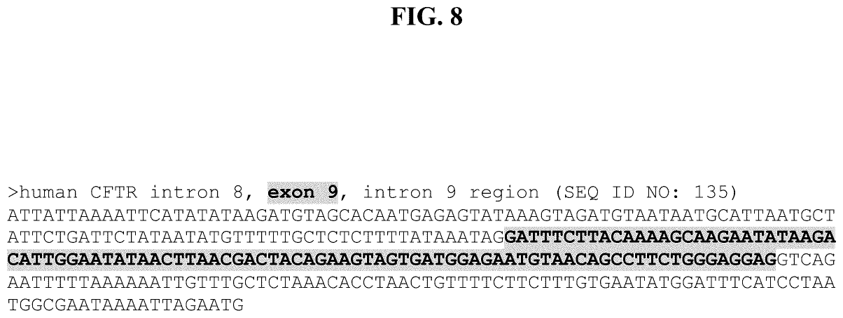

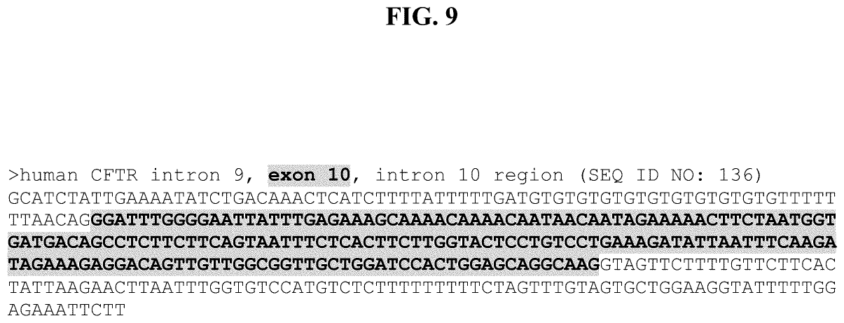

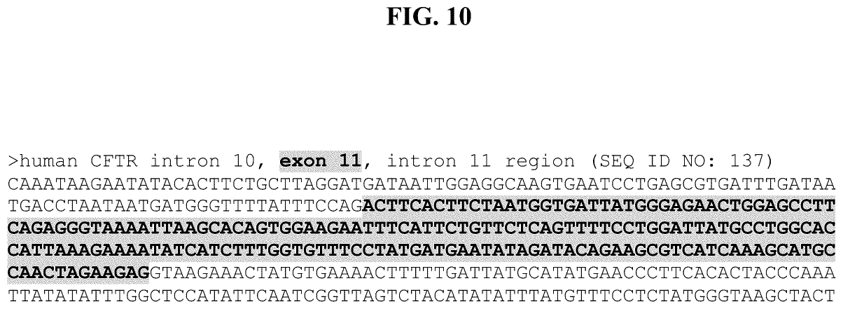

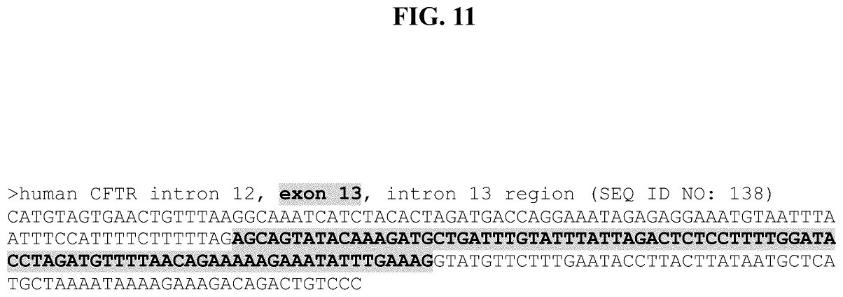

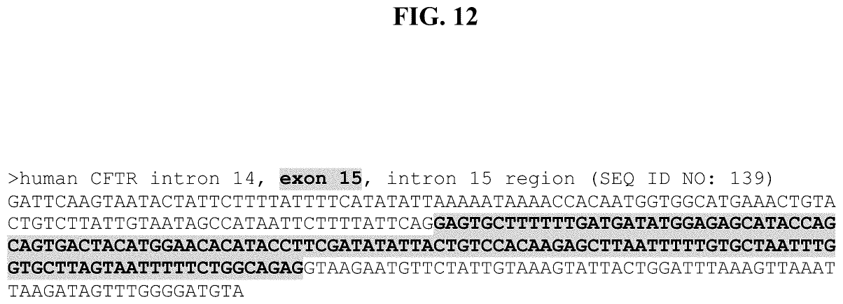

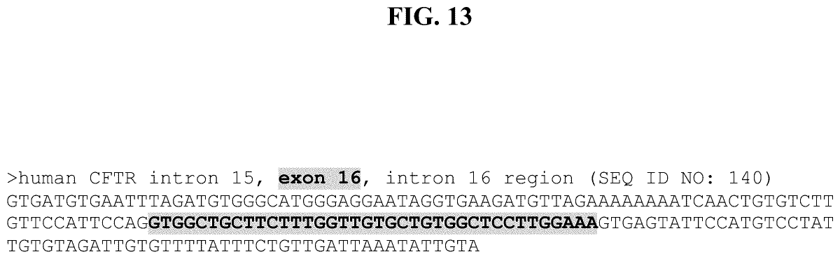

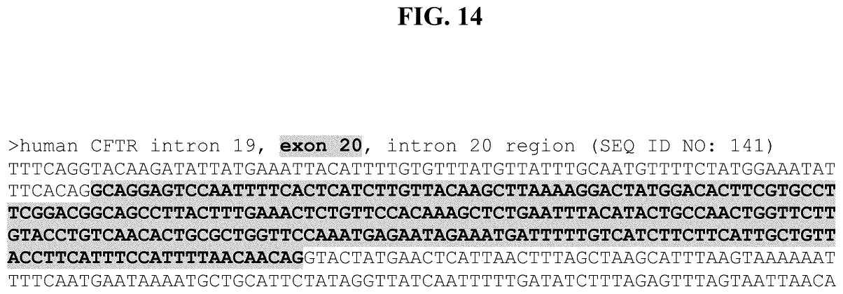

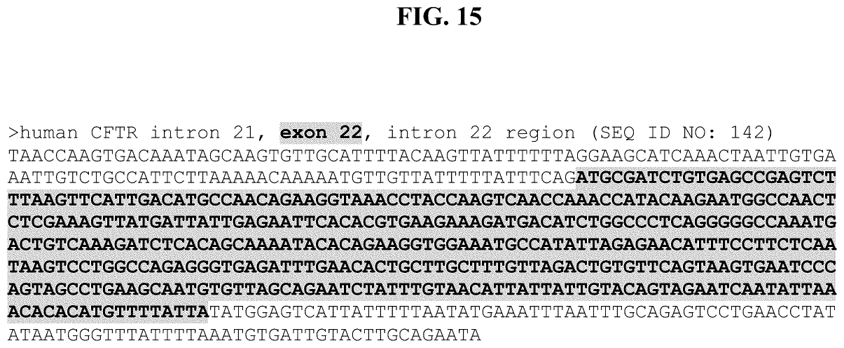

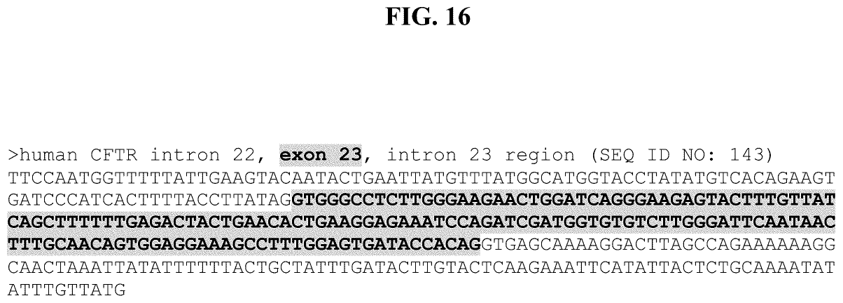

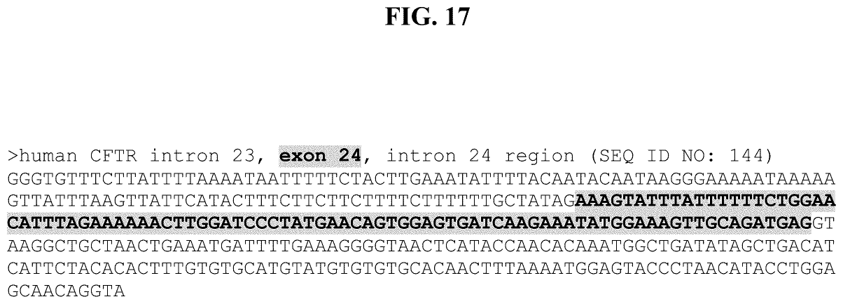

Various ASOs (see Table 2; SEQ ID NOs: 61-129) were tested in the human colonic adenocarcinoma cell line primary cell line, T84. ASOs (15 UM final concentration) were transfected into cells using Endo-Porter (GeneTools). FIGS. 2B, 2C, 2D and FIG. 3 demonstrate that ASOs induce skipping of targeted exons in human CFTR.

TABLE 2
Antisense oligonucleotides targeting
human CFTR induce exon skipping

| | Target | | % | |
|---|---|---|---|---|
| Name | Exon | Sequence (5′-3′) | skipped * | SEQ ID NO. |
| 2-1 | 2 | ATCCTTTCCTCAAAATTGGTCTGGT | 0 | SEQ ID NO. 61 |
| 2-2 | 2 | GTATATGTCTGACAATTCCAGGCGC | 35 | SEQ ID NO. 62 |
| 2-3 | 2 | CAGATAGATTGTCAGCAGAATCAAC | 18 | SEQ ID NO. 63 |
| 2-4 | 2 | GTACATGAACATACCTTTCCAATTT | 37 | SEQ ID NO. 64 |
| 4-1 | 4 | GAGGCTGTACTGCTTTGGTGACTTC | 77 | SEQ ID NO. 65 |
| 4-2 | 4 | GAAGCTATGATTCTTCCCAGTAAGA | 54 | SEQ ID NO. 66 |
| 4-3 | 4 | GTGTAGGAGCAGTGTCCTCACAATA | 0 | SEQ ID NO. 67 |
| 4-4 | 4 | AATGTGATGAAGGCCAAAAATGGCT | 39 | SEQ ID NO. 68 |
| 4-5 | 4 | GCTATTCTCATCTGCATTCCAATGT | 0 | SEQ ID NO. 69 |
| 4-6 | 4 | CCTGTGCAAGGAAGTATTACCTTCT | 0 | SEQ ID NO. 70 |
| 5-1 | 5 | CTAGAACACGGCTTGACAGCTTTAA | 58 | SEQ ID NO. 71 |
| 5-2 | 5 | TGGAAAGGAGACTAACAAGTTGTCC | 42 | SEQ ID NO. 72 |
| 7-1 | 7 | ACTGATCTTCCCAGCTCTCTGATCT | 15 | SEQ ID NO. 73 |
| 7-2 | 7 | ATTTCTGAGGTAATCACAAGTCTTT | 37 | SEQ ID NO. 74 |
| 7-3 | 7 | AGTATGCCTTAACAGATTGGATATT | 28 | SEQ ID NO. 75 |
| 7-4 | 7 | ATTTTTTCCATTGCTTCTTCCCAGC | 44 | SEQ ID NO. 76 |
| 7-5 | 7 | ATTGGAACAACTTACTGTCTTAAGT | 38 | SEQ ID NO. 77 |
| 9-1 | 9 | TCCATCACTACTTCTGTAGTCGTTA | 56 | SEQ ID NO. 78 |
| 9-2 | 9 | CTCCTCCCAGAAGGCTGTTACATTC | 53 | SEQ ID NO. 79 |
| 9-3 | 9 | TTAAAAATTCTGACCTCCTCCCAGA | 33 | SEQ ID NO. 80 |
| 10-1 | 10 | GGCTGTCATCACCATTAGAAGTTTT | 64 | SEQ ID NO. 81 |
| 10-2 | 10 | AATTACTGAAGAAGAGGCTGTCATC | 56 | SEQ ID NO. 82 |
| 10-3 | 10 | TAATATCTTTCAGGACAGGAGTACC | 49 | SEQ ID NO. 83 |
| 10-4 | 10 | GATCCAGCAACCGCCAACAACTGTC | 52 | SEQ ID NO. 84 |
| 10-5 | 10 | AGAACAAAAGAACTACCTTGCCTGC | 47 | SEQ ID NO. 85 |
| 11-1 | 11 | CTCCCATAATCACCATTAGAAGTGA | 2 | SEQ ID NO. 86 |
| 11-2 | 11 | ATTTTACCCTCTGAAGGCTCCAGTT | 2 | SEQ ID NO. 87 |
| 11-3 | 11 | ACAGAATGAAATTCTTCCACTGTGC | 2 | SEQ ID NO. 88 |
| 11-4 | 11 | GTGCCAGGCATAATCCAGGAAAACT | 14 | SEQ ID NO. 89 |
| 11-5 | 11 | ATGCTTTGATGACGCTTCTGTATCT | 2 | SEQ ID NO. 90 |
| 11-6 | 11 | TTTTCACATAGTTTCTTACCTCTTC | 72 | SEQ ID NO. 91 |
| 13-1 | 13 | TCTAGGTATCCAAAAGGAGAGTCTA | 90 | SEQ ID NO. 92 |
| 13-2 | 13 | GGTATTCAAAGAACATACCTTTCAA | 66 | SEQ ID NO. 93 |
| 15-1 | 15 | ACAATAGAACATTCTTACCTCTGCC | 93 | SEQ ID NO. 94 |
| 16-1 | 16 | TCGTTATTTGGCAGCCAAAGTTACT | n/a | SEQ ID NO. 95 |
| 16-2 | 16 | GAGCCACAGCACAACCAAAGAAGCA | n/a | SEQ ID NO. 96 |
| 16-3 | 16 | TCCAAGGAGCCACAGCAC | n/a | SEQ ID NO. 97 |
| 16-4 | 16 | TTCCAAGGAGCCACAGCA | n/a | SEQ ID NO. 98 |
| 16-5 | 16 | TTCCAAGGAGCCACAGCACAACCAA | n/a | SEQ ID NO. 99 |
| 16-6 | 16 | AACAGAAATAAAACACAATCTACAC | n/a | SEQ ID NO. 100 |
| 16-7 | 16 | TTTCCAAGGAGCCACAGCACAACCA | 0 | SEQ ID NO. 101 |
| 16-8 | 16 | ACAATCTACACAATAGGACATGGAA | 56 | SEQ ID NO. 102 |
| 16-9 | 16 | CACAATCTACACAATAGGACATGGA | n/a | SEQ ID NO. 103 |
| 16-10 | 16 | ACACAATCTACACAATAGGACATGG | n/a | SEQ ID NO. 104 |
| 16-11 | 16 | GACTTTTTTTCTAACATCTTCACCT | n/a | SEQ ID NO. 105 |
| 16-12 | 16 | ATGGAACAACACACAGTTGATTTTT | n/a | SEQ ID NO. 106 |
| 16-13 | 16 | ATCGAACAAGACACAGTTGATTTTT | n/a | SEQ ID NO. 107 |
| 16-14 | 16 | GAGTGGAACAAGACACAGTTGATTT | n/a | SEQ ID NO. 108 |
| 16-15 | 16 | CACAATCTACACAATAAGACATGGA | n/a | SEQ ID NO. 109 |
| 20-1 | 20 | CAAGATGAGTGAAAATTGGACTCCT | 2 | SEQ ID NO. 110 |
| 20-2 | 20 | CGAAGGCACGAAGTGTCCATAGTCC | 29 | SEQ ID NO. 111 |
| 20-3 | 20 | AACAGAGTTTCAAAGTAAGGCTGCC | 8 | SEQ ID NO. 112 |
| 20-4 | 20 | AGTTGGCAGTATGTAAATTCAGAGC | 6 | SEQ ID NO. 113 |
| 20-5 | 20 | TTCTATTCTCATTTGGAACCAGCGC | 45 | SEQ ID NO. 114 |
| 20-6 | 20 | GGTAACAGCAATGAAGAAGATGACA | 35 | SEQ ID NO. 115 |
| 22-1 | 22 | ATGTCAATGAACTTAAAGACTCGGC | 59 | SEQ ID NO. 116 |
| 22-2 | 22 | GGCCAGATGTCATCTTTCTTCACGT | 65 | SEQ ID NO. 117 |
| 22-3 | 22 | ATCTTTGACAGTCATTTGGCCCCCT | 7 | SEQ ID NO. 118 |
| 22-4 | 22 | CCACCTTCTGTGTATTTTGCTGTGA | 45 | SEQ ID NO. 119 |
| 22-5 | 22 | TCTCTAATATGGCATTTCCACCTTC | 67 | SEQ ID NO. 120 |
| 22-6 | 22 | CCAGGACTTATTGAGAAGGAAATGT | 37 | SEQ ID NO. 121 |
| 22-7 | 22 | AAGCAGTGTTCAAATCTCACCCTCT | 63 | SEQ ID NO. 122 |
| 23-1 | 23 | ATCCAGTTCTTCCCAAGAGGCCCAC | 0 | SEQ ID NO. 123 |
| 23-2 | 23 | AGCTGATAACAAAGTACTCTTCCCT | 0 | SEQ ID NO. 124 |
| 23-3 | 23 | AAGTTATTGAATCCCAAGACACACC | 0 | SEQ ID NO. 125 |
| 23-4 | 23 | CTAAGTCCTTTTGCTCACCTGTGGT | 24 | SEQ ID NO. 126 |
| 24-1 | 24 | GATCACTCCACTGTTCATAGGGATC | 58 | SEQ ID NO. 127 |
| 24-2 | 24 | CTCATCTGCAACTTTCCATATTTCT | 53 | SEQ ID NO. 128 |
| 24-3 | 24 | ATTTCAGTTAGCAGCCTTACCTCAT | 66 | SEQ ID NO. 129 |

* percent of the mRNA transcripts that skip out the targeted exon

### Example 3: HCAI-CFTR Deletions in Fischer Rat Thyroid Cells

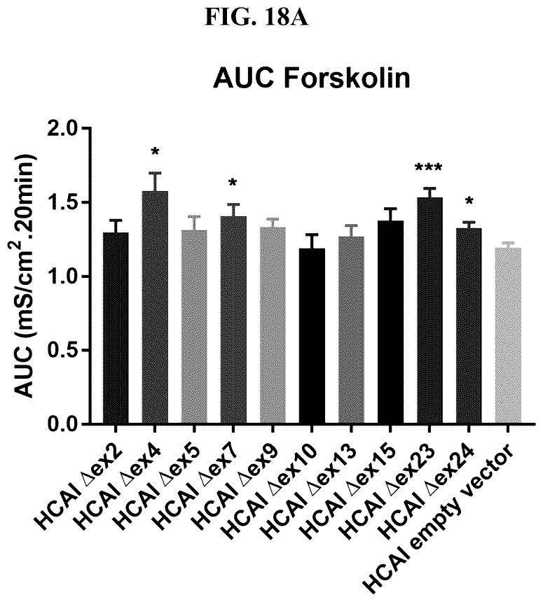

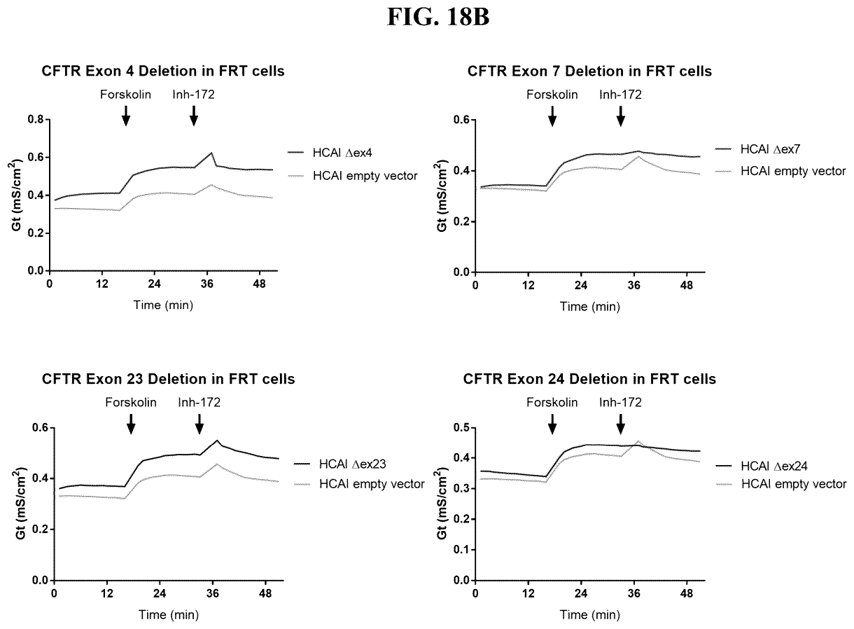

Fischer Rat Thyroid (FRT) cells, which lack functional CFTR, were stably transfected with nucleic acids encoding human CFTR with deletions of exon 2, 4, 5, 7, 9, 10, 13, 15, 23, or 24 (HCAIΔcx2. HCAIΔcx4, HCAIΔcx5, HCAIΔcx7. HCAIΔcx9. HCAIΔcx10. HCAIΔcx13. HCAIΔcx 15 HCAIΔcx23, or HCAIΔcx24). FRT cells stably the HCAI-CFTR exon deletions were seeded onto HTS Transwell®-24 well permeable filter plates (0.4 μM pore size, Polyester, Corning) and differentiated for 2 weeks. Transepithelial conductance was assessed in Gt assays that were performed 14 days after cell seeding. The data were recorded with 24-channel transepithelial current clamp (TECC)_Robot system (Design, Belgium). HCAI-CFTR activity was measured by the change in Gt upon stimulation with forskolin (10 μM). CFTRInh-172 (10 μM) was used to confirm CFTR dependence. A comparison of the AUC forskolin-stimulated HCAI-CFTR exon deletion channel activity to HCAI empty vector is shown in FIG. 18A (error bars represent SEM; * p<. 05, *** p<. 001, n=4, two-tailed t-test compared to HCAI empty vector). Representative Gt traces of CFTR exon 4, exon 7, exon 23, and exon 24 deletion constructs in comparison to HCAI empty vector are shown in FIG. 18B.

### Example 4: Antisense Oligonucleotides Induce Exon Skipping of Exons with Nonsense Mutations in CFTR In Vivo and Restore the CFTR Reading Frame

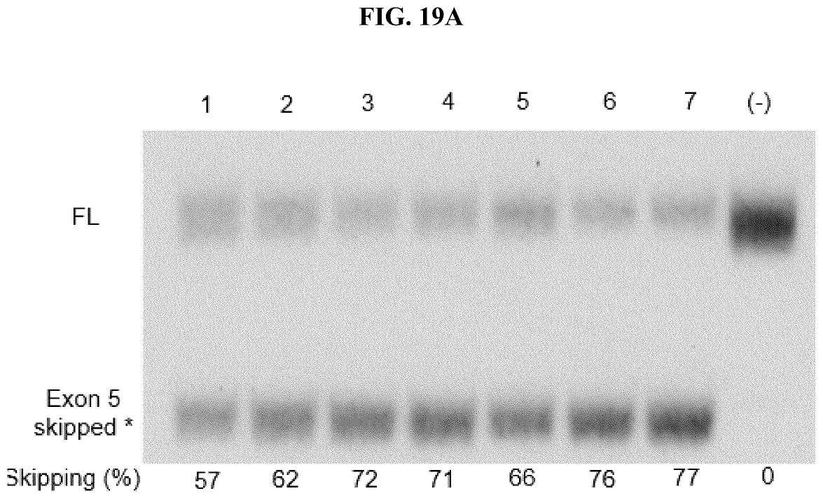

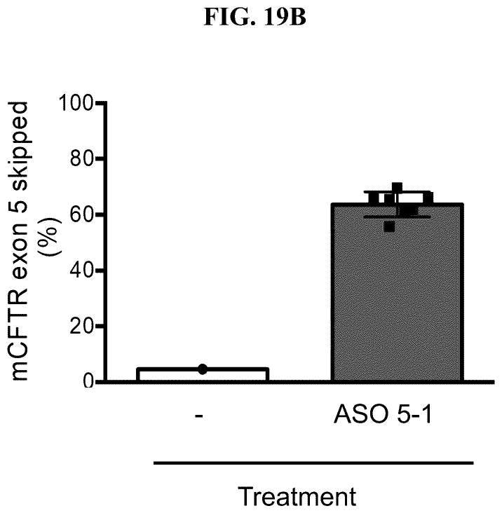

ASO 5-1 (SEQ ID NO:12) was tested in mice and shown to induces CFTR exon 5 skipping. Intracerebroventricular (ICV) injection of mCFex5-1 was performed in wild-type mice (C57BI/6) on post-natal day 2, and mice were euthanized on post-natal day 12. RNA was collected from the hippocampus. Radioactive RT-PCR of CFTR RNA isolated from hippocampus is shown in FIG. 19A (splice isoforms are labeled and exon 5 skipping quantification is shown at the bottom). A quantitation of the RT-PCR analysis of the RNA exon 5 skipping induced by ASO 5-1 treatment is shown in FIG. 19B.

### Example 5: Antisense Oligonucleotides to Correct CFTR 2789+5 G>a Splicing Mutation

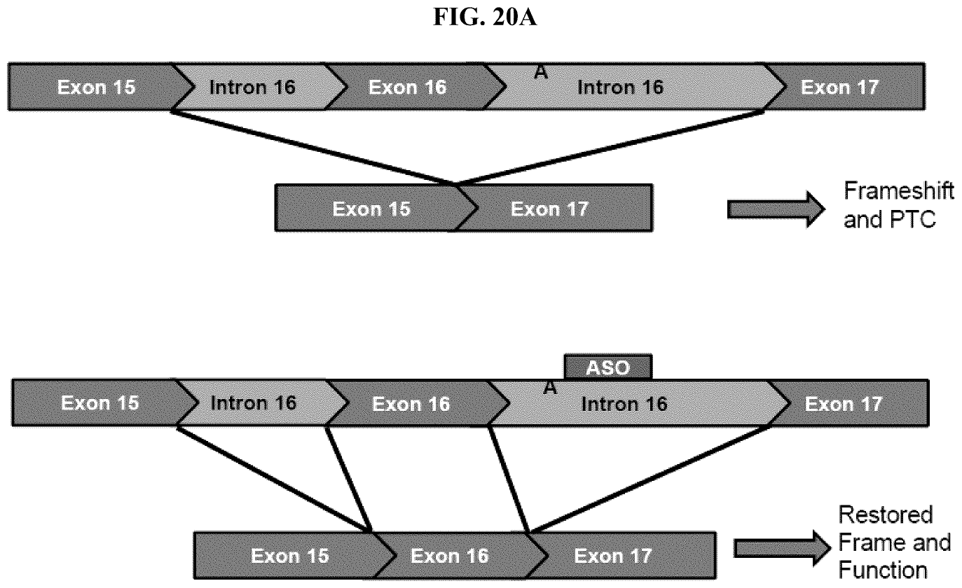

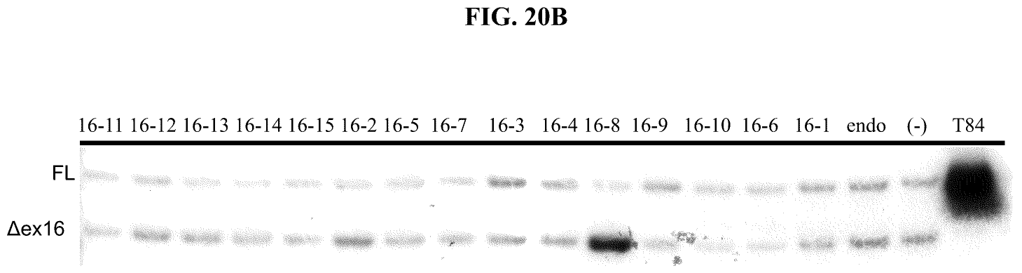

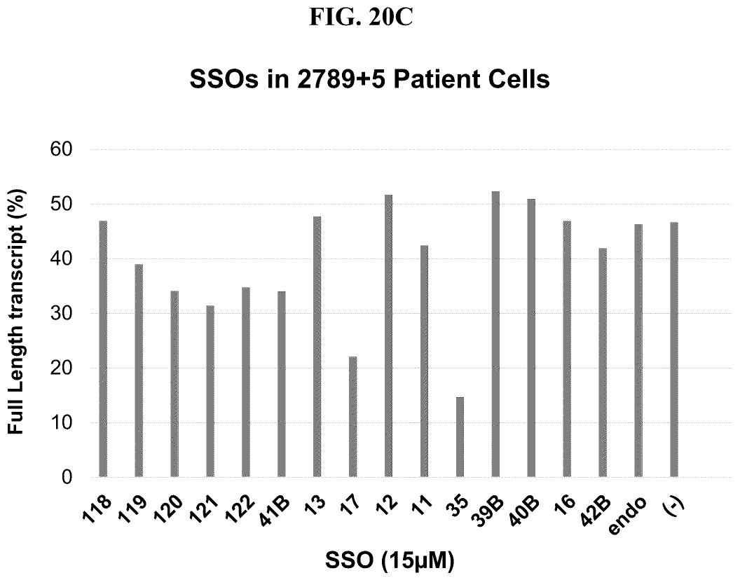

Antisense oligonucleotides were designed that increase correct splicing in 2789+5 G>A in patient lymphoblast cells lines. The lymphoblast cell line 11859, which is homozygous for the 2789+5 G>A mutation, was transfected with ASOs that were designed to correct the splicing in CFTR 2789+5 G>A (ASO concentration of 15μM; and cells were treated for 48 hours). Correction of CFTR splicing in 2789+5 the lymphoblasts using ASOs is shown in FIG. 20B (CFTR spliced isoforms are labeled; T84 cells were analyzed as a positive control for wild-type CFTR splicing). A quantitation of the RT-PCR analysis of the RNA splice correction induced by ASO treatment in patient lymphoblast cells is shown in FIG. 20C. A summary of the 2789+5 ASOs targets, sequences, and correction activity in patient lymphoblast cells is shown in Table 3.

TABLE 3
ASO sequences tested in the 2789 + 5
lymphoblast cell line.

| | Target | Sequence (5′ to 3′) | % Full |
|---|---|---|---|
| Name | Region | (SEQ ID NO.) | Length |
| 16-11 | Intron 15 | GACTTTTTTTCTAACATCTTCACCT | 47 |
| | | (SEQ ID NO.: 105) | |
| 16-12 | Intron 15 | ATGGAACAACACACAGTTGATTTTT | 39 |
| | | (SEQ ID NO.: 106) | |
| 16-13 | Intron 15 | ATCGAACAAGACACAGTTGATTTTT | 34 |
| | | (SEQ ID NO.: 107) | |
| 16-14 | Intron 15 | GAGTGGAACAAGACACAGTTGATTT | 31 |
| | | (SEQ ID NO.: 108) | |
| 16-9 | Exon 16 | CACAATCTACACAATAAGACATGGA | 35 |
| | | (SEQ ID NO.: 109) | |
| 16-2 | Exon 16 | GAGCCACAGCACAACCAAAGAAGCA | 34 |
| | | (SEQ ID NO.: 96) | |
| 16-5 | Exon 16 | TTCCAAGGAGCCACAGCACAACCAA | 48 |
| | | (SEQ ID NO.: 99) | |
| 16-7 | Exon 16 | TTTCCAAGGAGCCACAGCACAACCA | 22 |
| | | (SEQ ID NO.: 101) | |
| 16-3 | Exon 16 | TCCAAGGAGCCACAGCAC | 52 |
| | | (SEQ ID NO.: 97) | |
| 16-4 | Exon 16 | TTCCAAGGAGCCACAGCA | 42 |
| | | (SEQ ID NO.: 98) | |
| 16-8 | Intron 16 | ACAATCTACACAATAGGACATGGAA | 15 |
| | | (SEQ ID NO.: 102) | |
| 16-9 | Intron 16 | CACAATCTACACAATAGGACATGGA | 52 |
| | | (SEQ ID NO.: 103) | |
| 16-10 | Intron 16 | ACACAATCTACACAATAGGACATGG | 51 |
| | | (SEQ ID NO.: 104) | |
| 16-6 | Intron 16 | AACAGAAATAAAACACAATCTACAC | 47 |
| | | (SEQ ID NO.: 100) | |
| 16-1 | Intron16 | TCGTTATTTGGCAGCCAAAGTTACT | 42 |
| | | (SEQ ID NO.: 95) | |

### Example 6: Antisense Oligonucleotides to Correct CFTR 3272-26 A>G Splicing Mutation

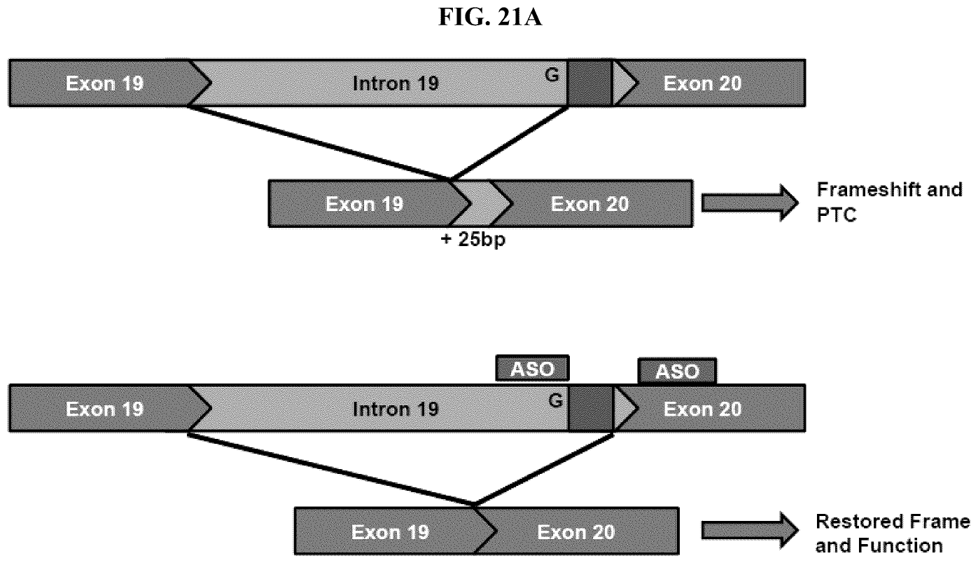

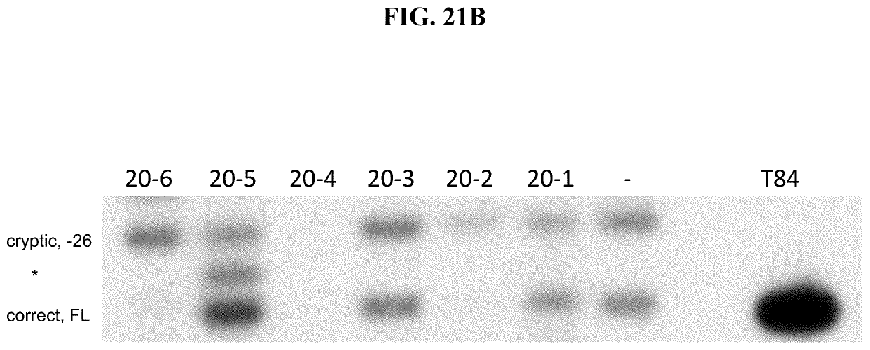

Antisense oligonucleotides were designed that increase correct splicing in 3272-26 A>G mutation in patient lymphoblast cell lines. The lymphoblast cell line 18801 (18801 is from a male donor with one allele carrying the 3272-26 A>G mutation, and no mutation was identified in the second allele) was transfected with ASOs that were designed to correct splicing in CFTR 3272-26 A>G (ASOs were transfected with Endo-Porter, the ASO concentration was 15 μM, and cells were treated for 48 hours). Correction of CFTR splicing in CFTR 3272-26 A>G in the lymphoblast cells using ASOs is shown in FIG. 21B (CFTR spliced isoforms are labeled; T84 cells were analyzed as a positive control for wild-type CFTR splicing). A summary of the CFTR 3272-26 A>G ASOs targets, sequences, and correction activity in patient lymphoblast cells is shown in Table 4.

TABLE 4

| | Target | Sequence (5′ to 3′) | % Full- |
|---|---|---|---|
| Name | Exon | (SEQ ID NO.) | Length |
| 20-1 | 20 | CAAGATGAGTGAAAATTGGACTCCT | 60 |
| | | (SEQ ID NO.: 110) | |
| 20-2 | 20 | CGAAGGCACGAAGTGTCCATAGTCC | 3 |
| | | (SEQ ID NO.: 111) | |
| 20-3 | 20 | AACAGAGTTTCAAAGTAAGGCTGCC | 50 |
| | | (SEQ ID NO.: 112) | |
| 20-4 | 20 | AGTTGGCAGTATGTAAATTCAGAGC | nd |
| | | (SEQ ID NO.: 113) | |
| 20-5 | 20 | TTCTATTCTCATTTGGAACCAGCGC | 56 |
| | | (SEQ ID NO.: 114) | |
| 20-6 | 20 | GGTAACAGCAATGAAGAAGATGACA | 12 |
| | | (SEQ ID NO.: 115) | |

### Example 7: Antisense Oligonucleotides to Correct CFTR 3849+10 kb C>T Splicing Mutation

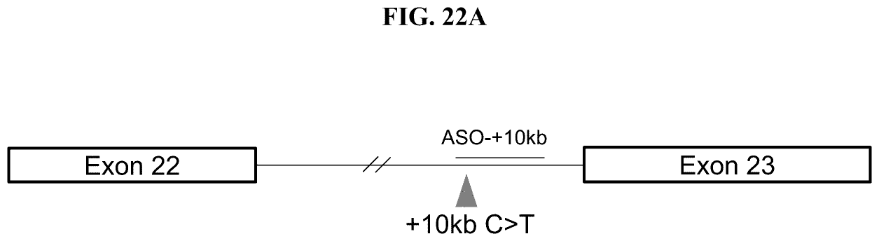

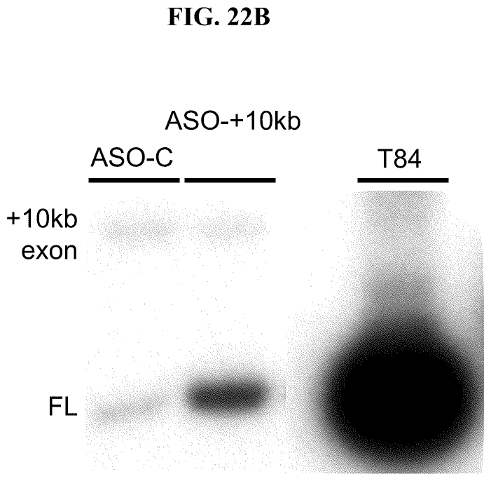

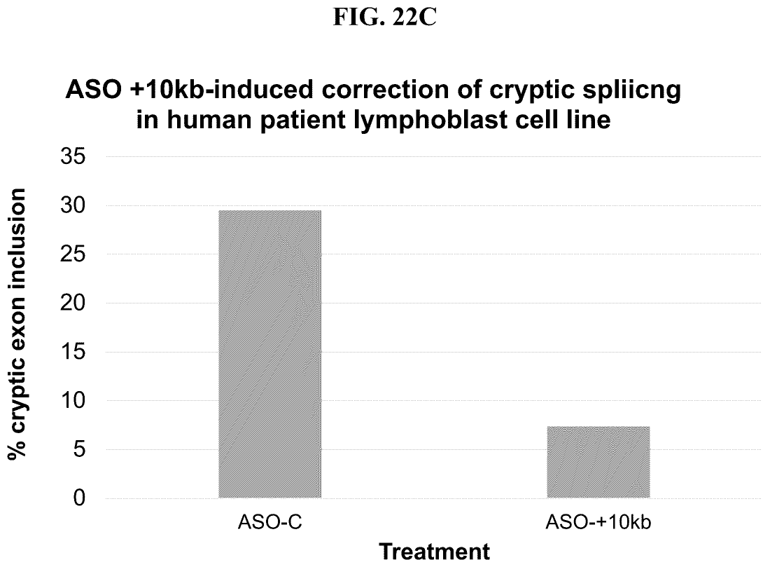

Antisense oligonucleotides were designed to repair the 3849+10 kb C>T splice mutation and restore CFTR function. The C>T mutation creates a cryptic 5′ splice site that results in the inclusion of an 84 bp insert from intron 22, and the mutated allele produces both wild-type and aberrantly spliced transcripts. The lymphoblast cell line 18860 (18860 is homozygous for 3849+10 kb CFTR mutation) was transfected with ASOs that were designed to correct splicing in 3849+10 kb C>T (ASOs were transfected with Endo-Porter, the ASO concentration was 15 μM, and cells were treated for 48 hours). Correction of CFTR splicing in 3849+10 kb C>T in the lymphoblast cells using ASOs is shown in FIGS. 22B and 22C (CFTR spliced isoforms are labeled; T84 cells were analyzed as a positive control for wild-type CFTR splicing). A summary of the CFTR 3849+10 kb C>T ASO target, sequence, and correction activity in patient lymphoblast cells is shown in Table 5.

TABLE 5

| | Target | Sequence (5′ to 3′) | % Full- |
|---|---|---|---|
| Name | Exon | (SEQ ID NO.) | Length |
| ASO-+10kb | Intron | CCTTTCAGGGTGTCTTACTCAC | 93 |
| | 22 | CAT (SEQ ID NO.: 150) | |

### Example 8. Analyzing CFTR Function in Patient Epithelial Cells Treated with ASOs

Primary patient human bronchial epithelial (HBE) cells (cells are compound heterozygotes with the 3849+10kbC>T and ΔF508 mutation) were seeded on HTS Transwell®-24 well permeable filter plates (0.4 μM pore size, Polyester, Corning) and switched to air/liquid interphase after 3 days. Ieq measurements were carried out 99 days after seeding. Cells were treated basolaterally with C18 (Corr951/VX-661, 6 μM) or DMSO (0.1%), and apically transfected with ASO-+10 kb (SEQ ID NO:150 at 20 μM or 80 μM) or ASO-C(20 μM or 80 μM: 5′ CCTCTTACCTCAGTTACAATTTATA 3′-SEQ ID NO:151) 4 days before Ieq measurements were taken. C18 is a corrector compound that improves F508del-CFTR folding and function. Cells were transfected using EGTA (4 mM) and Endo-Porter (GeneTools) for 10 hours, then EGTA was taken off and the cells were transfected again using Endo-Porter in the absence of EGTA. The data were recorded with 24-channel transepithelial current clamp (TECC) Robot system (Design, Belgium). Sodium current was inhibited by benzamil (6 μM) and CFTR activity was measured by the change in Ieq upon stimulation with forskolin (10 μM) and VX-770/KALYDECO™/Ivacaftor (1 μM), which is a CFTR potentiator that improves the transport of chloride through the CFTR channel. Inhibition with bumetanide/BUMEX™/BURINEX™ (20 μM) was used to confirm CFTR dependence.

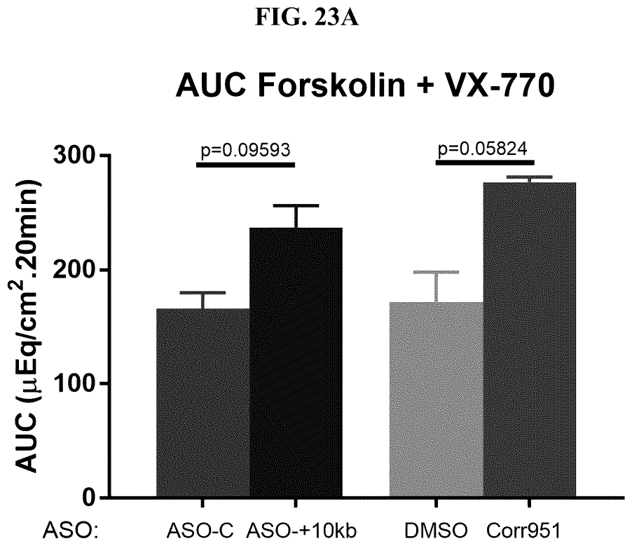

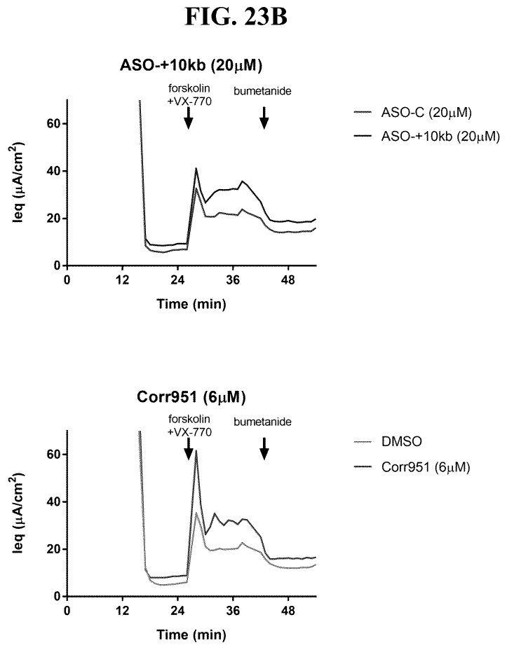

The results demonstrate that ASO-+10 kb (SEQ ID NO: 150) rescues CFTR function similar to Con951/VX-661 (CFTR corrector 106951 (1-(benzo[d][1.3]dioxol-5-yl)-N-(5-((S)-(2-chlorophenyl) ((R)-3-hydroxypyrrolidin-1-yl)methyl) thiazol-2-yl) cyclopropanecarboxamide)) in patient HBE cells. As shown in FIG. 23. ASO-+10 kb rescues CFTR function similar to Corr951 in patient HBE cells. FIG. 23A is a graph showing the area under the curve (AUC) of time from forskolin+VX-770-stimulation of CFTR channels following indicated treatment (error bars represent SEM; two-tailed t-test, n=2). FIG. 23B depicts representative Ieq traces of treatment (Corr951 or ASO-+10 kb) compared to control (ASO-C, top, or DMSO, bottom).

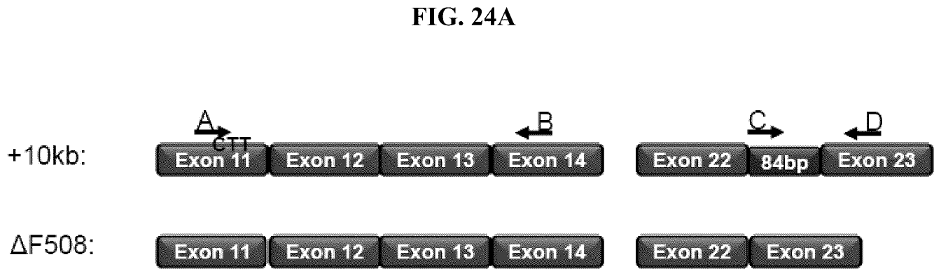

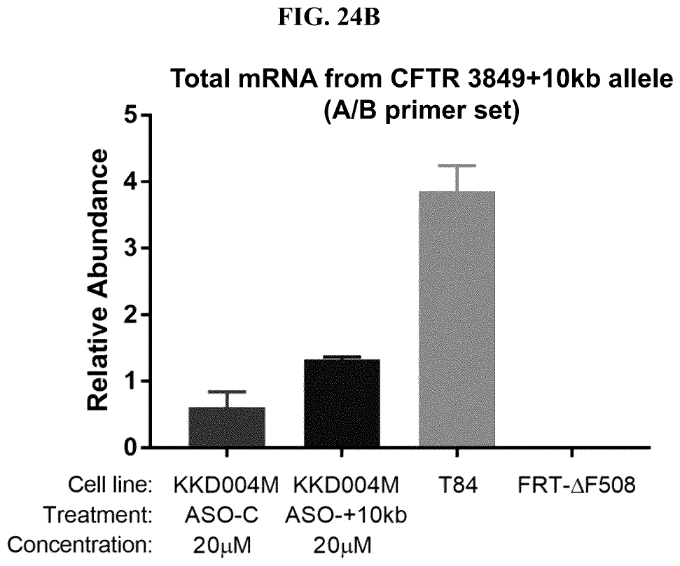

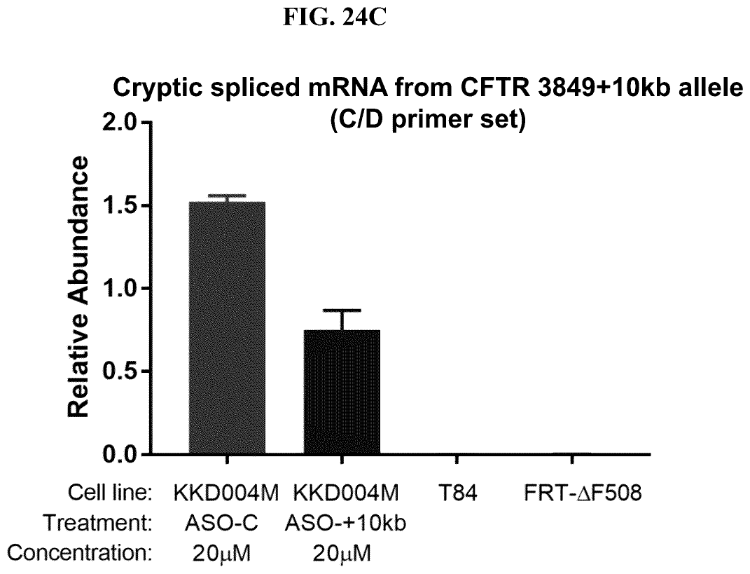

Additionally, the results show that ASO-+10 kb (SEQ ID NO: 150) increases WT splicing in 3849+10 kb patient HBE cells. Primary patient HBE cells are heterozygous for the 3849+10kbC>T mutation were transfected with ASO-+10 kb (20 μM). Total mRNA was isolated, reverse transcribed, and analyzed for splice correction using SYBER™ Green quantitative PCR. FIG. 24A depicts the primer sets used to analyze splice correction by ASO-10+kb (primer set A-B is designed to amplify ASO corrected WT isoform splicing specific to the splice mutant allele, and primer set C-D is designed to analyze the amount of uncorrected mutant splicing). FIG. 24B shows a quantification of total mRNA transcribed from the CFTR 3849+10 KB allele, and indicates an increase with ASO-+10 kb treatment (A-B primer set). FIG. 24C shows a quantification of mutant, cryptically spliced mRNA isoform, and shows decrease of aberrant mRNA with ASO-+10 kb treatment (C-D primer set).

### Example 9. Open Reading Frame Correction Using Antisense Oligonucleotides for the Treatment of Cystic Fibrosis

CFTR gene mutations that introduce premature termination codons account for ˜10% of cystic fibrosis cases. This mutation type is associated with a severe form of the disease, typically a consequence of low CFTR mRNA levels resulting from degradation by nonsense mediated mRNA decay (NMD), and production of a truncated, non-functional CFTR protein. Current therapeutics for CF are less effective in patients with these types of mutations, likely due to the instability of the mRNA and lack of the natural C-terminal portion of the protein. Antisense technology is one promising therapeutic strategy for these types of mutations that has not been widely explored for the disease. Splice-switching antisense oligonucleotides (ASOs) can be designed to modify gene expression by directly modulating pre-mRNA splicing. ASO-mediated skipping of exons to restore the open reading frame of RNA with frameshift mutations or premature termination codons (PTC) has been successfully applied to treat a number of disorders. This Example shows that CFTR lacking the amino acids encoding exon 23 retains the ability to conduct chloride. The functionality of this CFTR isoform is enhanced by corrector and modulator drugs currently in use clinically to treat certain CFTR mutations. ASO-induced exon 23 skipping in an immortalized bronchial epithelial cell line expressing CFTR W1282X results in a dose-dependent increase in CFTR mRNA and a corresponding recovery of chloride channel activity. These results support the use of ASOs in treating CF patients with rare CFTR class I mutations in exon 23 that result in unstable CFTR mRNA and truncations of the CFTR protein.

To date, approximately 2,000 CF-associated variants in CFTR have been identified with variable prevalence and disease severity. Current therapeutics approved and in development focus on specific patient subpopulations with the most common mutations in classes II and III. Currently, there are only four FDA approved drugs that directly target CFTR dysfunction. Of these, ivacaftor potentiates function of CFTR by increasing the probability of channel opening to increase anion ion conductance of CFTR gating variants, lumacaftor and tezacaftor act as chemical chaperones to correct processing and trafficking of CFTR to the cell surface, and a new corrector, elexacaftor, works in combination with tezacaftor and ivacaftor. There is a critical need for therapies for patients with rare CFTR mutations, in particular nonsense variants that create a premature termination codon and thereby a truncated protein.

Gene mutations that result in PTCs are challenging to treat because they not only encode a truncated protein product, by virtue of the early termination, but the mRNA intermediate is often a target of nonsense-mediated mRNA decay (NMD), a cellular quality-control mechanism whereby mRNA with PTCs are degraded. Thus, PTCs result in lower protein expression and the production of a truncated protein from the limited amount of translated variant mRNA. Current approaches in the development of treatments for PTCs involve screening for molecules that stabilize the mRNA transcripts, that is block NMD, and also increase translational read-through of PTCs to recover full-length protein expression. Another approach to treating disease associated with PTCs is to eliminate the PTC by inducing skipping of the exon encoding the variant. This approach requires skipping of the exon to retain the proper reading frame. This so-called reading frame correction has shown promise as a therapeutic approach in the FDA-approved splice-switching antisense oligonucleotides (ASOs) targeting PTCs in Duchenne's Muscular Dystrophy.

The second most common CF-associated nonsense mutation is W1282X, located in exon 23. This nonsense mutation truncates CFTR at amino acid 1281 (CFTR1281), removing ˜60% of the nucleotide binding domain 2 (NBD2) but retaining most of the full-length protein (1281 vs 1480 amino acids). The truncated protein may have processing and/or gating defects as an increase in channel function can be achieved in W1282X-CFTR cells by potentiator and corrector treatment. Because of the premature termination codon created by the mutation, W1282X-CFTR mRNA is subjected to NMD, leading to a decrease in mRNA and consequently protein abundance and thereby limiting the effectiveness of protein modulator drugs. Small molecule compounds that inhibit NMD have been shown to increase W1282X-CFTR expression but to date no effective drug candidates targeting NMD have been clinically approved.

Another approach to stabilizing W1282X-CFTR is to eliminate the PTC by using an ASO that induces the exclusion of exon 23 during the process of pre-mRNA splicing. Such skipping of exon 23 results in the production of a CFTR mRNA with an intact open-reading frame with a deletion of the 52 amino acids encoded by the exon. This strategy may have a therapeutic benefit not only by producing a potentially more functional protein isoform with a restored C-terminus, as suggested by biochemical and functional studies of CFTR, but also by stabilizing and increasing protein expression from the allele, and thereby improving efficacy of current FDA-approved CF therapeutics as well as other potentiators that are effective for mutations in NBD2.

ASOs are a promising therapy in personalized treatment to modulate pre-mRNA splicing to induce skipping of target exons. ASOs are short, oligonucleotides modified in their sugar and back-bone structure to be stable and specific, with long lasting effects on splice modulation over time. Recent FDA approval of ASOs for a number of diseases exemplifies their therapeutic potential. Specific to the CF field, aerosolized delivery of antisense oligonucleotides has shown to be effective making ASO treatment delivered directly to the airways of CF patients a promising approach. Unlike other antisense therapies in development for CF that target other ion channels involved in the epithelial fluid secretory process, the ASO approach disclosed herein will provide a therapy for class I mutations by targeting the underlying defect in the CFTR gene. Despite recent advances in CFTR drug development, where currently ˜90% of CF patients are eligible for CF drug therapy, ASO treatment provides a potential therapeutic for CF patients with rare stop mutations that results in severe forms of the disease, further closing this therapeutic gap for CF patients left behind.

This Example demonstrates that skipping of exon 23 rescues CFTR mRNA expression disrupted by the PTC and produce a CFTR isoform with partial function that is responsive to CFTR modulators. As shown herein, expressing CFTR-Δ23, with deletion of exon 23, in Fischer rat thyroid (FRT) cells, which lack endogenous CFTR, has residual CFTR function as assessed by forskolin-induced conductance. This activity is further stimulated by treatment with recently FDA-approved CFTR modulators. Furthermore, ASO-mediated exon 23 skipping of W1282X-CFTR RNA in an immortalized human W1282X-CFTR bronchial epithelial cells line increases overall CFTR mRNA levels and recovers channel activity as measured by forskolin-induced conductance. The combination of ASO treatment with CFTR modulator treatment, which is the current standard of care for these patients, results in CFTR activity that is greater than either treatment alone. Together, these results suggest that ASO-induced exon 23 skipping in CF cases caused by nonsense mutations in the exon, is a promising therapeutic adjuvant.

### Materials and Methods

Generation of exon deletion constructs: CFTR-Δex23 was created from the synthetic CFTR high codon adaption index (HCAI) construct subcloned in the pcDNA3.1/Neo (+) vector (Shah et al. 2015) using the Q5 Site-Directed Mutagenesis Kit (NEB) with primers flanking exon 23 (Table 6). The plasmid was sequenced to confirm complete exon deletion and an intact reading frame and then stably transfected into Fischer Rat Thyroid (FRT) cells using lipofectamine LTX (Thermo Fisher) and OptiMEM (Thermo Fisher) on 6-well plates for 48 hours. Cells were transferred to T75 flasks and clonal cell lines were selected with G418 (300 μg/ml) for one week. After selection, cells were maintained in media supplemented with G418 (150 μg/ml).

Cells and culture conditions: FRT cell lines were cultured in F12 Coon's modification media (Sigma. F6636) supplemented with 10% FBS and 1% Penicillin-Streptomycin (PenStrep). 16hBEge-W1282X-CFTR cell lines were obtained from the Cystic Fibrosis Foundation (CFF) and cultured according to their instructions in MEM media (Gottschalk et al. 2016; Valley et al. 2019). Single-cell clones of the 16hBEge-W1282X-CFTR cell lines were isolated and selected for high resistance. Two clonal cell lines. 3F2 and 9A6, were used for functional analysis. For functional analysis. FRT cells and 16hBE-W1282X-CFTR clones were plated on Costar 24-well high-throughput screening filter plates (0.4 μM pore size, Polyester, Corning, catalog #CLS3397) and grown in a liquid/liquid interface (180 μl apical/700 μl basolateral) in a 37° C. incubator with 90% humidity and 5% CO₂ for 1 week. Media was replaced 3 times a week.

Antisense oligonucleotides: Splice-switching antisense oligonucleotides are 25-mer phosphorodiamidate morpholino oligomers (Gene-Tools, LLC) (Table 6). ASO-23-4 (SEQ ID NO: 126) induces skipping of exon 23. ASO-23-3 (SEQ ID NO: 125) blocks a cryptic 5′ splice site partially activated by ASO-23-4. A non-targeting ASO was used as a negative control. ASO-C. (Gene Tools, standard control oligo). ASOs were formulated in sterile water.

ASO cell transfection: The 16HBEge-W1282X-CFTR clonal cell line was transfected on filter plates 4 days post plating. Cells were transfected with ASOs apically in 100 μl of complete MEM media with Endo-Porter (6 μl/ml; Gene-Tools) at indicated concentrations for 48 hours (Summerton 2005). After transfection, the media was removed and replaced with media containing VX-661 (3 μM) and VX-445 (1 μM) or DMSO (0.2%) for 24 hours until functional analysis. For the functional analysis the media was removed and replaced with HEPES-buffered Coon's F12 media.

RNA isolation and RT-PCR: RNA was extracted from cells using TRIzol according to manufacturer instructions (Thermo Fisher Scientific). Reverse transcription was performed on total RNA using the GoScript Reverse Transcription System with an oligo-dT primer (Promega). Splicing was analyzed by radiolabeled PCR of resulting cDNA using GoTaq Green (Promega) supplemented with α-³²P-deoxycytidine triphosphate (dCTP). Primers for amplification are reported in Table 6 and include primer sets flanking the deleted HCAI-CFTR exon 23. (hCFTRx22F. hCFTRex24R) and primers for human-actin analyzed as a control (hβ-actinFor, hβ-actinRev). Reaction products were run on a 6% non-denaturing polyacrylamide gel and quantified using a Typhoon 7000 phosphorimager (GE Healthcare) or ImageJ software.

Real-time qPCR: Real-time qPCR was performed with Prime Time Gene Expression Master Mix and PrimeTime qPCR probe assay kits human non-F508del-CFTR (IDT, hCFTR-F508) transcripts normalized to human β-actin (IDT. HsPT.39a.2214847) (Table 9). All reactions were analyzed in triplicate on 96-well plates and averaged together to comprise one measurement. Real-time PCR was performed on an Applied Biosystems (ABI) ViiA 7 Real-Time PCR System with the thermal-cycling protocol: stage 1-50° C. for 2 min. 95° C. for 3 min; stage 2-40 cycles of 95° C. for 15 s. 60° C. for Imin to ensure an amplification plateau was reached. Results were analyzed by the ΔΔCT method (Livak and Schmittgen 2001).

Protein isolation and automated immunoblot (western) analysis: Cell lysates for immunoblot analysis were prepared for functional analysis using NP-40 lysis buffer (1% Igepal. 150 mM NaCl. 50 mM Tris-HCl pH7.6) supplemented with 1x protease inhibitor cocktail (Sigma-Aldrich, cat #11836170001). Protein concentration was measured using a Coomassie (Bradford) protein assay (Thermo Fisher, cat #23200). Cell lysates were prepared and diluted to 1 mg/ml using the sample preparation kit (Protein Simple) for an automated capillary western blot system. WES System (Protein Simple) (Harris 2015; Kannan et al. 2018). Cell lysates were mixed with 0.1x sample buffer and 5x fluorescent master mix for a final protein lysate concentration of 0.2 mg/ml. Samples were incubated at room temperature for 20 minutes and then combined with biotinylated protein size markers, primary anti-CFTR antibodies 432 (Riordan lab UNC. Cystic Fibrosis Foundation, diluted 1:100 with milk-free antibody diluent), anti-β-actin (C4. Santa Cruz Biotechnology, diluted 1:50 with milk-free antibody diluent), horseradish peroxidase (HRP)-conjugated secondary antibodies, chemiluminescence substrate and wash buffer and dispensed into respective wells of the assay plate and placed in WES apparatus. Samples were run in triplicate. Signal intensity (area) of the protein was normalized to the peak area of the loading control C4. β-actin. Quantitative analysis of the CFTR B and C-bands was performed using Compass software (Protein Simple).

Automated conductance assay: Stably transfected FRT cells were treated with the corrector C18 (6 μM) (VRT-534. VX-809/lumacaftor analog). VX-445+VX-661 (3 μM+1 μM final concentration) or vehicle (0.1% or 0.2% DMSO) at 37° C. 16hBEge-W1282X-CFTR clones were treated similarly with VX-445+VX-661 (3 μM+1 μM) or vehicle (0.2% DMSO) (Eckford et al. 2014). Twenty-four hours later the cells were switched from growth media to HEPES-buffered (pH 7.4) F12 Coon's modification media (Sigma, F6636) apically and basolaterally and allowed to equilibrate for one hour at 37° C. without CO₂. To obtain the conductance measurements, the transepithelial resistance was recorded at 37° C. with a 24-channel TECC robotic system (EP Design, Belgium) as previously described (Vu et al. 2017). Briefly, for the FRT cells, baseline measurements were taken for ˜20 minutes. Forskolin (10 μM) was added to the apical and basolateral sides and measurements were recorded for 20 minutes. The cells were then treated apically and basolaterally with potentiator. VX-770 (1 μM), and measurements were taken for an additional 20 minutes. The cells were then treated with an inhibitor of CFTR, Inh-172 (20 μM), on the apical and basolateral sides for 30 minutes. The 16hBEge-W1282X-CFTR clone was measured similarly with the exception that forskolin and VX-770 were added to the cells at the same time. Measurements were taken at two-minute intervals (10 measurements/addition). Gt was calculated by the reciprocal of the recorded Rt (Gt=1/Rt), after Rt was corrected for solution resistance (Rs), and plotted as conductance traces. To estimate average functional response trajectories during each test period, area under the curve measurements of forskolin and forskolin+VX-770 were calculated using a one-third trapezoidal rule for the entirety of each test period using Excel. The average of two identically treated wells was calculated for each plate to obtain one biological replicate used in the final mean±SEM graphed for each exon deletion.

Statistics: Statistical analyses were performed using GraphPad PRISM 8.2.1 or Microsoft Excel. A two-tailed one-sample t-test was used to assess significant changes of one test-group normalized to a control. One-way ANOVA analysis with a post-hoc test (Tukey's) was used to assess significant differences when comparing more than two groups. Two-way ANOVA analysis was used when comparing two independent variables with a post-hoc test (Tukey's) to assess significant differences between and within groups. When assessing significance differences within groups of paired data Sidak's post-hoc test was used. The specific statistical test used in each experiment can be found in the associated figure legend.

### Results

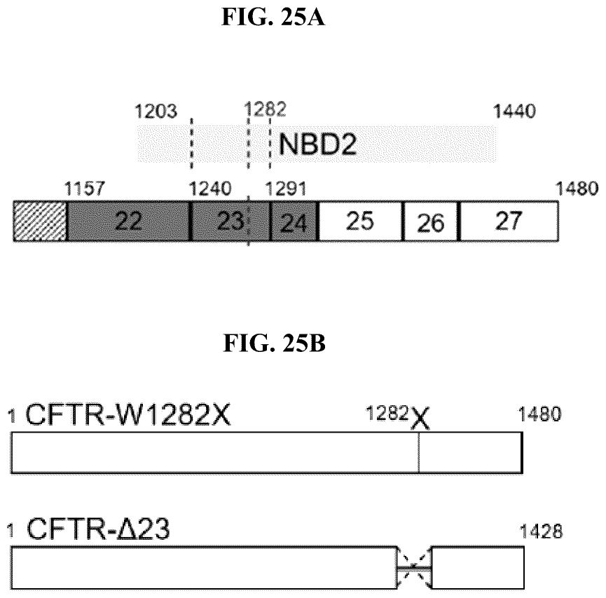

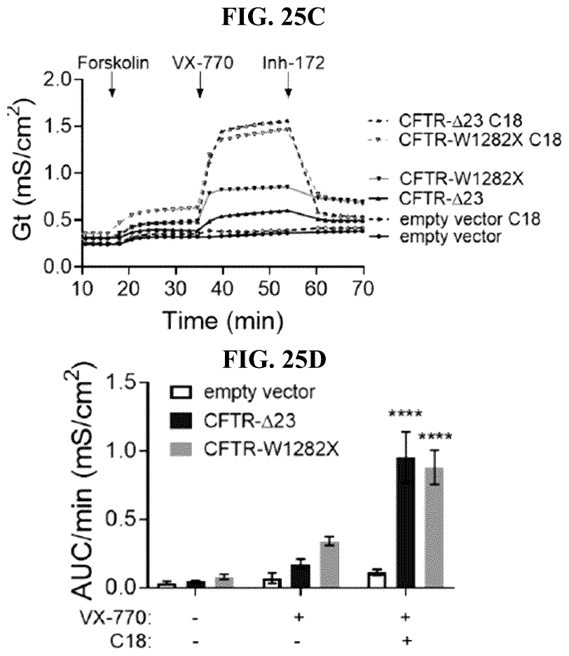

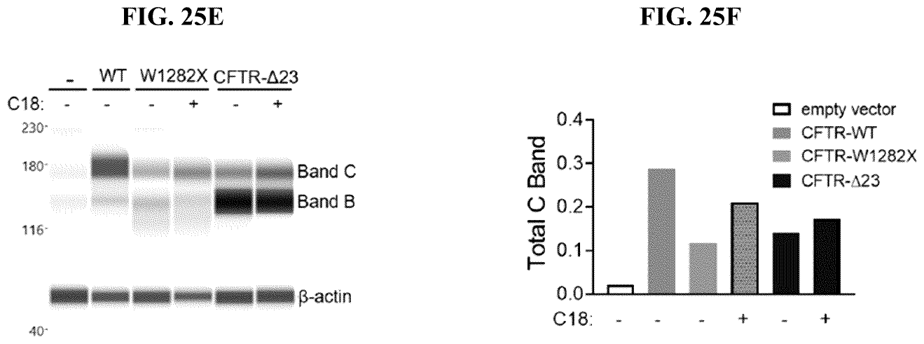

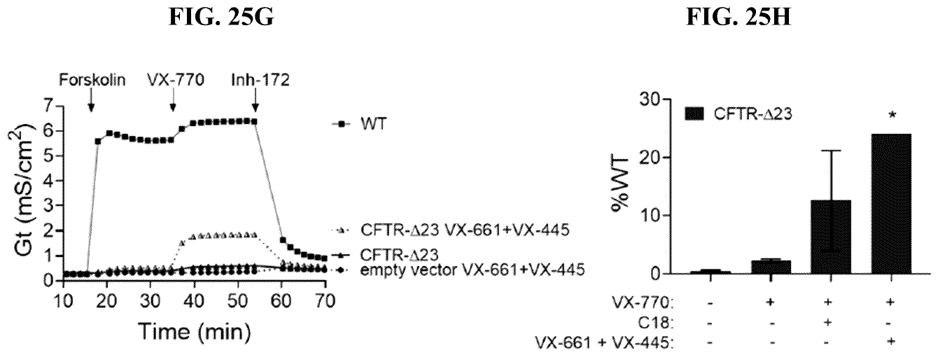

Deletion of CFTR exon 23 results in a partially functional CFTR isoform that is responsive to current CF modulators: To test the function of CFTR protein lacking amino acids encoded by exon 23, a CFTR cDNA deletion construct (CFTR-Δex23) was created, and CFTR expression and function was analyzed after stable transfection in FRT cells. A CFTR cDNA plasmid designed for high expression using a high codon adaption index for codon selection (HCAI) was used to improve exogenous CFTR expression. Exon 23 deletion in the mRNA was confirmed via RT-PCR (FIG. 25A, 25B).

To analyze retained CFTR function, transfected FRT cells were plated on transwell filter plates and grown 7 days to confluent monolayers. Semi-functional CFTR isoforms are expected to be responsive to modulator treatment and because current CF therapeutics include combination treatment with CF modulators, the cells were treated with C18, an analog of the corrector VX-809/lumacaftor, for 24 hours, and the potentiator VX-770 after forskolin addition to test if CFTR-Δex23 activity could be further improved. Transepithelial resistance measurements were recorded from the monolayers to calculate transepithelial conductance (Gt=1/Rt) responses attributed to forskolin-stimulated activation of CFTR and inhibition by Inh-172, a specific inhibitor of CFTR. Measurements were plotted as conductance traces (FIG. 25C). CFTRΔex23 did not have significant conductance without modulator treatment but had a significant (p<. 0.0001) increase in forskolin-induced conductance compared to cells transfected with the empty vector (white bars) as measured by the area under the curve per minute of the 20 minute test periods, with C18+VX-770 treatment (FIG. 25D) (AUC/min: empty vector=0.032+0.015 mS/cm². CFTR-Δ23=0.592=0.181 mS/cm²).

Previous studies have reported that stable expression of CFTR1281 results in a semi-functional CFTR protein that can be corrected by CF modulator treatment and that VX-770 treatment is effective for some patients homozygous with the W1282X mutation (Haggie et al. 2017; Mutyam ct al. 2017). To compare CFTR-Δ23 activity with truncated CFTR W1282X activity. FRT cells were transfected with a plasmid expressing CFTR W1282X and CFTR expression and function was analyzed (FIG. 25). CFTR-W1282X retained some CFTR function that was further improved with C18 and VX-770 treatment. CFTR-W1282X activity was similar to that of CFTR-Δ23 expression, which also provided significant (p<0.0001) conductance with combined modulator treatments, when compared to the empty vector (FIG. 25C, 25D) (AUC/min: empty vector=0.111+0.022 mS/cm². CFTR-Δ23=0.951+0.187 mS/cm². CFTR-W1282X=0.880+0.127 mS/cm²). Treatment with the corrector C18 also had a similar effect with both W1282X and Δex23 expression and also increased the abundance of the mature CFTR band C (FIG. 25D). Immunoblot analysis of CFTR protein isolated from cells after functional analysis verified expression of CFTR in the cells as indicated by the presence of mature Band C, which is elevated by treatment with C18, as expected, and immature Band B (FIG. 25E, 25F).

To further assess the retained function in CFTR-Δ23 cells were treated with the two newly FDA-approved CF correctors. VX-661+VX-445 (FIG. 25G). Treatment with the triple modulator combination (VX-661. VX-445. VX-770) resulted in superior improvement of CFTR conductance, representing ˜20% of WT-CFTR function (FIG. 25H), providing evidential support that elimination of this exon retains function that is rescued further by CF modulators. Importantly, as these constructs encode CFTR cDNA, the effect of the PTC introduced by W1282X-CFTR on mRNA stability is not addressed by this assay.

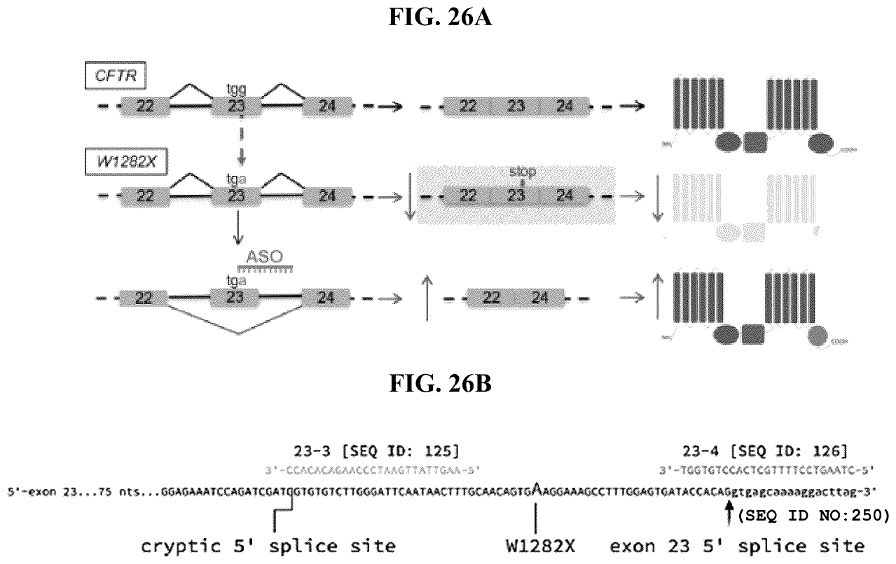

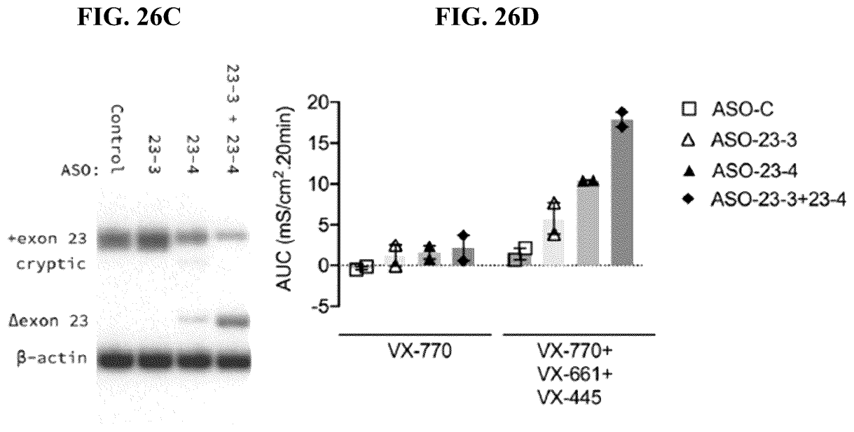

ASO-induced exon 23 skipping increases CFTR mRNA and recovers chloride channel activity in a CFTR W1282X immortalized human bronchial epithelial cell line. A major disease mechanism of the CFTR W1282X mutation is mRNA destabilization of due to targeting of the PTC-containing mRNA for degradation by the nonsense mediated decay pathway (FIG. 26A middle panel). Results with CFTR-Δ23 expression in FRT cells show that the isoform retains some CFTR function. An ASO designed to induce CFTR exon skipping and eliminate the PTC, could increase CFTR mRNA abundance resulting in translation of more semi-functional CFTR protein that would be responsive to further rescue by CF modulator drugs (FIG. 26A bottom panel).

ASOs were designed to basepair to the human CFTR exon 23 pre-mRNA and tested for their ability to induce exon 23 skipping via steric interference of the splicing machinery in an immortalized CFTR W1282X patient-derived bronchial epithelial cell line (16HBEge-W1282X-CFTR) (FIG. 26B). This cell line has minimal Cl-transport activity and CFTR mRNA levels are ˜20% of wild-type, due to NMD (Valley et al. 2019). Treatment of these cells with ASO-23-4 resulted in exon 23 skipping (FIG. 26C, 26D, Table 7).

ASO-23-4 strongly induced exon 23 skipping but also activated a cryptic 5′ splice site within exon 23. The mRNA produced from splicing at this cryptic site is out of frame, creating a nearby PTC. Blocking this cryptic site with another ASO could increase exon 23 skipping induced by ASO 23-4 by reducing competition for splicing by the cryptic site. Thus, ASO 23-3 was designed, which basepairs to the region comprising the cryptic splice site (FIG. 26B). Treatment with both ASO-23-3 to block the cryptic 5′ splice site and ASO-23-4 to block the natural 5′ splice site resulted in robust exon 23 skipping without cryptic splicing (FIG. 26C, Table 7).

To correlate the ASO-mediated increase in stable CFTR mRNA with CFTR function, it was tested whether ASO-23-4 could improve forskolin-stimulated conductance in 16HBEge-W1282X-CFTR clonal cell lines selected for high resistance (Clone 1=3F2). Cells were plated on trans-well filter plates and transfected on the apical surface with vehicle, ASO-C, ASO-23-3, ASO-23-4 or ASO-23-3 combined with ASO-23-4 after 4 days. After 48 hours, the transfection media was removed and replaced with media containing the correctors VX-661+VX-445 or vehicle (0.2% DMSO). Cells were treated for 24 hours and then assayed for conductance using a TECC-24 workstation. Transepithelial resistance (Rt) was recorded to calculate a conductance (Gt) attributable to forskolin-stimulated chloride secretion. Along with corrector treatments, the activity of the potentiator VX-770, an FDA-approved therapeutic for patients with CF, was tested. Both the correctors along with VX-770 make up the drug Trikafta, the most recent approved modulator therapy for CF patients. Inhibitor-172 (Inh-172), an inhibitor CFTR, was added 20 minutes after VX-770 to confirm that the measured conductance was a result of CFTR activity (FIG. 26D). Measurements were plotted as conductance traces and area under the curve (AUC) of the forskolin+V-770 test period was calculated to assess treatment effect. RNA was isolated after functional analysis to assess CFTR mRNA stability and exon 23 skipping.

Treatment with the ASO-23-4 alone resulted in a robust increase in conductance over the control treatments when combined with modulator treatment (FIG. 26D, Table 8). Treatment with ASO-23-3 alone did not increase conductance, but treatment with both ASO-23-3 and ASO-23-4 increased conductance above that achieved with treatment with ASO-23-4 alone (FIG. 26D, Table 8). The increase in conductance mediated by ASO treatment correlated with an increase in CFTR-exon 23 skipping (FIG. 26C. Table 7, Table 8). Together, these results reveal that the treatment of CFTR W1282X cells with a combination of ASO-23-3 and 23-4 induces robust exon 23 skipping and a significant increase in CFTR conductance. The combination of the two ASOs is more effective than cither ASO alone.

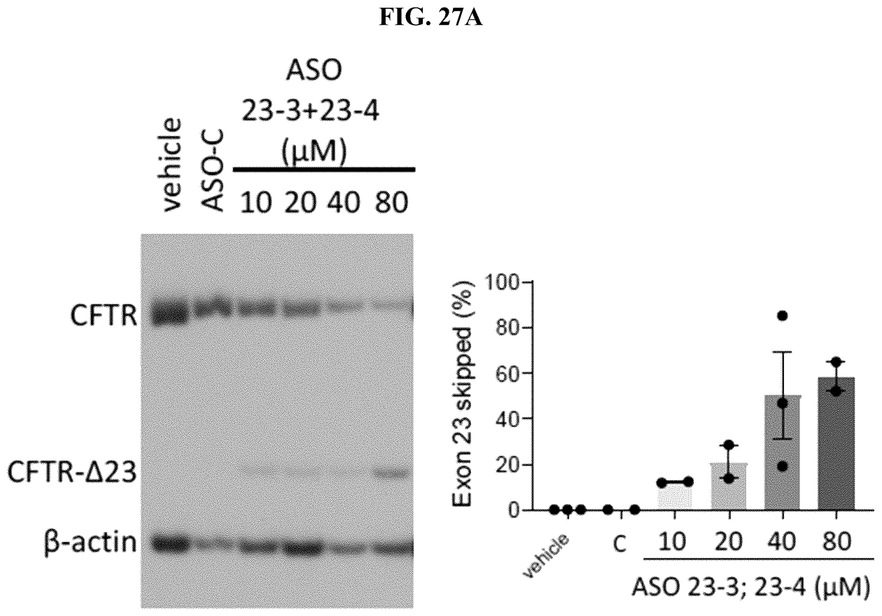

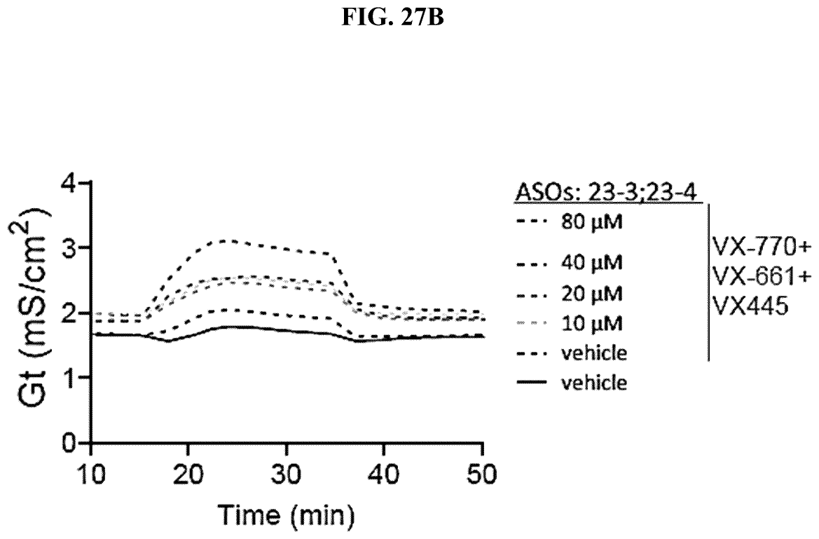

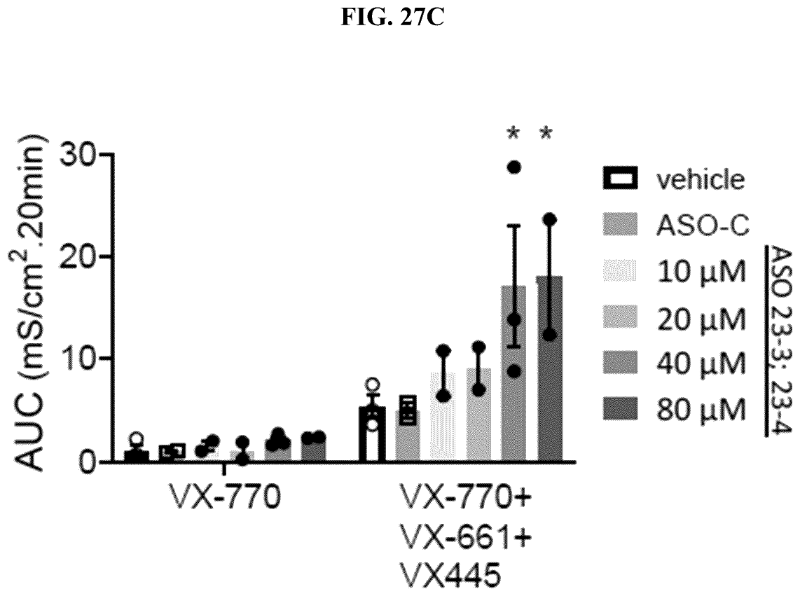

To further demonstrate the activity of the ASO-23-3 plus ASO-23-4 combination treatment, cells were treated with an increasing dose of the two ASOs in 16HBEge-W1282X-CFTR cells and tested conductance and splicing as described previously (FIG. 26). Increasing the concentration of each ASO delivered to the cells resulted in increasing exon 23 skipping (FIG. 27A, Table 9) and chloride conductance (FIG. 27B, 27C, Table 10) that correlated with the dose.

TABLE 6
Splice-switching Antisense Oligonucleotides,
Primers, and Probes

| | Sequence (5′-3′) |
|---|---|
| ASOs | |
| 23-1 | ATCCAGTTCTTCCCAAGAGGCCCAC |
| | (SEQ ID NO: 123) |
| 23-2 | AGCTGATAACAAAGTACTCTTCCCT |
| | (SEQ ID NO: 124) |
| 23-3 | AAGTTATTGAATCCCAAGACACACC |
| | (SEQ ID NO: 125) |
| 23-4 | CTAAGTCCTTTTGCTCACCTGTGGT |
| | (SEQ ID NO: 126) |
| Control (ASO-C) | CCTCTTACCTCAGTTACAATTTATA |
| | (SEQ ID NO: 151) |
| Primers | |
| 41: hCFTRex22F | CCAAACCATACAAGAAT |
| | (SEQ ID NO: 152) |
| 41: hCFTRex24R | GATCACTCCACTGTTCAT |
| | (SEQ ID NO: 153) |
| 43: hCFTR-F508F | TGGCACCATTAAAGAAAATATCATC |
| | TT (SEQ ID NO: 154) |
| 44: hCFTR-F508R | CTCAGTGTGATTCCACCTTCTC |
| | (SEQ ID NO: 155) |
| 45: hβ-actinFor | AAAGACCTGTACGCCAACAC |
| | (SEQ ID NO: 156) |
| 45: hβ-actinRev | GTCATACTCCTGCTTGCTGAT |
| | (SEQ ID NO: 157) |
| Probes | |
| hCFTR-F508 | 5.6-FAM/ACAGAAGCG/ZEN/ |
| | TCATCAAAGCATGCC/3IABkFQ |
| | (SEQ ID NO: 158) |
| hβ-actin | 5HEX/TCATCCATG/ZEN/ |
| | GTGAGCTGGCGG/3IABkFQ |
| | (SEQ ID NO: 159) |

TABLE 7
Exon 23 skipping quantitation from FIG. 26C
measured by RT-PCR analysis of RNA isolated
from immortalized CFTR W1282X human
bronchial epithelial cells (16HBEge-W1282X-
CFTR clone 3F2) treated with VX-770, VX-661,
VX445 in combination with indicated ASO(s).
The highest skipping is achieved with the
combination of SEQ ID: 125 with SEQ ID: 126.

| ASO | Exon 23 skipping (%) |
|---|---|
| ASO-C | 0 |
| ASO-23-3 (SEQ ID: 125) | 0 |
| ASO-23-4 (SEQ ID: 126) | 12 |
| ASO-23-3 (SEQ ID: 125) + | 79 |
| ASO-23-4 (SEQ ID: 126) | |

TABLE 8
Conductance measurements of immortalized CFTR
W1282X human bronchial epithelial cells (16HBEge-
W1282X-CFTR clone 3F2) treated with VX-770 alone
or VX-770, VX-661, VX445 in combination with
indicated ASO(s) from FIG. 26D. The highest
conductance is achieved with the combination of
SEQ ID: 125 with SEQ ID: 126.

| | AUC (mS/cm² · 20 min) | |
|---|---|---|
| | | VX-770 + |
| | | VX-661 + |
| ASO | VX-770 | VX-445 |
| ASO-C | −0.35 | 1.38 |
| ASO-23-3 (SEQ ID: 125) | 1.21 | 5.76 |
| ASO-23-4 (SEQ ID: 126) | 1.59 | 10.5 |
| ASO-23-3 (SEQ ID: 125) + | 2.13 | 17.9 |
| ASO-23-4 (SEQ ID: 126) | | |

TABLE 9
Exon 23 skipping quantitation from FIG. 27A
measured by RT-PCR analysis of RNA isolated from
immortalized CFTR W1282X human bronchial
epithelial cells (16HBEge-W1282X-CFTR clone 3F2)
treated with VX-770, VX-661, VX445 in combination
with indicated ASO(s).

| ASO | Exon 23 skipping (%) |
|---|---|
| vehicle | 0 |
| ASO-C | 0 |
| ASO-23-3; 23-4 10 μM | 12 |
| ASO-23-3; 23-4 20 μM | 21 |
| ASO-23-3; 23-4 40 μM | 51* |
| ASO-23-3; 23-4 80 μM | 59* |

*p < 0.05, One-way ANOVA with Dunnett's multiple comparison test compared to vehicle.

TABLE 10
Conductance measurements of immortalized CFTR
W1282X human bronchial epithelial cells (16HBEge-
W1282X-CFTR clone 3F2) treated with DMSO
(vehicle) or VX-770, VX-661, VX-445 in combination
with indicated ASO23-3 + 23-4 (SEQ ID: 125 with
SEQ ID: 126) from FIG. 27C.

| | AUC (mS/cm² · 20 min) | |
|---|---|---|
| | | VX-770 + |
| | | VX-661 + |
| ASO | DMSO | VX-445 |
| vehicle | 0.55 | 5.28 |
| ASO-C | 0.53 | 3.80 |
| ASO-23-3; 23-4 10 μM | 1.50 | 8.60 |
| ASO-23-3; 23-4 20 μM | 0.87 | 9.67 |
| ASO-23-3; 23-4 40 μM | 2.03 | 17.2** |
| ASO-23-3; 23-4 80 μM | 2.27 | 18.0*** |

**P < 0.005,
***P < 0.001, Two-way ANOVA analysis with Sidak's multiple comparison test.

### Example 10. Open Reading Frame Correction Using Antisense Oligonucleotides for the Treatment of Cystic Fibrosis

CFTR gene mutations that result in the introduction of premature termination codons (PTCs) are common in cystic fibrosis (CF). This mutation type causes a severe form of the disease, likely because of low CFTR mRNA expression as a result of nonsense-mediated mRNA decay (NMD), as well as the production of a non-functional, truncated CFTR protein. Current therapeutics for CF, which target residual protein function, are less effective in patients with these types of mutations, due in part to low CFTR protein levels. Splice-switching antisense oligonucleotides (ASOs) designed to induce skipping of exons in order to restore the mRNA open reading frame have shown therapeutic promise pre-clinically and clinically for a number of diseases. ASO-mediated skipping of CFTR exon 23 may recover CFTR activity associated with terminating mutations in the exon, including CFTR p. W1282X, the 5th most common mutation in CF. As shown herein, CFTR lacking the amino acids encoding exon 23 is partially functional and responsive to corrector and modulator drugs currently in clinical use. ASO-induced exon 23 skipping rescued CFTR expression and chloride current in primary human bronchial epithelial cells isolated from homozygote CFTR-W1282X patients. These results support the use of ASOs in treating CF patients with CFTR class I mutations in exon 23 that result in unstable CFTR mRNA and truncations of the CFTR protein.

Cystic fibrosis (CF) is an autosomal recessive genetic disease caused by mutations in the cystic fibrosis transmembrane conductance regulator (CFTR) gene. CFTR transports chloride and bicarbonate across the apical surface of epithelial cells. Loss of CFTR expression or function affects multiple organ systems, including the lungs, liver, pancreas, intestines, smooth muscle and heart. In the lung, CFTR-mediated chloride secretion and sodium absorption by the epithelial sodium channel regulate airway surface liquid hydration. Loss of CFTR causes disruption of mucociliary clearance, resulting in the proliferation of airway pathogens, chronic infection, inflammation, and bronchial damage.

Though there are over 2,000 variants in CFTR, most therapeutics that are clinically available or in development are designed for specific patient subpopulations with the most common mutations. Currently, there are four FDA-approved drugs that directly target CFTR function. These drugs are referred to as CFTR modulators. Ivacaftor (VX-770) potentiates function of CFTR by increasing the probability of channel opening for gating variants, lumacaftor (VX-809) and tezacaftor (VX-661) correct processing and trafficking of CFTR to the cell surface. A new corrector, elexacaftor (VX-445), works in combination with tezacaftor and ivacaftor. While drug development has recently expanded drastically there is a critical need for therapies to treat patients with rare CFTR mutations, in particular nonsense variants that create a premature termination codons (PTC) resulting in low CFTR expression.

One of the most common nonsense mutations associated with CF is CFTR p. W1282X (c.3846G>A). CFTR-W1282X is the fifth most common CF-causing mutation worldwide and the second most common class I mutation associated with the disease. This mutation results in a truncated CFTR (CFTR1281), removing ˜60% of the nucleotide binding domain 2 (NBD2) but retaining 1281 of the 1480 amino acids in the full-length protein. The truncated CFTR-W1282X protein has processing and/or gating defects but is responsive to potentiator and corrector treatment. However, CFTR-W1282X mRNA is degraded by nonsense mediated mRNA decay (NMD), leading to a decrease in mRNA and protein abundance, thereby limiting the effect of modulator drugs. Small molecule compounds that inhibit NMD have been shown to increase CFTR-W1282X expression but to date no effective drug candidates targeting NMD have been approved for use.

Antisense oligonucleotides (ASOs) are another possible therapeutic approach for treating CF caused by nonsense and frameshift mutations. ASOs are short oligonucleotides, chemically-modified to create stable, specific and long lasting drugs that can be designed to modulate pre-mRNA splicing. ASOs can be designed to block splicing and induce skipping of an exon, effectively removing it from the mRNA. This strategy can be useful as a potential therapeutic for CFTR-W1282X as amino acid 1282 resides in exon 23 which is a symmetrical exon that can be eliminated from the mRNA without disrupting the CFTR open reading frame. This approach would have a therapeutic benefit not only by producing a potentially functional protein isoform with a restored C-terminus, as suggested by biochemical and functional studies of CFTR, but also by eliminating the PTC, stabilizing the mRNA and increasing protein expression, thereby improving efficacy of CFTR modulators.

As demonstrated herein, a CFTR isoform lacking the amino acids encoded by exon 23 has partial activity when exposed to CFTR modulator drugs. A splice-switching ASO strategy that induces exon 23 skipping was identified and shown that ASO-mediated exon 23 skipping in CFTR-W1282X RNA, in both an immortalized human CFTR-W1282X bronchial epithelial cell line and primary epithelial cells isolated from CF patients homozygous for CFTR-W1282X, stabilizes the CFTR mRNA and recovers CFTR activity. Also provided herein is evidence that ASO-induced exon 23 skipping has partial allele specificity for CFTR-W1282X which could be advantageous in treating CF patients heterozygous for CFTR-W1282X and another mutation less responsive to current therapeutics.

### Results

CFTR mRNA Lacking Exon 23 Generates a Partially Active Protein.

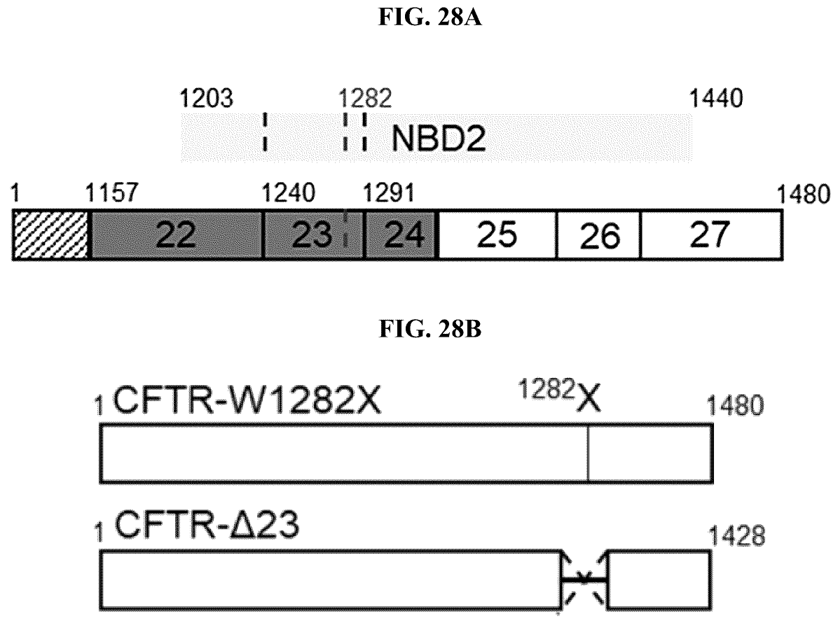

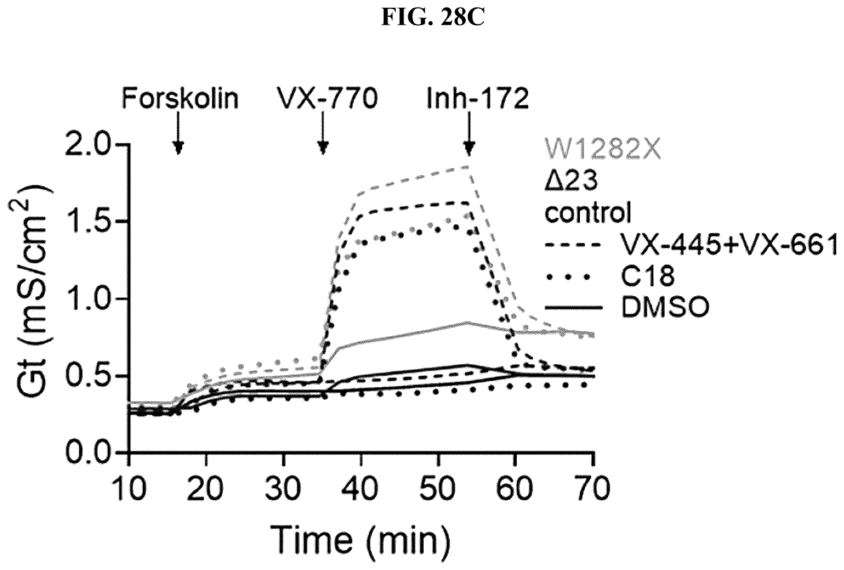

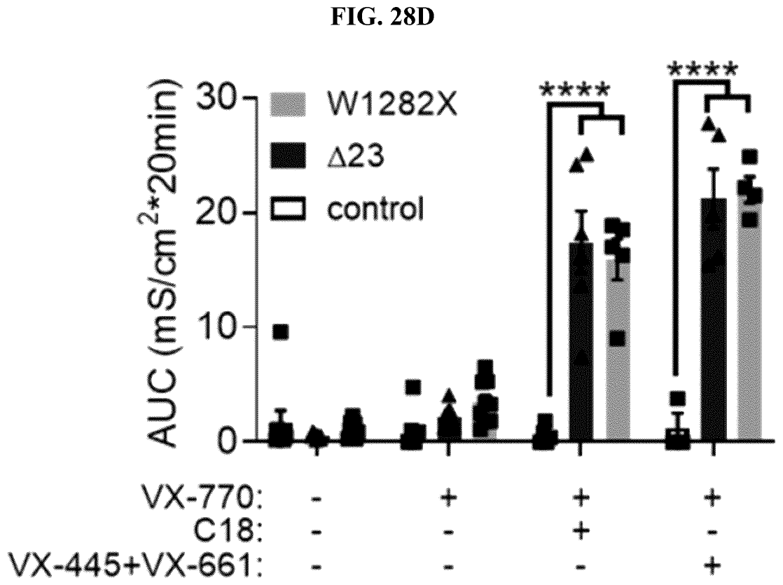

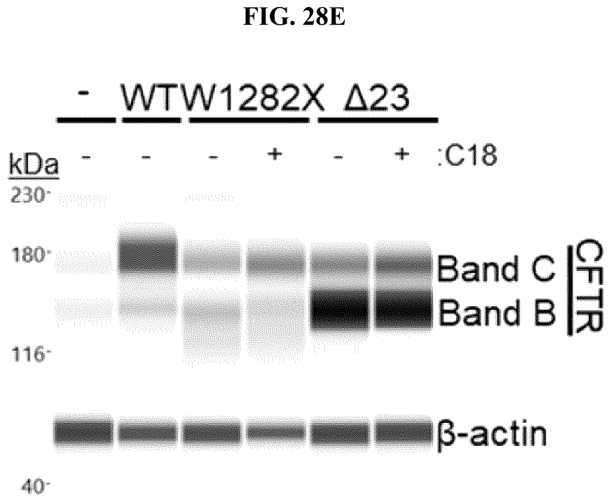

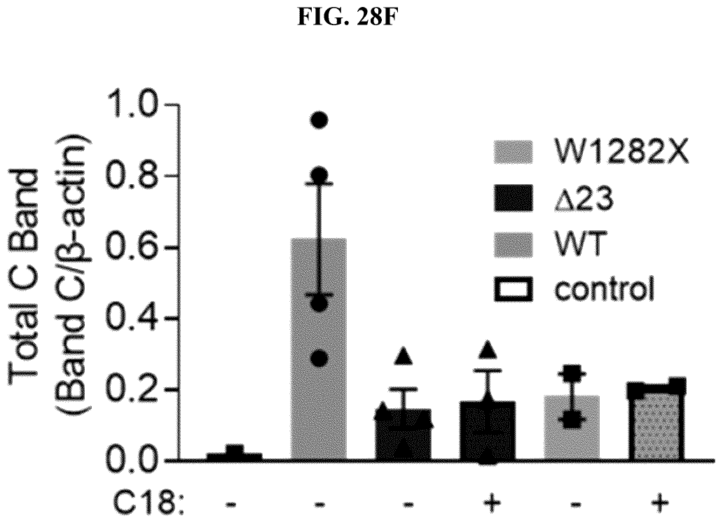

The CFTR-W1282X mutation resides within exon 23 of CFTR RNA. Exon 23 is a symmetrical exon that can be removed without disrupting the open reading frame (FIG. 28A). As a first step in determining whether correction of the CFTR-W1282X open reading frame by removing exon 23 might be therapeutic, it was tested whether an expressed CFTR isoform lacking the amino acids encoded by exon 23 had channel activity (FIG. 28B). FRT cells, which lack endogenous CFTR, were transfected with plasmids expressing CFTR without exon 23 (CFTR-Δ23) or with the W1282X mutation (CFTR-W1282X). Transepithelial resistance measurements were recorded from the cells after monolayers formed and transepithelial conductance (Gt=1/Rt) attributed to forskolin-stimulated activation of CFTR and inhibition by CFTR inhibitor. Inh-172, were calculated. Measurements were plotted as conductance traces (FIG. 28C) and the area under the curve (AUC) was calculated for comparison (FIG. 28D). To test the responsiveness of CFTR-W1282X and CFTR-Δ23 to CFTR modulators known to increase CFTR-W1282X function, the cells were treated with VX-770 and C18 (VRT-534), an analog of the corrector VX-809, or VX-445+VX-661. CFTR-specific activity was undetectable in untreated or VX-770-treated cells. In contrast, both CFTR-Δ23 and CFTR-W1282X had similar significant increases in activity following corrector and potentiator treatment (FIGS. 28C and 28D). This increase in functional activity corresponded with an increase in CFTR protein as indicated by an increase in the fully glycosylated CFTR isoform (Band C) and the core glycosylated isoform (Band B) (FIGS. 28E and 28F). These results demonstrate that CFTR-Δ23 has functional activity in the presence of CFTR modulator drugs.

A splice-switching ASO induces exon 23 skipping and increases CFTR mRNA and chloride channel activity in a CFTR-W1282X immortalized human bronchial epithelial cell line.

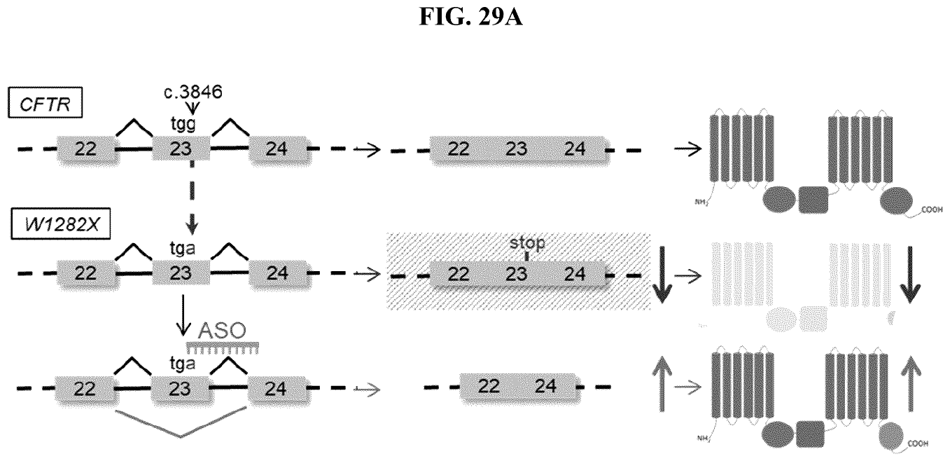

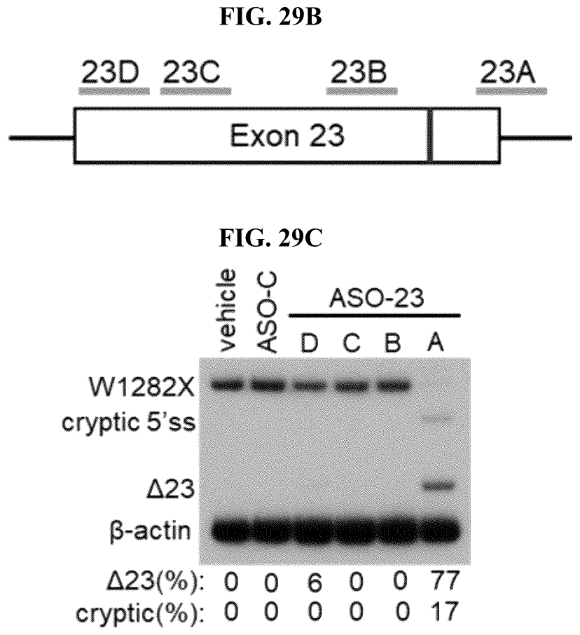

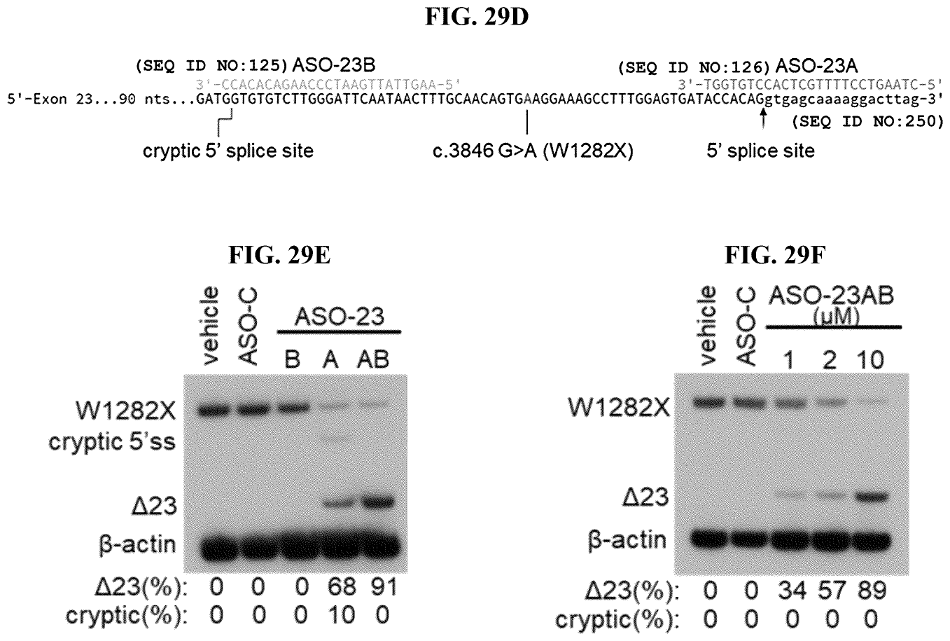

Splice-switching ASOs are a therapeutic platform that can be used to induce exon 23 skipping to stabilize CFTR-W1282X mRNA and increase abundance of the partially functional CFTR-Δ23 protein isoform (FIG. 29A). Four ASOs designed to base-pair to human CFTR exon 23 pre-mRNA and induce exon 23 skipping via a steric block of the splicing machinery were tested (FIG. 29B). ASO-23A, which basepairs to the 5′ splice site (FIGS. 29B and 29D), induced exon 23 skipping when transfected into an immortalized patient-derived bronchial epithelial cell line expressing CFTR-W1282X (CFF16HBEge-W1282X) (FIG. 29C). ASO-23A also induced splicing at a cryptic 5′ splice site within exon 23, which results in out-of-frame mRNA. To reduce the use of this cryptic splice site and maximize exon 23 skipping, cells were co-transfected with ASO-23A and another ASO. ASO-23B, which blocks the cryptic splice site (FIG. 29D). Treatment of cells with ASO-23A and ASO-23B (ASO-23AB) eliminated cryptic splice site use and resulted in a dose-dependent increase in exon 23 skipping (FIGS. 29E and 29F).

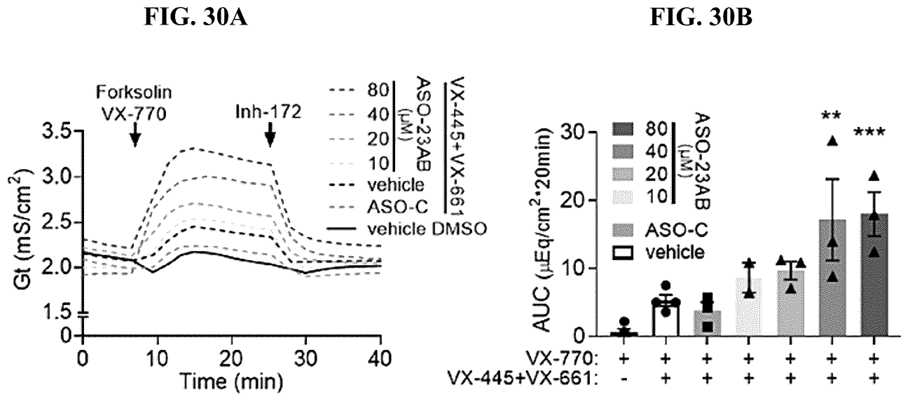

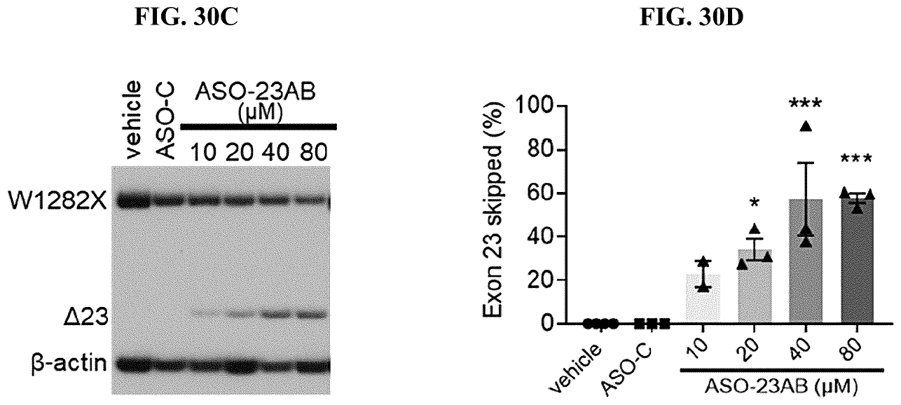

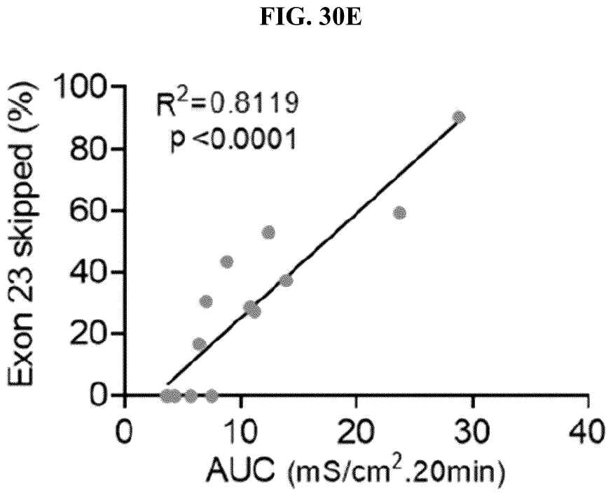

It was next tested whether ASO-23AB treatment could increase conductance in the immortalized hBE CFTR-W1282X cell lines. ASO-23AB treatment resulted in a significant increase in conductance when modulators were present compared to controls (FIGS. 30A and 30B). The conductance increased in an ASO dose-dependent manner (FIG. 30B). This increase in activity corresponded with an increase in exon 23 skipping (FIGS. 30C and 30D). There was a positive correlation between ASO-induced exon 23 skipping and conductance (FIG. 30E).

### ASO-Induced Exon Skipping Rescues Chloride Currents in Homozygous CFTR-W1282X Patient-Derived Bronchial Epithelial Cells.

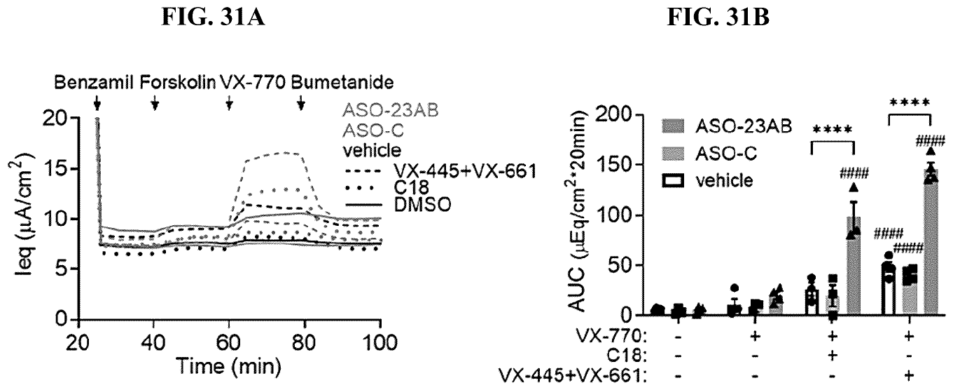

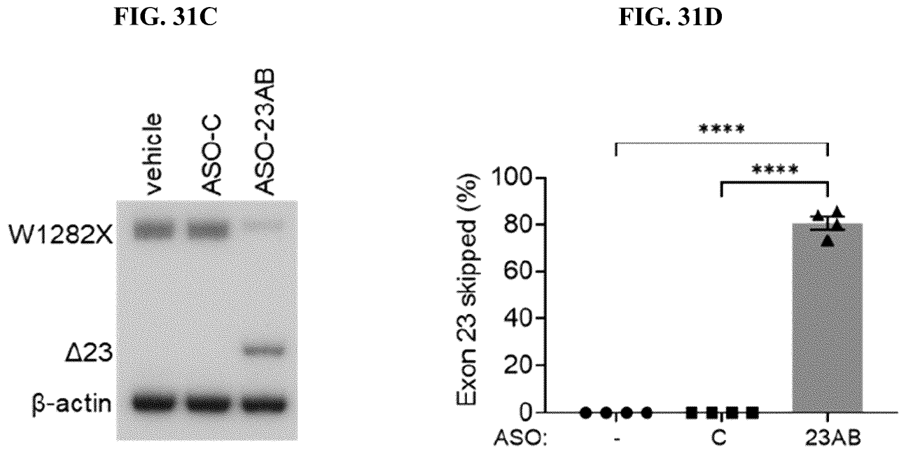

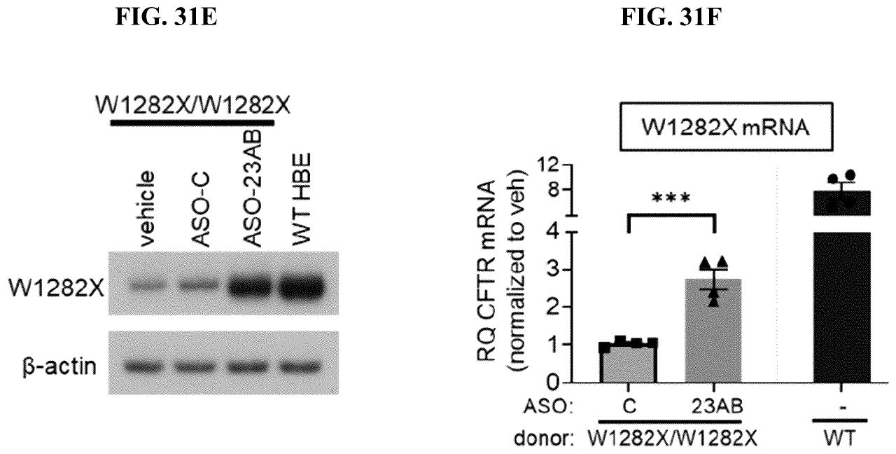

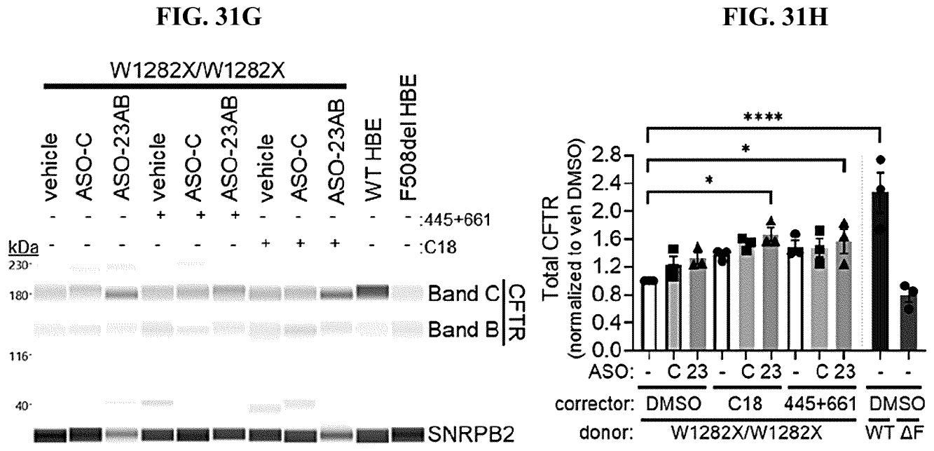

To further assess the therapeutic potential of ASO-induced exon skipping in correcting the CFTR-W1282X mutation the effects of ASO treatment on channel activity was analyzed in differentiated primary human bronchial epithelial (hBE) cells isolated from a CF patient homozygous for CFTR-W1282X. This cell-based model is the gold-standard for pre-clinical testing of CF therapeutics as the functional responses to drugs in this assay has been shown to accurately predict efficacy in the clinic. Transepithelial voltage (Vt) and resistance (Rt) was recorded to calculate an equivalent current (Ieq=Vt/Rt). Without ASO treatment, only the combination treatment of VX-770. VX-445 and VX-661 had a significant effect on chloride secretion in the cells (FIGS. 31A and 31B). ASO-23AB treatment in combination with VX-770+C18, or VX-770+VX-445+VX-661, resulted in a ˜5-fold and 3-fold increase in chloride secretion, respectively, compared to either modulator treatment alone (FIGS. 31A and 31B).

This functional rescue by ASO-23AB treatment was accompanied by a significant induction of exon 23 skipping (FIGS. 31C and 31D). ASO-23AB treatment resulted in a 3-fold increase in total CFTR mRNA compared to untreated samples, a level that is ˜30% of mRNA levels in wildtype non-CF donor hBE cells (FIGS. 31E and 31F). The rescue of total CFTR RNA expression is indicative of a stabilization of mRNA as a result of elimination of the PTC introduced by the CFTR-W1282X mutation in exon 23. ASO treatment also resulted in an increase in CFTR protein (FIGS. 31G and 31H). The stabilization of CFTR-W1282X mRNA and increase in CFTR protein expression correlates with the rescue of chloride secretion in these patient cells, predictive of a potential therapeutic effect of ASO-23AB treatment over current modulator drugs for patients homozygous for CFTR-W1282X.

### Activity and Allele-Specificity of ASOs in Patient-Derived Bronchial Epithelial Cells Compound Heterozygous for CFTR-W1282X and F508del.

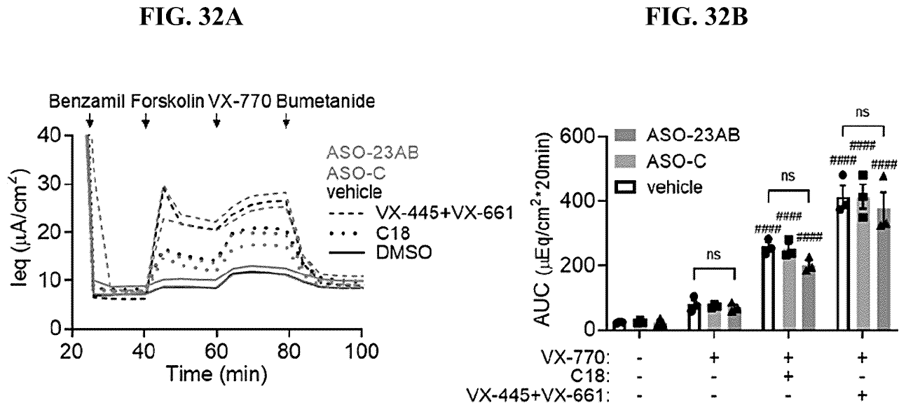

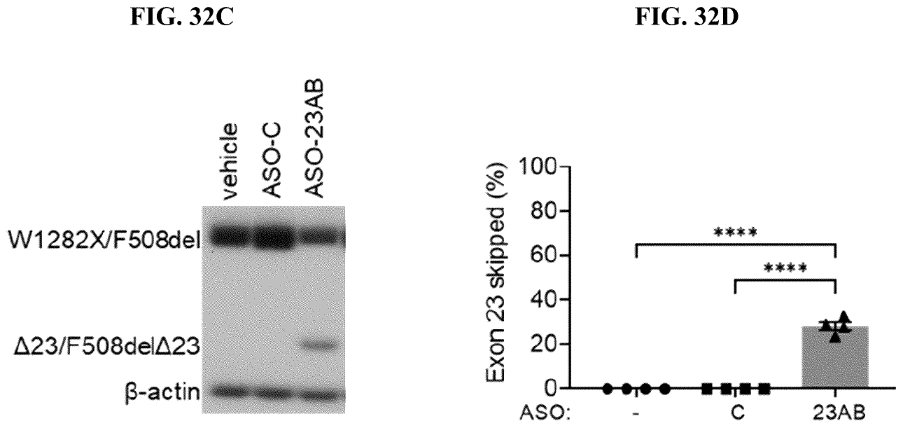

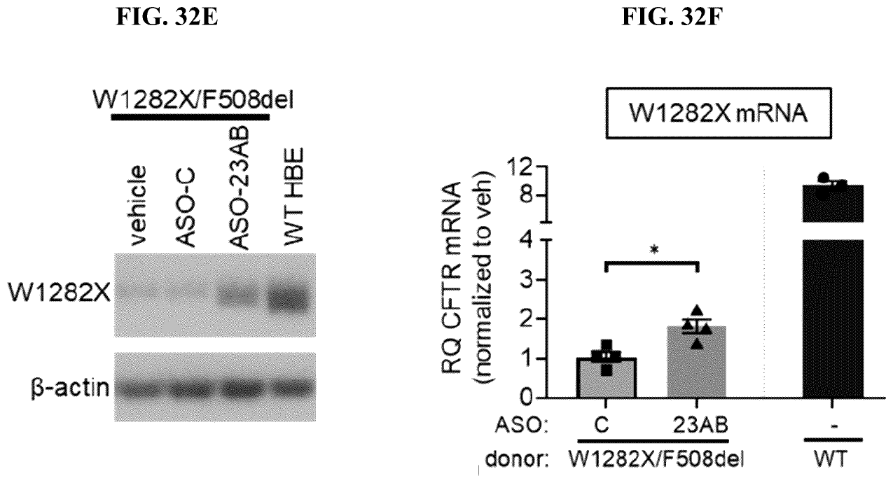

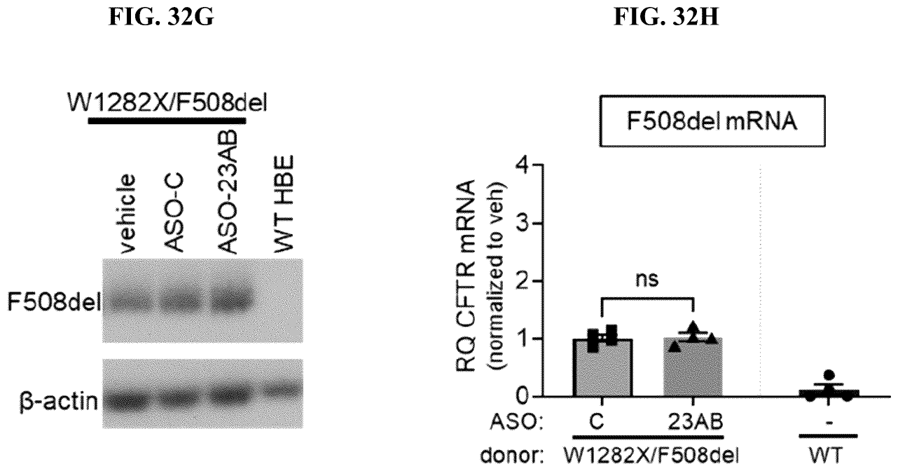

Many CF patients with the CFTR-W1282X mutation are compound heterozygotes, with a different mutation, most commonly CFTR-F508del, in the other CFTR allele. This second allele would also be a target of ASO-induced exon 23 skipping and would result in a CFTR protein with the original mutation and a deletion of exon 23. To test the effect of ASO-induced exon 23 skipping on mRNA from CFTR mutations commonly found with CFTR-W1282X in compound heterozygotes, primary hBE cells isolated from a CF patient with the CFTR-W1282X and CFTR-F508del mutations were treated with ASO-23AB and measured chloride secretion (FIG. 32A).

In cells from this patient, both modulator combinations of VX-770+C18, and VX-770+VX-445+VX-661, resulted in significant recovery of chloride secretion, as expected given that CFTR-F508del is known to be responsive to each drug (FIG. 32B). ASO-23AB treatment had no significant effect on this rescue, with cells showing no increase or decrease in potentiator and corrector response (FIG. 32B).

Analysis of RNA splicing revealed a significant induction of exon 23 skipping with ASO treatment compared to the controls. However, exon 23 skipping was considerably lower (25% of total RNA) (FIGS. 32C and 32D) than the skipping obtained in CFTR-W1282X homozygous donor cells (80% of total RNA) (FIGS. 31C and 31D). These results suggest that exon 23 skipping of RNA from CFTR-F508del may be less efficient.

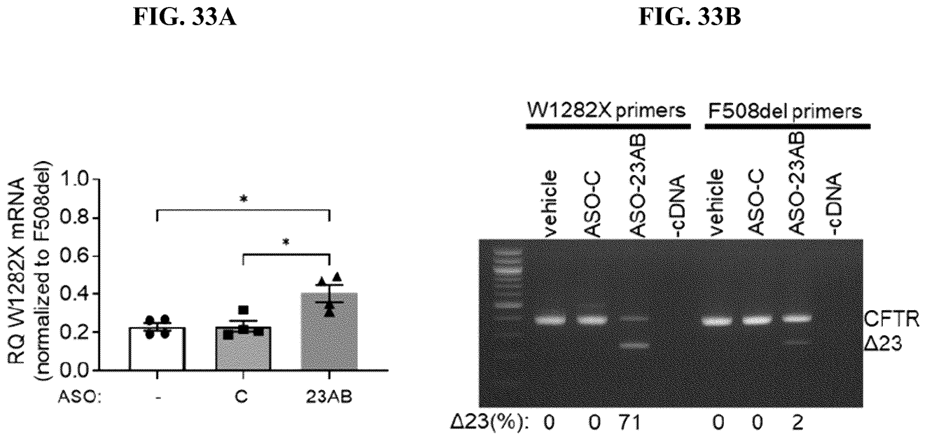

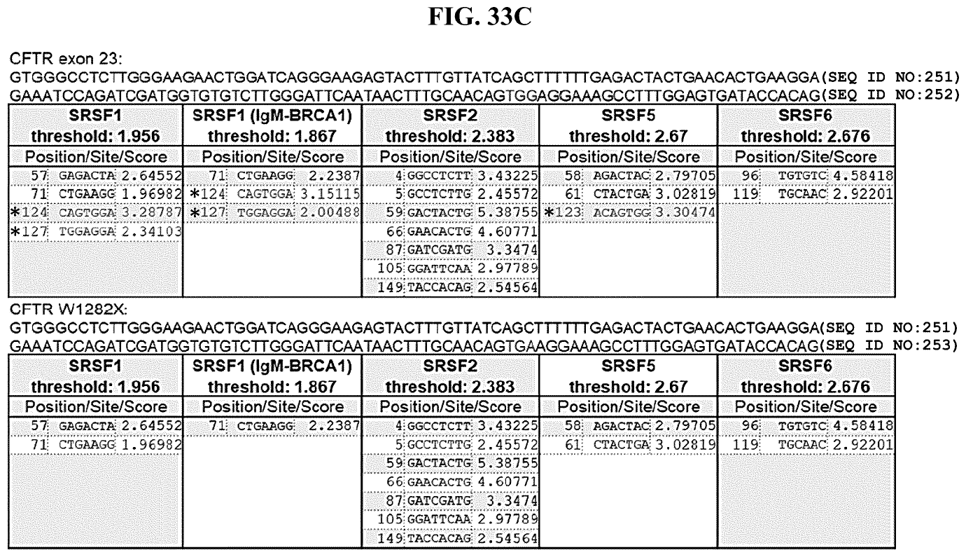

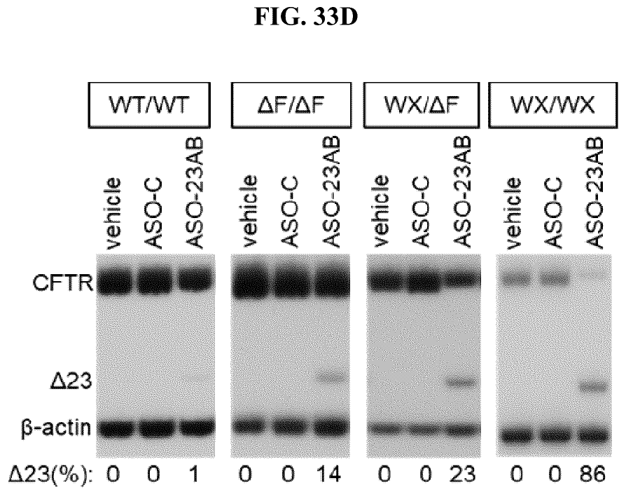

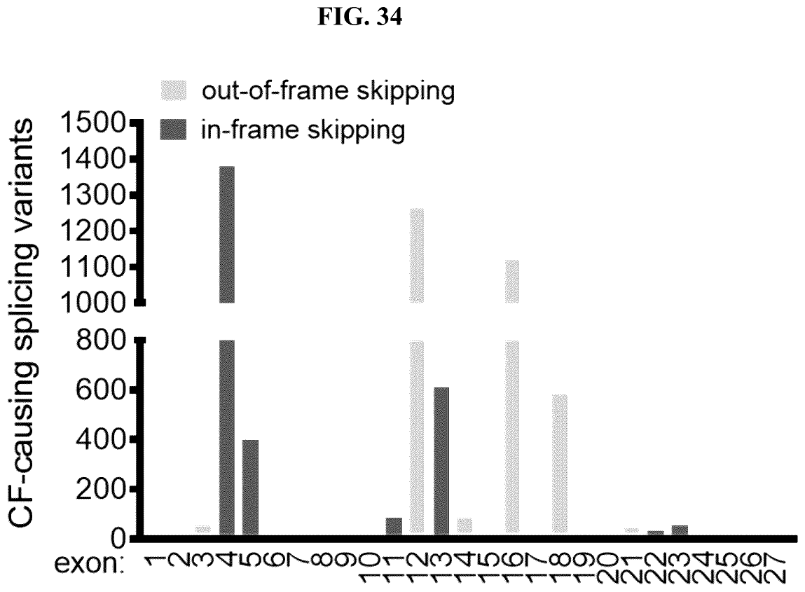

To analyze the effect of ASO-23AB on exon 23 from mRNA derived from each allele specifically, primers were designed to anneal at the F508del mutation site and specifically amplify either non-F508del or F508del mRNA (Table 11). When comparing the baseline expression of mRNA from each allele, RNA derived from the CFTR-W1282X allele was only 20% of that expressed from the CFTR-F508del allele (FIG. 33A) most likely due to transcript degradation by nonsense mediated decay. ASO treatment significantly increased this expression to ˜40% of that generated from the CFTR-F508del allele (FIG. 33A). Analysis of RNA from each allele separately revealed a 2-fold increase in CFTR transcripts from CFTR-W1282X, similar to levels achieved in the homozygous donor and up to 20% of wild-type CFTR expression (FIGS. 32E and 32F). In contrast, the ASO had no significant effect on total CFTR mRNA expression from the CFTR-F508del allele (FIGS. 32G and 32H).

TABLE 11
Sequences

| | Sequence (5′ to 3′) |
|---|---|
| ASOs | |
| ASO-23A | CTAAGTCCTTTTGCTCACCTGTGGT (SEQ ID NO: 126) |
| ASO-23B | AAGTTATTGAATCCCAAGACACACC (SEQ ID NO: 125) |
| ASO-23C | AGCTGATAACAAAGTACTCTTCCCT (SEQ ID NO: 124) |
| ASO-23D | ATCCAGTTCTTCCCAAGAGGCCCAC (SEQ ID NO: 123) |
| ASO-C | CCTCTTACCTCAGTTACAATTTATA (SEQ ID NO: 151) |
| Primers | |
| 1: HCAI-CFTRde123R | GCGCTGGCCGGGGCTGAT (SEQ ID NO: 160) |
| 2: HCAI-CFTRde123F | AAGGTGTTCATCTTCAGCGGCACCTTC (SEQ ID NO: 161) |
| 3: HCAI-CFTRWXF | TGCAGCAGTGACGCAAAGGCCTT (SEQ ID NO: 162) |
| 4: HCAI-CFTRWXR | GGGTGATGCTGTCCCAGC (SEQ ID NO: 163) |
| 5: hCFTR-ex11ΔFF | GCCTGGCACCATTAAAGAAAATATCATTGG (SEQ ID NO: 164) |
| 6: hCFTR-exl1F | GCCTGGCACCATTAAAGAAAATATCATCTT (SEQ ID NO: 165) |
| 7: hCFTR-ex14R | TCCAGGAGACAGGAGCATCT (SEQ ID NO: 166) |
| 8: hCFTR-ex22F | CCAAACCATACAAGAAT (SEQ ID NO: 152) |
| 9: hCFTR-ex24R | GATCACTCCACTGTTCAT (SEQ ID NO: 153) |
| 10: hCFTR-ex25R | GTTCTATCACAGATCTGAG (SEQ ID NO: 167) |
| 11: qhCFTR-ex11WTF | TGGCACCATTAAAGAAAATATCATCTT (SEQ ID NO: 154) |
| 12: qhCFTR-ex12WTR | CTCAGTGTGATTCCACCTTCTC (SEQ ID NO: 155) |
| 13: qhCFTR-exl1ΔFF | GGCACCATTAAAGAAAATATCATTGG (SEQ ID NO: 168) |
| 14: qhCFIR-exl2ΔFR | CTCAGTGTGATTCCACCTTCT (SEQ ID NO: 169) |
| 15: hβ-actinFor | AAAGACCTGTACGCCAACAC (SEQ ID NO: 156) |
| 16: hβ-actinRev | GTCATACTCCTGCTTGCTGAT (SEQ ID NO: 157) |
| 17: qhHPRT1For | GCGATGTCAATAGGACTCCAG (SEQ ID NO: 170) |
| 18: qhHPRT1Rev | TTGTTGTAGGATATGCCCTTGA (SEQ ID NO: 171) |
| Probes | |
| hCFTR-DF508 | /56-FAM/ACAGAAGCG/ZEN/TCATCAAAGCATGCC/3IABkFQ/ |
| | (SEQ ID NO: 172) |
| hCFTR-F508 | /5.6-FAM/ACAGAAGCG/ZEN/TCATCAAAGCATGCC/3IABkFQ/ |
| | (SEQ ID NO: 158) |
| hHPRT1 | /5HEX/AGCCTAAGA/ZEN/TGAGAGTTCAAGTTGAGTTTGG/ |
| | 3IABkFQ/ (SEQ ID NO: 173) |

To further investigate the reason for an increase in total mRNA from the CFTR-W1282X allele with ASO treatment, yet a large reduction in total exon 23 skipping compared to the homozygous donor cells, ASO-induced exon 23 skipping from each allele was analyzed using allele specific primers. This analysis revealed an increase in exon skipping in mRNA from the CFTR-W1282X allele (71%) compared to transcripts from the CFTR-F508del allele (2%), suggesting that ASO treatment has a greater effect on CFTR-W1282X than on CFTR-F508del (FIG. 33B). Comparative sequence analysis of exon 23 indicated that binding sites for several splicing proteins are eliminated by the G>A change in W1282X compared to wild-type CFTR (ESEfinder3.0) (FIG. 33C). The elimination of these splicing enhancer cis-acting sequences may weaken splicing to the exon and make the ASO more effective in inducing skipping. In fact, ASO treatment in non-CF and CFTR-F508del homozygous donor cells in comparison to donor cells with either one or two copies of CFTR-W1282X showed an increase in ASO-induced exon 23 skipping that correlated with the number of CFTR-W1282X alleles (FIG. 33D). This result suggests ASO-23AB may be more effective at inducing exon 23 skipping in CFTR-W1282X RNA, which could be advantageous in treating CF patients compound heterozygous for CFTR-W1282X and another CFTR mutation less responsive to modulator treatment.

### DISCUSSION

Despite clinical success, the use of ASOs to correct the translational open reading frame and recover gene expression in diseases caused by frameshift or nonsense mutations resulting in PTCs has not been extensively explored as a therapeutic approach. These types of mutations are the most common disease-causing mutations and account for ˜20% of disease-associated mutations in cystic fibrosis. As demonstrated herein, elimination of the relatively common nonsense mutation, CFTR-W1282X, by ASO-induced skipping of CFTR exon 23, which encodes the mutation, recovers CFTR expression. The activity of this CFTR isoform, lacking 52 amino acids, requires CFTR modulator drugs that are currently used to treat CF patients. Thus, this ASO approach in combination with current CF drugs offers a potential therapeutic for individuals with the CFTR-W1282X mutation and opens the door for similar strategies to treat other terminating mutations, both in CF and other diseases.

Gene mutations that result in PTCs are challenging to treat because they not only encode a truncated protein product, but the mRNA intermediate is a target of NMD, a cellular quality-control mechanism whereby mRNA with PTCs are degraded. Studies have shown that, if produced at sufficient levels, CFTR-W1282X protein is responsive to current CFTR modulator therapies but because stop mutations in CFTR result in a dramatic loss of CFTR expression, CF patients are not usually responsive to these drugs. Approaches are being pursued to identify molecules that stabilize mRNA by blocking NMD but, because NMD is an important mechanism regulating gene expression, any approach must avoid global inhibition of NMD which would likely have toxic effects. Small molecules that promote translational readthrough of termination codons are also being explored as potential treatments for PTC mutations, including CF. However, effects from the long-term use of these drugs has raised concerns. Though these approaches may hold promise, none are specific to CFTR directly, and to date none have been approved for use in CF patients. More recently, gene-specific suppression of NMD, has been explored as a promising approach to overcome potential risk of global NMD knockdown.

Using ASOs to remove exons encoding stop codons to correct the translational open reading frame has broad applications for addressing terminating mutations. ASO-mediated reading frame correction via induced skipping of symmetrical exons has shown promise in the FDA-approved ASOs targeting PTCs in Duchenne's Muscular Dystrophy and also in pre-clinical studies in mice for the treatment of diseases such as CLN3 Batten. A critical requirement for this approach is that the induced protein isoform must retain partial function. As shown herein, CFTR-Δ23 had significant CAMP-activated conductance responses that were further enhanced by modulator treatments (FIG. 28). Exon 23 encodes amino acids near the C-terminus of CFTR including a portion of the nucleotide-binding domain 2 (NBD2). The retained function of CFTR-Δ23 is consistent with previous reports that truncation at NBD2 results in a CFTR protein that is trafficked to the cell surface, albeit with deleterious effects on channel gating. The result also aligns with data showing that the CFTR-W1282X mutation is responsive to modulator therapies (FIG. 28). Notably, though truncation of CFTR at NBD2 results in some retained function, domains at the C-terminus are important for stability and gating and these domains are preserved when exon 23 is skipped.

The clinical potential of ASO delivery to the respiratory system, one of the primary targets for CF therapeutics, has been demonstrated for asthma and other inflammatory lung conditions. Naked ASOs have been successfully delivered to the lung, where they access multiple cell types including cells which express CFTR. Acrosolization of ASOs have shown promise in delivery in both a CF-like lung disease model in mice as well as CF patients. Additionally, ASOs have been shown to have long-lasting effects in primary human bronchial epithelial cells isolated from CF-patients, and in vivo, with treatment durations lasting weeks to months before further dosing is needed in patients. In addition, as patients with class I mutations tend to have disease phenotypes in other organs including the digestive system, systemic delivery of ASOs may also be considered. Intravenous and subcutaneous injections of ASOs are currently used to treat patients and bioavailable ASO formulations that target the gut epithelia have shown some potential.

Unlike other therapeutics targeting global NMD or inducing translational readthrough, the ASOs disclosed herein target CFTR transcripts specifically to induce skipping of exon 23 (FIG. 29). ASO treatment in immortalized and patient-derived hBE cells expressing CFTR-W1282X resulted in a dose-dependent increase in exon 23 skipping that correlated with an increase in function (FIGS. 30 and 31). Further, assessment of mRNA from ASO-treated primary hBE cells from a patient with CFTR-W1282X and CFTR-F508del revealed partial allele specificity of ASO induced exon 23 skipping for CFTR-W1282X (FIG. 33), potentially broadening the scope of this ASO strategy to CF patients heterozygous for CFTR-W1282X and another CFTR mutation less responsive to current modulator therapies. Despite effectively inducing skipping of CFTR-W1282X mRNA, ASO-induced exon 23 skipping did not have an improved effect on function compared to modulator treatment alone (FIG. 32). It is possible that a functional ceiling is achieved with modulator treatment in cells from this compound heterozygous donor, and additional expression from the CFTR-W1282X allele does not increase chloride current above what was already achieved with modulator rescue on CFTR-F508del, for which the modulators were developed. There have been a number of studies identifying other corrector and potentiator drugs superior to these modulators in the context of CFTR-W1282X. The effect of these new modulators was not tested along with ASO treatment here, but they have been shown to be effective in rescuing CFTR-W1282X activity in conjunction with other readthrough compounds that enhance CFTR-W1282X expression. Future studies may reveal better modulator/ASO combinations for treating CFTR-W1282X compound heterozygotes. Overall, the results herein support the use of ASO treatment in combination with approved CF modulators as an effective treatment option for CF patients with class I mutations within symmetrical exons.

### Materials and Methods

Expression plasmids: CFTR-Δ23 and CFTR-W1282X were created from the synthetic CFTR high codon adaption index (HCAI) construct subcloned in the pcDNA3.1/Neo (+) vector using the Q5 Site-Directed Mutagenesis Kit (NEB) with primers flanking each exon (Table 11). All plasmids were sequenced to confirm mutations. Plasmids were stably transfected into Fischer Rat Thyroid (FRT) cells in 6-well plates using lipofectamine LTX (Thermo Fisher) and OptiMEM (Thermo Fisher) for 48 hours. Cells were transferred to T75 flasks and clonal cell lines were selected with G418 (300 μg/ml) for one week. After selection cells were maintained in media supplemented with G418 (150 μg/ml).

Cells and culture conditions: FRT cell lines were cultured in F12 Coon's modification media (Sigma, F6636) supplemented with 10% FBS and 1% Penicillin-Streptomycin (PenStrep). 16hBEge-W1282X cell lines were obtained from the Cystic Fibrosis Foundation (CFF) and cultured according to their instructions in MEM media. Single-cell clones of the original CFF16hBEge-W1282X cell line were created to select for high resistance clonal cell lines. One clonal cell line, CFF16hBEge-W1282X-SCC: 3F2, was selected for analysis. Primary human bronchial epithelial cells (hBE) isolated from CF patients homozygous for CFTR-W1282X (patient code HBEU10014) and compound heterozygous for CFTR-W1282X and CFTR-F508del (patient code HBEND12112) were also obtained from the CFF. For functional analysis cells were differentiated by plating on Costar 24-well high-throughput screening filter plates (0.4 μM pore size, Polyester, Corning, catalog #CLS3397). FRT and 16hBE cells were grown in a liquid/liquid interface (180 μl apical/700 μl basolateral) in a 37° C. incubator with 90% humidity and 5% CO₂ for one week. Primary hBE cells were differentiated in an air/liquid interface for five weeks. Media was replaced three times a week.

Antisense oligonucleotides: Splice-switching antisense oligonucleotides are 25-mer phosphorodiamidate morpholino oligomers (Gene-Tools, LLC) (Table 11). A non-targeting PMO was used as a negative control, ASO-C. (Gene Tools, standard control oligo). ASOs were formulated in sterile water.

ASO cell transfection: For splicing analysis CFF16hBEge-W1282X-SCC: 3F2 clones were transfected with ASOs at indicated concentrations on 24-well plates in Minimum Essential Medium (MEM) media supplemented with 10% FBS and 1% PenStrep. Cells were transfected using Endo-Porter (Gene-Tools. 6 μl/ml) for 48 hours.

For functional analysis CFF16HBEge-W1282X-SCC: 3F2 cells were transfected on filter plates four days post plating. Cells were transfected with ASOs apically in 100 μl of complete MEM media with Endo-Porter at indicated concentrations for 48 hours.

Primary hBE cells were transfected after differentiation on filter plates as previously described (Michaels et al., (2020) Antisense oligonucleotide-mediated correction of CFTR splicing improves chloride secretion in cystic fibrosis patient-derived bronchial epithelial cells. Nucleic Acids Res., 48:7454-7467). Briefly, cells were transfected with ASO in an apical hypo-osmotic solution for 1 hour. The solution was removed, and the cells were treated again in DPBS for 4 days until functional analysis.

RNA isolation and RT-PCR: RNA was extracted from cells using TRIzol according to manufacturer instructions (Thermo Fisher Scientific). Reverse transcription was performed on total RNA using the GoScript Reverse Transcription System with an oligo-dT primer (Promega). Splicing was analyzed by radiolabeled PCR of resulting cDNA using GoTaq Green (Promega) spiked with α-32P-deoxycytidine triphosphate (dCTP). Primers for amplification are reported in Table 11. Reaction products were run on a 6% non-denaturing polyacrylamide gel and quantified using a Typhoon 7000 phosphorimager (GE Healthcare) and ImageJ software.

Real-time qPCR: Real-time qPCR was performed with PrimeTime Gene Expression Master Mix and PrimeTime qPCR probe assay kits human non-F508del-CFTR (IDT, qhCFTR-cx11WTF, qhCFTR-cx12WTR, hCFTR-F508), and human F508del-CFTR (IDT, qhCFTR-cx11AFF, qhCFTR-ex12ΔFR, hCFTR-DF508) transcripts were normalized to human HPRT1 (IDT. Hs.PT.58v.45621572) (Table 11). All reactions were analyzed in triplicate on 96-well plates and averaged together to comprise one replicate. Real-time PCR was performed on an Applied Biosystems (ABI) ViiA 7 Real-Time PCR System. Results were analyzed by the ΔΔCT method.

Protein isolation and automated western analysis: Cell lysates for immunoblot analysis were prepared from cells after functional analysis using NP-40 lysis buffer (1% Igepal. 150 mM NaCl. 50 mM Tris-HCl pH7.6) supplemented with 1× protease inhibitor cocktail (Sigma-Aldrich, cat #11836170001). Protein concentration was measured using a Coomassie (Bradford) protein assay (Thermo Fisher, cat #23200). Cell lysates were prepared using the sample preparation kit (Protein Simple) for an automated capillary western blot system. WES System (Protein Simple). Cell lysates were mixed with 0.1× sample buffer and 5× fluorescent master mix for a final protein lysate concentration of 0.2 mg/ml (FRTs) or 1.5 mg/ml (hBEs). Samples were incubated at room temperature for 20 minutes and then combined with biotinylated protein size markers, primary antibodies against CFTR 432 (FRTs). 570+450 (hBEs) (Riordan lab UNC. Cystic Fibrosis Foundation, diluted 1:100, or 1:50+1:200, with milk-free antibody diluent). β-actin (C4. Santa Cruz Biotechnology, diluted 1:50 with milk-free antibody diluent), and SNRBP2 (4G3, provided by the Krainer Lab: B″, diluted 1:2000 with milk-free antibody diluent), horseradish peroxidase (HRP)-conjugated secondary antibodies, chemiluminescence substrate and wash buffer and dispensed into respective wells of the assay plate and placed in WES apparatus. Samples were run in duplicate or triplicate. Signal intensity (area) of the protein was normalized to the peak area of the loading control C4. β-actin (FRT) or B″. SNRPB2 (hBE). Quantitative analysis of the CFTR B and C-bands was performed using Compass software (Protein Simple).

Automated conductance and equivalent current assay: Stably transfected FRT cells. 16HBEgc-W1282X-SCC: 3F2, and primary hBE cells were treated with C18 (6 μM) (VRT-534. VX-809 analog). VX-445+VX-661 (3 μM+3.5 μM FRT. 1 μM+3 μM 16hBE and primary hBE) or vehicle (equivalent DMSO) at 37° C. (33). Twenty-four hours later the cells were switched from differentiation media to HEPES-buffered (pH 7.4) F12 Coon's modification media (Sigma. F6636) apically and basolaterally and allowed to equilibrate for one hour at 37° C. without CO₂. To obtain the conductance measurements, the transepithelial resistance was recorded at 37° C. with a 24-channel TECC robotic system (EP Design, Belgium) as previously described (Vu et al . . . (2017) Fatty Acid Cysteamine Conjugates as Novel and Potent Autophagy Activators That Enhance the Correction of Misfolded F508del-Cystic Fibrosis Transmembrane Conductance Regulator (CFTR). J. Med. Chem., 60:458-473). Briefly, for the FRT cells, baseline measurements were taken for ˜20 minutes. Forskolin (10 μM) was first added to the apical and basolateral sides and then cells were treated with potentiator, VX-770 (1 μM). Finally, inhibitor-172 (Inh-172, 20 μM) was added to inactivate CFTR. The 16hBEge-W1812X-3F2 clones were measured similarly apart from forskolin and VX-770 added to the cells at the same time. Measurements were taken at two-minute intervals. Gt was calculated by the reciprocal of the recorded Rt (Gt=1/Rt), after Rt was corrected for solution resistance (Rs) and plotted as conductance traces (FIGS. 28C and 30A). Calculated equivalent currents (Ieq) were obtained similar and as outlined in Michaels et al (Michaels et al., (2020) Antisense oligonucleotide-mediated correction of CFTR splicing improves chloride secretion in cystic fibrosis patient-derived bronchial epithelial cells. Nucleic Acids Res., 48:7454-7467). Ieq was calculated using Ohm's law (Ieq=Vt/Rt) and plotted as current traces (FIGS. 31A and 32A). To estimate average functional response trajectories during each test period, area under the curve measurements of forskolin and forskolin+VX-770 were calculated using a one-third trapezoidal rule for each test period using Excel. The average of two identically treated wells was calculated for each plate to obtain one biological replicate used in the final mean±SEM graphed.

Statistics: Statistical analyses were performed using GraphPad PRISM 9.2.0. The specific statistical test used in each experiment can be found in the figure legends.

### Example 11. Splice-Switching Antisense Oligonucleotides for the Treatment of Class I CFTR Mutations

Premature termination codons (PTCs) generated by frameshift and nonsense mutations result in the synthesis of a truncated protein and trigger nonsense-mediated mRNA decay (NMD), largely abolishing functional protein production (1). Due to these multifaceted complications, treatments for diseases caused by terminating variants have been elusive. One therapeutic strategy for PTC-causing disease that has had clinical success uses splice-switching antisense oligonucleotides (ASOs) to induce exon skipping to eliminate pathogenic variants and/or reframe the mRNA transcript (2). This approach requires the exon targeted for ASO-induced skipping to be a symmetrical exon, defined as an exon in the same reading frame register as its flanking exons. Thus, when the symmetrical exon is spliced out, or skipped, the open reading frame is preserved. Symmetrical exon skipping is a common natural form of alternative splicing that can generate protein isoforms with altered functions (3). In the case of disease-associated variants in these exons, this mechanism can be harnessed using ASOs to remove PTCs and thereby stabilize the mRNA and recover protein expression. This approach can be therapeutic if the protein that is produced from the ASO-induced spliced transcript is at least partially functional. This strategy is the basis of the FDA-approved splice-switching antisense oligonucleotides targeting PTCs in Duchenne muscular dystrophy (DMD) (2).

To establish targets for exon-skipping strategies, an understanding of the function of protein isoforms encoded by mRNAs that lack a specific exon is required. In principle, rapid functional target identification can be achieved by systematic screening of all possible symmetrical exon deletions within a gene. Additionally, as this approach increases protein expression, small molecule drugs that target the protein can be screened for further rescue of protein function, as a potential combination therapy. The utility of this screening approach for investigating treatment strategies for cystic fibrosis (CF) is demonstrated herein.

CF is an autosomal recessive disease caused by disruption of the cystic fibrosis transmembrane conductance regulator (CFTR) gene that affects more than 100,000 people worldwide. CFTR encodes a transmembrane anion channel, the loss of which results in thick mucus buildup in multiple organs, most severely affecting the lungs and pancreas (4). There are thousands of different CF-associated CFTR variants that cause varying degrees of disease severity, based on their effect on expression and resulting protein function (5). For some variants, there are effective protein-targeted therapeutics approved to treat people with CF (pwCF). These include potentiator drugs, like Ivacaftor (VX-770), which enhance channel gating, and corrector drugs, used in combination with potentiators, which restore processing and trafficking of CFTR to the cell surface (6-8). The most prevalent treatment, Trikafta, is a combination of VX-770 and the correctors VX-661 and VX-445 (elexacaftor, tezacafter, ivacaftor: ETI) (7). These small molecule therapeutics, collectively referred to as highly effective modulator therapeutics (HEMTs), are effective in treating CF caused by more common variants, such as CFTR-F508del. However, because these drugs target the CFTR protein, they are not effective in treating CF caused by pathogenic variants that preclude protein production, such as those that produce PTCs. These types of variants account for ˜11% of all CF-causing variants and ˜62% of variants that are not eligible for treatment with current HEMTs (9-11).

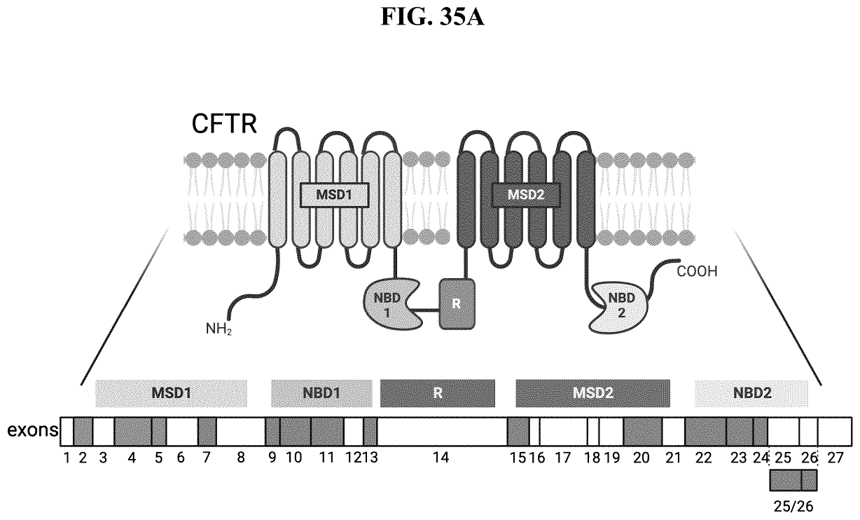

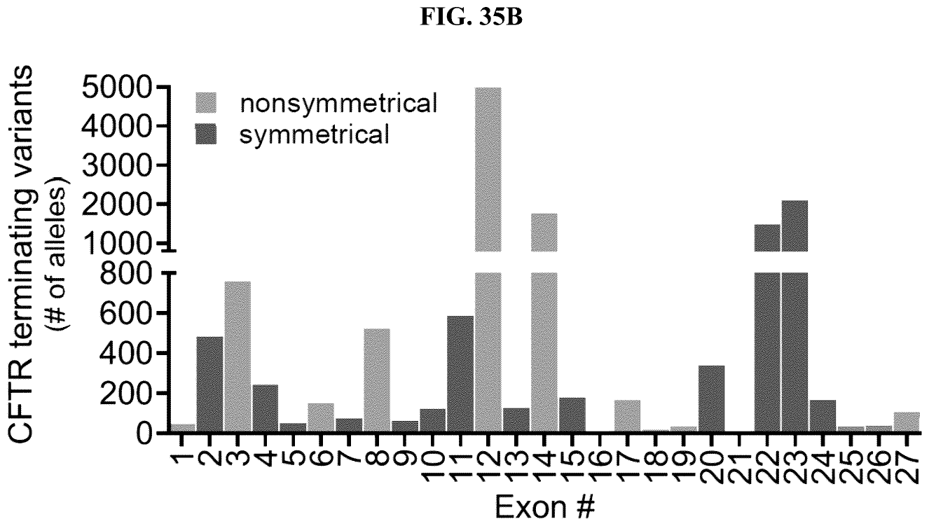

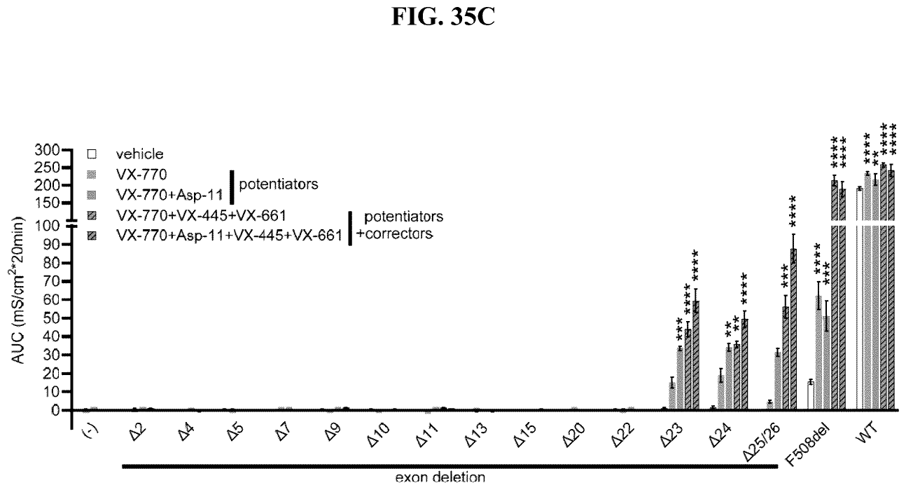

For one such variant, CFTR-W1282X, it has been reported that an effective ASO approach that corrects CFTR function by inducing the skipping of exon 23, a symmetrical exon which houses the variant (12-14). CFTR contains many other symmetrical exons that could be eliminated to remove variant-induced PTCs and NMD targets (FIG. 35A). If the protein isoform lacking an exon retains some function and/or is amenable to rescue by HEMTs, ASO-mediated skipping of these exons has the potential to treat any truncating variants that have been identified within these CFTR exons (FIG. 35B). As disclosed herein, utilizing a systematic engineered protein screening approach, identified CFTR symmetrical exons encoding amino acids in the C-terminus of the protein that can be removed and still produce a CFTR isoform with HEMT-responsive function. ASO-induced skipping of these exons can induce these isoforms, providing the beginnings of a therapeutic path forward for treating CF caused by variants in these exons. Overall, it is estimated that about 10% of CFTR variants that do not currently have effective treatment options may be responsive to this ASO-mediated exon skipping approach. These findings demonstrate the effectiveness of engineered protein screening to rapidly test for functional protein isoforms to guide strategic ASO design and assessment for exon skipping and functional protein production. This strategy offers a way to treat pathogenic terminating variants that can be broadly applied to any genetic disease.

### Materials and Methods

Generation of exon deletion expression plasmids: CFTR-Δexon deletion plasmids were constructed from the synthetic CFTR high codon adaption index (HCAI) into the pcDNA3.1/Neo (+) vector using the Q5 Site-Directed Mutagenesis Kit (NEB) (Table 12) (15). All plasmid deletions were sequenced. Plasmids were reverse transfected into Fischer Rat Thyroid (FRT) cells with lipofectamine LTX (Thermo Fisher) and OptiMEM (Thermo Fisher). Clonal cell lines were selected with G418 (300 μg/ml) and maintained in media supplemented with G418 (150 μg/ml).

TABLE 12
Primers flanking CFTR symmetrical exons used to
make CFTR exon deletion constructs.

| Primers | Sequence (5'-3') |
|---|---|
| 1: HCAI-CFTRdel2R | CTGAAGAACAGCTTGCTCACCAC |
| | (SEQ ID NO: 222) |
| 2: HCAI-CFTRdel2F | CGAGTGGGACCGCGAGCT |
| | (SEQ ID NO: 223) |
| 3: HCAI-CFTRdel4R | GCCCAGGTACAGGAAGATG |
| | (SEQ ID NO: 224) |
| 4: HCAI-CFTRdel4F | ACCCTGAAGCTGAGCAGC |
| | (SEQ ID NO: 225) |
| 5: HCAI-CFTRdel5R | CTTCTTGTAGATCAGGCTGAACA |
| | TGGCGATGC |
| | (SEQ ID NO: 226) |
| 6: HCAI-CFTRde15F | GGCCTGGCCCTGGCCCAC |
| | (SEQ ID NO: 227) |
| 7: HCAI-CFTRdel7R | CGGTACTTCATCATCATGC |
| | (SEQ ID NO: 228 |
| 8: HCAI-CFTRdel7F | GACCGAGCTGAAGCTGAC |
| | (SEQ ID NO: 229) |
| 9: HCAI-CFTRde19R | CTGGATCTTGTTGATGGCGC |
| | (SEQ ID NO: 230) |
| 10: HCAI-CFTRde19F | GGCTTCGGCGAGCTGTTC |
| | (SEQ ID NO: 231) |
| 11: HCAI-CFTRdel10R | CTCCTCCCAGAAGGCGGT |
| | (SEQ ID NO: 232) |
| 12: HCAI-CFTRdel10F | ACCAGCCTGCTGATGGTG |
| | (SEQ ID NO: 233) |
| 13: HCAI-CFTRdel11R | CTTGCCGGCGCCGGTGCT |
| | (SEQ ID NO: 234) |
| 14: HCAI-CFTRdel11F | GACATCAGCAAGTTCGCCGAGAA |
| | GGACAAC |
| | (SEQ ID NO: 235) |
| 15: HCAI-CFTRdel13R | CGGGCCAGGCTGATGCGG |
| | (SEQ ID NO: 236) |
| 16: HCAI-CFTRdel13F | CTGCGTGTGCAAGCTGATGGC |
| | (SEQ ID NO: 237) |
| 17: HCAI-CFTRdel15R | CTTCAGGTCCTCCTCGTTGATCTCC |
| | (SEQ ID NO: 238) |
| 18: HCAI-CFTRdel15F | GTGGCCGCCAGCCTGGTG |
| | (SEQ ID NO: 239) |
| 19: HCAI-CFTRdel20R | CCTCGCTCTCCAGCTGCTTCAGCTGC |
| | (SEQ ID NO: 240) |
| 20: HCAI-CFTRdel20F | GCGAGGGCGAGGGCCGCG |
| | (SEQ ID NO: 241) |
| 21: HCAI-CFTRdel22R | CAGGCTGTCCACGTCGATGCTGC |
| | (SEQ ID NO: 242) |
| 22: HCAI-CFTRdel22F | GTGGGCCTGCTGGGCCGC |
| | (SEQ ID NO: 243) |
| 23: HCAI-CFTRdel23R | GCGCTGGCCGGGGCTGAT |
| | (SEQ ID NO: 244) |
| 24: HCAI-CFTRdel23F | AAGGTGTTCATCTTCAGCGGCACCTTC |
| | (SEQ ID NO: 245) |
| 25: HCAI-CFTRdel24R | CTGGGGGATCACGCCGAAGG |
| | (SEQ ID NO: 246) |
| 26: HCAI-CFTRdel24F | GTGGGCCTGCGCAGCGTG |
| | (SEQ ID NO: 247) |
| 27: HCAI-CFTRdel2526R | GTGATCGAGGAGAACAAGGTGC |
| | (SEQ ID NO: 248) |
| 28: HCAI-CFTRdel2526F | CTCGTCGGCCACCTTCCA |
| | (SEQ ID NO: 249) |

Cells and culture conditions: FRT cell lines were cultured in F12 Coon's modification media (Sigma, F6636) supplemented with 10% fetal bovine serum (FBS) and 1% Penicillin-Streptomycin (PenStrep). T84 cells were cultured in Dulbecco's Modified Eagle Medium/Nutrient

Mixture F12 (DMEM/F12) media supplemented with 5% FBS and 1% PenStrep. Primary human bronchial epithelial (hBE) cells were acquired from the Compound Screening and Drug Discovery Core at RFUMS or the Cystic Fibrosis Foundation and thawed at passage (P) 1 or 2 for analysis. For functional studies cells were plated on 24-transwell filter plates (0.4 μM pore size, Polyester, Corning, catalog #CLS3397). FRT cells were grown in a liquid/liquid interface for 1 week. Primary cells were differentiated in an air/liquid interface for 5 weeks.

Antisense oligonucleotide treatment: Antisense oligonucleotides were 25-mer phosphorodiamidate morpholino oligomers (PMO) formulated in sterile water (Table 13). A non-targeting PMO was used as a negative control, ASO-C(Gene Tools LLC).

TABLE 13
Antisense oligonucleotide sequences designed
to skip symmetrical CFTR exons.

| ASOs | Sequence (5′-3′) | ASOs | Sequence (5′-3′) |
|---|---|---|---|
| 2-1 | ATCCTTTCCTCAAAATTGGTCTGGT | 13-2 | GGTATTCAAAGAACATACCTTTCAA |
| | (SEQ ID NO: 61) | | (SEQ ID NO: 93) |
| 2-2 | GTATATGTCTGACAATTCCAGGCGC | 15-1 | ACAATAGAACATTCTTACCTCTGCC |
| | (SEQ ID NO: 62) | | (SEQ ID NO: 94) |
| 2-3 | CAGATAGATTGTCAGCAGAATCAAC | 20-1 | CAAGATGAGTGAAAATTGGACTCCT |
| | (SEQ ID NO: 63) | | (SEQ ID NO: 110) |
| 2-4 | GTACATGAACATACCTTTCCAATTT | 20-2 | CGAAGGCACGAAGTGTCCATAGTCC |
| | (SEQ ID NO: 64) | | (SEQ ID NO: 111) |
| 4-1 | GAGGCTGTACTGCTTTGGTGACTTC | 20-3 | AACAGAGTTTCAAAGTAAGGCTGCC |
| | (SEQ ID NO: 65) | | (SEQ ID NO: 112) |
| 4-2 | GAAGCTATGATTCTTCCCAGTAAGA | 20-4 | AGTTGGCAGTATGTAAATTCAGAGC |
| | (SEQ ID NO: 66) | | (SEQ ID NO: 113) |
| 4-3 | GTGTAGGAGCAGTGTCCTCACAATA | 20-5 | TTCTATTCTCATTTGGAACCAGCGC |
| | (SEQ ID NO: 67) | | (SEQ ID NO: 114) |
| 4-4 | AATGTGATGAAGGCCAAAAATGGCT | 20-6 | GGTAACAGCAATGAAGAAGATGACA |
| | (SEQ ID NO: 68) | | (SEQ ID NO: 115) |
| 4-5 | GCTATTCTCATCTGCATTCCAATGT | 22-1 | ATGTCAATGAACTTAAAGACTCGGC |
| | (SEQ ID NO: 69) | | (SEQ ID NO: 116) |
| 4-6 | CCTGTGCAAGGAAGTATTACCTTCT | 22-2 | GGCCAGATGTCATCTTTCTTCACGT |
| | (SEQ ID NO: 70) | | (SEQ ID NO: 117) |
| 5-1 | CTAGAACACGGCTTGACAGCTTTAA | 22-3 | ATCTTTGACAGTCATTTGGCCCCCT |
| | (SEQ ID NO: 71) | | (SEQ ID NO: 118) |
| 5-2 | TGGAAAGGAGACTAACAAGTTGTCC | 22-4 | CCACCTTCTGTGTATTTTGCTGTGA |
| | (SEQ ID NO: 72) | | (SEQ ID NO: 119) |
| 7-1 | ACTGATCTTCCCAGCTCTCTGATCT | 22-5 | TCTCTAATATGGCATTTCCACCTTC |
| | (SEQ ID NO: 73) | | (SEQ ID NO: 120) |
| 7-2 | ATTTCTGAGGTAATCACAAGTCTTT | 22-6 | CCAGGACTTATTGAGAAGGAAATGT |
| | (SEQ ID NO: 74) | | (SEQ ID NO: 121) |
| 7-3 | AGTATGCCTTAACAGATTGGATATT | 22-7 | AAGCAGTGTTCAAATCTCACCCTCT |
| | (SEQ ID NO: 75) | | (SEQ ID NO: 122) |
| 7-4 | ATTTTTTCCATTGCTTCTTCCCAGC | 23-1 | ATCCAGTTCTTCCCAAGAGGCCCAC |
| | (SEQ ID NO: 76) | | (SEQ ID NO: 123) |
| 7-5 | ATTGGAACAACTTACTGTCTTAAGT | 23-2 | AGCTGATAACAAAGTACTCTTCCCT |
| | (SEQ ID NO: 77) | | (SEQ ID NO: 124) |
| 9-1 | TCCATCACTACTTCTGTAGTCGTTA | 23-B | AAGTTATTGAATCCCAAGACACACC |
| | (SEQ ID NO: 78) | | (SEQ ID NO: 125) |
| 9-2 | CTCCTCCCAGAAGGCTGTTACATTC | 23-A | CTAAGTCCTTTTGCTCACCTGTGGT |
| | (SEQ ID NO: 79) | | (SEQ ID NO: 126) |
| 9-3 | TTAAAAATTCTGACCTCCTCCCAGA | 24-1 | GATCACTCCACTGTTCATAGGGATC |
| | (SEQ ID NO: 80) | | (SEQ ID NO: 127) |
| 10-1 | GGCTGTCATCACCATTAGAAGTTTT | 24-2 | CTCATCTGCAACTTTCCATATTTCT |
| | (SEQ ID NO: 81) | | (SEQ ID NO: 128) |
| 10-2 | AATTACTGAAGAAGAGGCTGTCATC | 24-3 | ATTTCAGTTAGCAGCCTTACCTCAT |
| | (SEQ ID NO: 82) | | (SEQ ID NO: 129) |
| 10-3 | TAATATCTTTCAGGACAGGAGTACC | 25-1 | AGCCCAACCTGCAGTTAGAGAAGAT |
| | (SEQ ID NO: 83) | | (SEQ ID NO: 174) |
| 10-4 | GATCCAGCAACCGCCAACAACTGTC | 25-2 | TCTTCGCCTTACTGAGAACAGATCT |
| | (SEQ ID NO: 84) | | (SEQ ID NO: 175) |
| 10-5 | AGAACAAAAGAACTACCTTGCCTGC | 25-3 | AGAACATCTGAAACTCACACTGGAT |
| | (SEQ ID NO: 85) | | (SEQ ID NO: 176) |
| 11-1 | CTCCCATAATCACCATTAGAAGTGA | 25-EX | CTGGATCCAAATGAGCACTGGGTTA |
| | (SEQ ID NO: 86) | | (SEQ ID NO: 177) |
| 11-2 | ATTTTACCCTCTGAAGGCTCCAGTT | 26-1 | TACTGTGCAATCAGCAAATGCTTGT |
| | (SEQ ID NO: 87) | | (SEQ ID NO: 178) |
| 11-3 | ACAGAATGAAATTCTTCCACTGTGC | 26-2 | CCTGTGTTCACAGAGAATTACTGTG |
| | (SEQ ID NO: 88) | | (SEQ ID NO: 179) |
| 11-4 | GTGCCAGGCATAATCCAGGAAAACT | 26-3 | TCCAGCATTGCTTCTATCCTGTGTT |
| | (SEQ ID NO: 89) | | (SEQ ID NO: 180) |
| 11-5 | ATGCTTTGATGACGCTTCTGTATCT | 26-4 | GTTATAAAGACTCACCAAAAATTGT |
| | (SEQ ID NO: 90) | | (SEQ ID NO: 181) |
| 11-6 | TTTTCACATAGTTTCTTACCTCTTC | ASO-C | CCTCTTAGGTCAGTTACAATTTATA |
| | (SEQ ID NO: 91) | | (SEQ ID NO: 182) |
| 13-1 | TCTAGGTATCCAAAAGGAGAGTCTA | | |
| | (SEQ ID NO: 92) | | |

T84 cells were transfected with ASOs in DMEM/F12 media supplemented with 5% FBS and 1% PenStrep using Endo-Porter (Gene-Tools, 6 μl/ml) for 48 hours. Primary human bronchial epithelial (hBE) cells were transfected after differentiation on filter plates as previously described (12) using a 1-hour hypo-osmotic shock. The cells were maintained in Dulbecco's phosphate-buffered saline (DPBS) with ASO for 4 days until functional analysis.

RNA isolation, RT-PCR, and real-time qPCR: RNA was extracted from cells using TRIzol (Thermo Fisher Scientific). Reverse transcription was performed on total RNA using the GoScript Reverse Transcription System with an oligo-dT primer (Promega). Splicing was analyzed by radiolabeled PCR of resulting cDNA using GoTaq Green (Promega) supplemented with α-³²P-deoxycytidine triphosphate (dCTP). Primers for amplification are reported in Table 14. Reaction products were run on a 6% non-denaturing polyacrylamide gels and quantified using a Typhoon FLA 7000 phosphorimager (GE Healthcare) and ImageJ software.

Real-time qPCR was performed with PrimeTime Gene Expression Master Mix and PrimeTime qPCR probe assay kits for CFTR transcripts normalized to HPRT1 (IDT, Table 14). All reactions were analyzed in triplicate. Real-time PCR was performed on an Applied Biosystems (ABI) ViiA 7 Real-Time PCR System and analyzed by the ΔΔCT method.

TABLE 14
Primers and probes used to quantify mRNA splicing expression.

| Primers | Sequence (5′-3′) | Primers | Sequence (5′-3′) |
|---|---|---|---|
| 1: HCAI-CFTR- | ATGCAGCGCAGCCCCCTC | 25: hCFTR-ex10F | CTAATGGTGATGACAGCCTC |
| ex1F | (SEQ ID NO: 183) | | (SEQ ID NO: 206) |
| 2: HCAI-CFTR- | CGGTACTTCATCATCATGC | 26: hCFTR-ex12R | GCTCGTTGACCTCCACTCAG |
| ex6R | (SEQ ID NO: 254) | | (SEQ ID NO: 207) |
| 3: HCAI-CFTR- | ACCCTGAAGCTGAGCAGC | 27: hCFTR-ex12F | CACTGAGTGGAGGTCAACGA |
| ex5F | (SEQ ID NO: 184) | | (SEQ ID NO: 208) |
| 4: HCAI-CFTR- | CTCCTCCCAGAAGGCGGT | 28: hCFTR-ex14R | TCCAGGAGACAGGAGCATCT |
| ex9R | (SEQ ID NO: 185) | | (SEQ ID NO: 209) |
| 5: HCAI-CFTR- | GACCGAGCTGAAGCTGAC | 29: hCFTR-ex14F | AGGAGGCAGTCTGTCCTGAA |
| ex8F | (SEQ ID NO: 186) | | (SEQ ID NO: 210) |
| 6: HCAI-CFTR- | CTCCTCCAGCTGGCAGGC | 30: hCFTR-ex16R | CAGCACAACCAAAGAAGCAG |
| ex11R | (SEQ ID NO: 187) | | (SEQ ID NO: 211) |
| 7: HCAI-CFTR- | GACTTCCTGCAGAAGCAG | 31: hCFTR-ex19F | CAGTGCCAGTGATAGTG |
| ex9F | (SEQ ID NO: 188) | | (SEQ ID NO: 212) |
| 8: HCAI-CFTR- | CATCAGCTTGCACACGCA | 32: hCFTR-ex21R | CTCTTCCTTCTCCTTCTC |
| ex14RB | (SEQ ID NO: 189) | | (SEQ ID NO: 213) |
| 9: HCAI-CFTR- | GCAAAGTGAGCCTGGCCC | 33: hCFTR-ex21F | GGGCTGTAAACTCCAGCATA |
| ex14FE | (SEQ ID NO: 190) | | (SEQ ID NO: 214) |
| 10: HCAI-CFTR- | CCTCGCTCTCCAGCTGCTTCAGCTGC | 34: hCFTR-ex23R | GACACACCATCGATCTGG |
| ex19R | (SEQ ID NO: 191) | | (SEQ ID NO: 215) |
| 11: HCAI-CFTR- | GCGAGGGCGAGGGCCGCG | 35: hCFTR-ex22F | CCAAACCATACAAGAAT |
| ex21F | (SEQ ID NO: 192) | | (SEQ ID NO: 216) |
| 12: HCAI-CFTR- | ACGGGGTCCAGGTGGGCG | 36: hCFTR-ex24R | GATCACTCCACTGTTCAT |
| ex25R | (SEQ ID NO: 193) | | (SEQ ID NO: 217) |
| 13: hCFTR-ex1F | GCAGGCACCCAGAGTAGTAG | 37: hCFTR-ex23F | GGGAAGAGTACTTTGTTATC |
| | (SEQ ID NO: 194) | | (SEQ ID NO: 218) |
| 14: hCFTR-ex3R | GCCAGCTCTCTATCCCATTC | 38: hCFTR-ex25R | GTTCTATCACAGATCTGAG |
| | (SEQ ID NO: 195) | | (SEQ ID NO: 219) |
| 15: hCFTR-ex3F | GGGATAGAGAGCTGGCTTCA | 39: hCFTR-ex24F | CTTGGATCCCTATGAACA |
| | (SEQ ID NO: 196) | | (SEQ ID NO: 220) |
| 16: hCFTR-ex5R | TTGTTCAGGTTGTTGGAAAGG | 40: hCFTR-ex27R | CTAAAGCCTTGTATCTTG |
| | (SEQ ID NO: 197) | | (SEQ ID NO: 221) |
| 17: hCFTR-ex4F | CAAGGAGGAACGCTCTATCG | 41: hβ-actinFor | AAAGACCTGTACGCCAACAC |
| | (SEQ ID NO: 198) | | (SEQ ID NO: 156) |
| 18: hCFTR-ex6R | AGGCAGACGCCTGTAACAAC | 42: hβ-actinRev | GTCATACTCCTGCTTGCTGAT |
| | (SEQ ID NO: 199) | | (SEQ ID NO: 157) |
| 19: hCFTR-ex6F | ATGGGGCTAATCTGGGAGTT | 43: qhCFTR- | TGGCACCATTAAAGAAAATATCATCTT |
| | (SEQ ID NO: 200) | ex11WTF | (SEQ ID NO: 154) |
| 20: hCFTR-ex8R | CTGCCTTCCGAGTCAGTTTC | 44: qhCFTR- | CTCAGTGTGATTCCACCTTCTC |
| | (SEQ ID NO: 201) | ex12WTR | (SEQ ID NO: 155) |
| 21: hCFTR-ex8F | TCACCACCATCTCATTCTGC | 45: qhCFTR- | GGCACCATTAAAGAAAATATCATTGG |
| | (SEQ ID NO: 202) | ex11ΔFF | (SEQ ID NO: 168) |
| 22: hCFTR-ex10R | AAGAAGAGGCTGTCATCACCA | 46: qhCFTR- | CTCAGTGTGATTCCACCTTCT |
| | (SEQ ID NO: 203) | ex12ΔFR | (SEQ ID NO: 169) |
| 23: hCFTR-ex9F | TAACAGCCTTCTGGGAGGAG | 47: qhHPRT1For | GCGATGTCAATAGGACTCCAG |
| | (SEQ ID NO: 204) | | (SEQ ID NO: 170) |
| 24: hCFTR-ex11R | GGCATGCTTTGATGACGCTT | 48: qhHPRT1Rev | TTGTTGTAGGATATGCCCTTGA |
| | (SEQ ID NO: 205) | | (SEQ ID NO: 171) |

| | |
|---|---|
| hCFTR-DF508 | 56-FAM/ACAGAAGCG/ZEN/TCATCAAAGCATGCC/3IABKFQ (SEQ ID NO: 172) |
| hCFTR-F508 | 5.6-FAM/ACAGAAGCG/ZEN/TCATCAAAGCATGCC/3IABKFQ (SEQ ID NO: 158) |
| hHPRT1 | 5HEX/AGCCTAAGA/ZEN/TGAGAGTTCAAGTTGAGTTTGG/3IABKFQ (SEQ ID NO: 173) |

Protein isolation and immunoblot analysis: Cell lysates were prepared using NP-40 lysis buffer (1% Igepal. 150 mM NaCl. 50 mM Tris-HCl pH7.6) supplemented with 1× protease inhibitor cocktail (Sigma-Aldrich, cat #11836170001). Protein concentration was measured using a Coomassic (Bradford) protein assay (Thermo Fisher, cat #23200). Cell lysates were prepared with 4X NuPage LDS sample buffer (Invitrogen) for a final concentration of 1-2 mg/ml, incubated at 37° C. for 5 minutes, and loaded into a 4-12% Bis-Tris gel (Criterion) for SDS-PAGE separation in 1X MOPS buffer. Samples were transferred in 1X CAPS buffer to polyvinylidene difluoride membranes. Protein was detected using primary anti-CFTR antibodies 570 or 596 (Riordan lab UNC. Cystic Fibrosis Foundation, diluted 1:1000) and anti-β-actin (C4, Santa Cruz. Biotechnology, diluted 1:2000). Secondary anti-mouse poly-HRP was added for chemiluminescent detection with HRP substrate (CFTR: Femto.-actin: Classico; Immobulin. Sigma). Signal was quantified with ImageJ software.

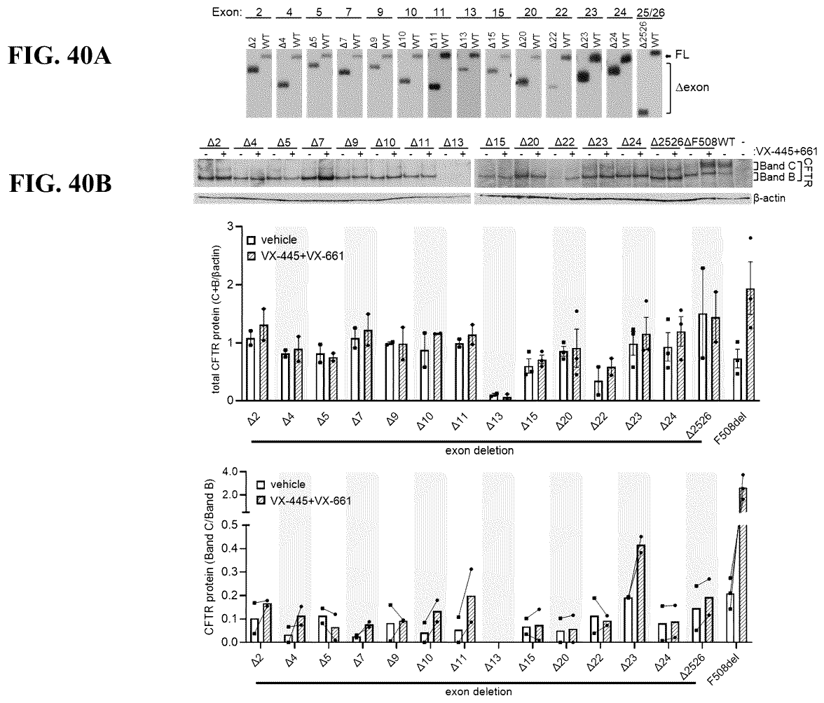

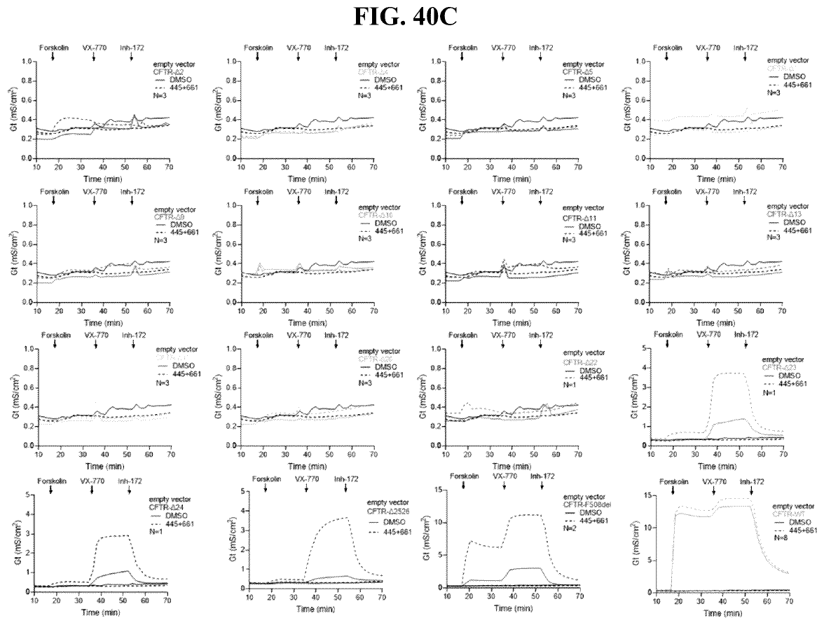

Automated conductance/equivalent current assay: FRT cells lines and primary hBE cells were treated with VX-445+VX-661 (1 μM+3 μM for hBEs or 3 μM+3.5 μM for FRTs. Selleckchem) or vehicle (0.1% or 0.2% DMSO) at 37° C. for 24 hours. Cells were switched from culture media to HEPES-buffered (pH 7.4) F12 Coon's modification running media (Sigma. F6636) apically and basolaterally and allowed to equilibrate for one hour at 37° C. without CO₂. For functional measurements the transepithelial voltage (Vt) and resistance (Rt) measurements were recorded at 37° C. with a 24-channel TECC robotic system (EP Design, Belgium) as previously described (12.16). Benzamil (6 μM), forskolin (10 μM). VX-770 (1 μM) with or without ASP-11 (20 μM), and Inh-172 (20 μM) or bumetanide (20 μM) additions were added sequentially. Conductance (Gt) was calculated by the reciprocal of the recorded Rt (Gt=1/Rt) and plotted as conductance traces (FIG. 40C). Equivalent currents from hBE donors were assessed similarly and calculated using Ohm's law (Ieq=Vt/Rt). Area under the curve measurements of forskolin, forskolin+VX-770, or forskolin+VX-770+ASP-11, were calculated using a one-third trapezoidal rule for each test period using Microsoft Excel.

Immunofluorescence Imaging: FRT cell lines grown on high precision glass 12 mm circle coverslips (No. 1.5H) (Paul Marlenfeld GmbH) were washed with PBS and fixed with 4% paraformaldehyde in PBS for 10 min at room temperature. Cells were permeabilized with 0.2% Triton X-100 in PBS for 5 min on ice. Subsequently, the cells were incubated in 3% bovine serum albumin for 20 minutes. Cells were incubated with the monoclonal anti-CFTR antibody (570, Riordan lab UNC. 1:500) and rabbit polyclonal anti-Claudin antibody (1:100. Zymed) for one hour. Cells were then incubated with goat anti-mouse secondary antibody conjugated with Alexa 488 and goat anti-rabbit secondary antibody conjugated with Alexa 594 (Invitrogen. 1:1000) for 1 hour and mounted using ProLong Gold antifade mounting reagent with DAPI (Life Technologies). Cells were observed on Zeiss Elyra structured illumination microscope (SIM) using a 63×1.40 NA objective using 7.-90 nm sectioning. In the axial direction, channel alignment was performed using 100 nm tetraspeck beads. Final images were prepared using Zeiss Zen software.

Statistics: Statistical analyses were performed using GraphPad PRISM 10.2.3 or Microsoft Excel. Specific statistical tests used in each experiment are reported in the figure legends.

### Results

### Systematic Deletion of CFTR Symmetrical Exons Reveals Functional Protein Isoforms

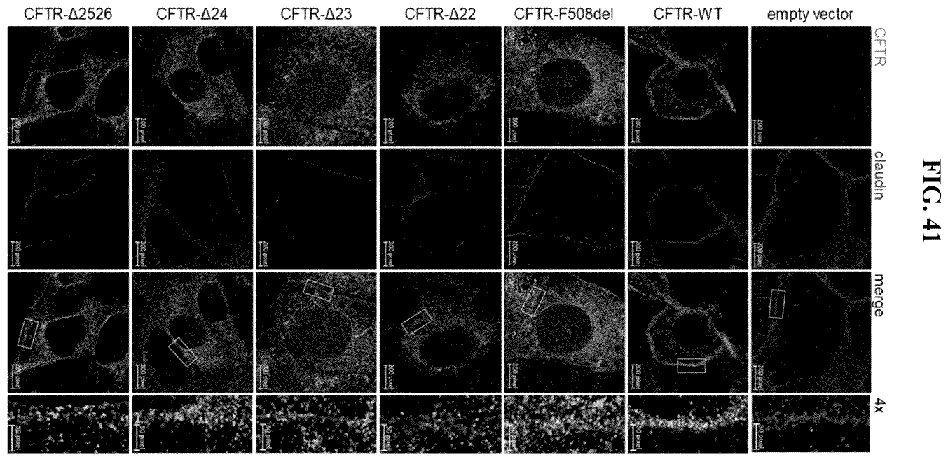

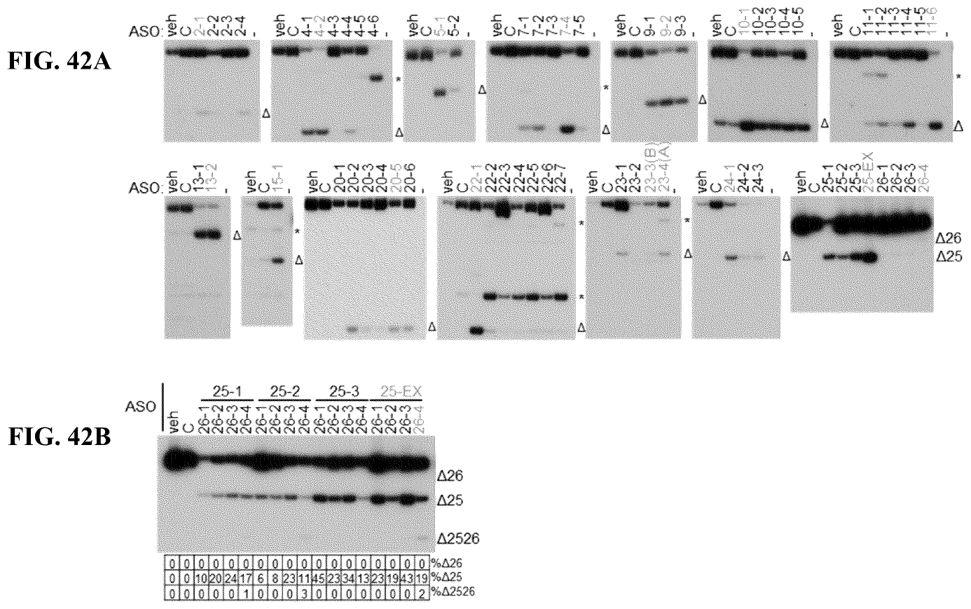

CFTR has 13 symmetrical exons that can be removed without disrupting the protein open reading frame. Additionally, the reading frame is maintained when exons 25 and 26 are eliminated together (FIG. 35A). It has been previously shown that ASO-induced skipping of exon 23 results in a partially functional CFTR protein when co-treated with established HEMTs (12-14). To identify additional pathogenic variants that could potentially be treated using this therapeutic approach, CFTR protein lacking each of the other symmetrical exons was analyzed. A series of CFTR plasmids with individual deletions of each of the 13 exons and both exons 25 and 26 together (CFTR-Δex) was created and analyzed CFTR expression and function after stable transfection into Fischer Rat Thyroid (FRT) cells, which do not express endogenous CFTR. CFTR-Δex isoform expression was confirmed by RT-PCR (FIG. 42).

FRT CFTR-Δex cell lines were grown to confluence on trans-well filter plates to assess the protein isoforms cAMP-activated conductance capabilities across the plasma membrane via forskolin stimulation. Responsiveness to HEMTs was also screened, including ETI and the novel potentiator Asp-11, which has been shown to work in synergy with VX-770 to further increase the function of CFTR protein isoforms, especially those that disrupt the second nucleic binding domain (NBD2) of CFTR (17,18). None of the expressed CFTR isoforms had measurable conductance in the FRT cells in the absence of HEMTs (white bars, FIG. 35C and FIG. 40). Treatment with VX-770 alone (grey bars, FIG. 35C) did not result in measurable conductance, however, co-potentiation with Asp-11 in combination with VX-770 (blue bars, FIG. 35C) resulted in significant forskolin-induced conductance from CFTR-423 and CFTR-A24 compared to vehicle-treated cells. Cells expressing CFTR-Δ23. CFTR-A24, and CFTR-425/26 had significant conductance following ETI treatment (grey hashed bars, FIG. 35C) and all three deletion isoforms responded most robustly when treated with a combination of Asp-11 and ETI (blue hashed bars, FIG. 35C).

Conductance activity did not correlate with expression as immunoblot analysis of the C-terminal deletion protein isoforms did not show significant expression differences above the other deletion isoforms (FIG. 42). Preliminary analysis of protein processing showed an increase in mature CFTR Band C protein after ETI treatment in comparison to the immature Band B, indicative of recovery of proper CFTR folding and trafficking. These results provide evidence that this activity was specific to modulator effects on the isoforms (FIG. 42). Additionally, immunofluorescence analysis of cells expressing CFTR-Δex23, 24 and 25/26 showed appropriate localization of the expressed proteins to the cell membrane after ETI treatment (FIG. 41). Taken together, these findings indicate that CFTR isoforms lacking exons 23, 24, or 25 and 26 together can transport chloride across the membrane when treated with HEMTs, making them good candidates for combined HEMT and ASO-mediated exon skipping therapies.

### Splice-Switching ASOs Induce Skipping of CFTR Symmetrical Exons

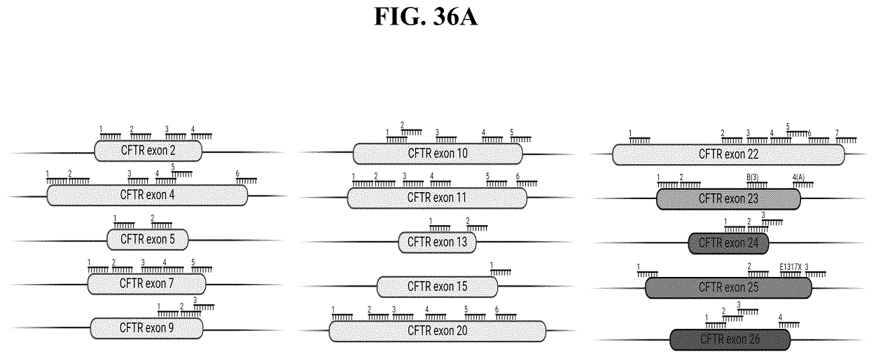

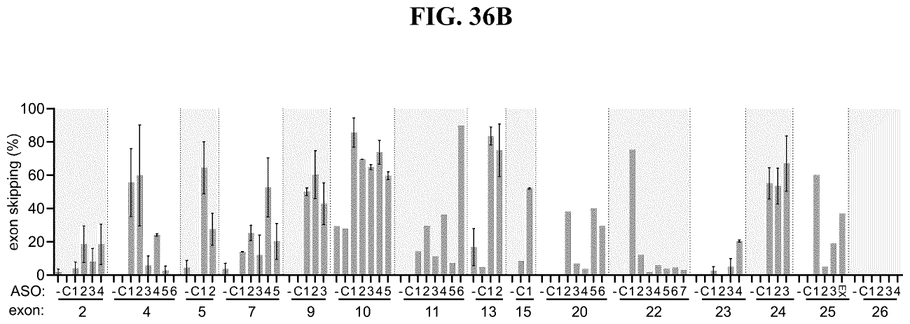

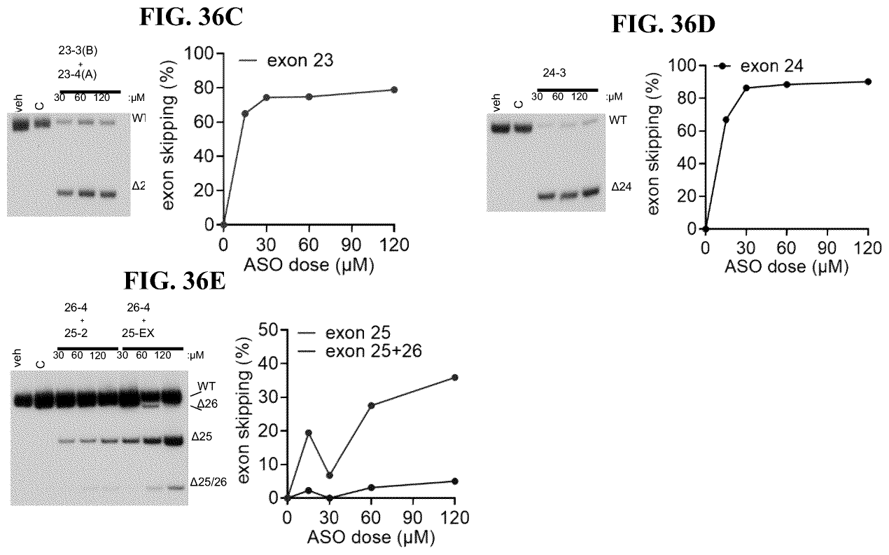

Next splice-switching ASOs that target each symmetrical exon for skipping were tested. A series of ASOs for each exon were designed to base-pair with the canonical acceptor and donor splice sites or putative exonic splicing enhancer sequences and further selected for their predicted binding affinity (FIG. 36A). ASOs were tested for their ability to induce exon skipping of endogenous CFTR-WT pre-mRNA by transfection into T84 cells. RT-PCR analysis of RNA isolated after 48 hours of incubation identified at least one ASO that was effective at skipping each target exon, except exon 26 where combination with exon 25 skipping ASOs was necessary (FIG. 36B, FIG. 42).

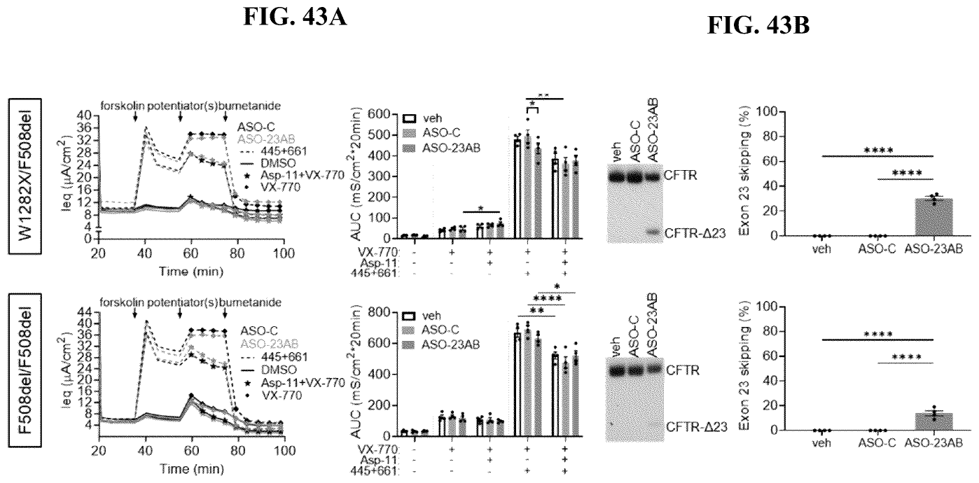

This functional screen indicated that only isoforms lacking exon 23, 24 or 25/26 are responsive to HEMT and thus, focus was put on ASOs targeting these exons. An ASO that base-pairs to the 5′ splice site region of exon 23 (ASO-23A) induces skipping of exon 23, but also causes splicing to a cryptic splice site that results in an out-of-frame CFTR mRNA. Additional treatment with an ASO that base-pairs at the cryptic splice site (ASO-23B) eliminates cryptic splicing and greatly enhances skipping of exon 23 in a dose-dependent manner (FIG. 36C) (12). Likewise, a robust dose-dependent increase in exon 24 was observed following treatment with a single ASO, ASO-24-3 (FIG. 36D). Treatment with ASO-25-2 or 25-EX, which targets the CFTR-E1371X variant, alone or in combination with ASO-26-4 (ASO-25EX+26) resulted in the skipping of exon 25 or 26 individually or both exons 25 and 26 together (Δ25/26) (FIG. 36E, FIG. 43). Though dual skipping of exon 25 and 26 was not induced at a high level, higher dosing of ASO-25EX+26 provided some improvement (FIG. 36E). Because elimination of exon 25 alone would lead to PTCs in the penultimate exon 26, which may escape NMD (1), ASO treatment could provide sufficient splice modulation to increase CFTR mRNA and protein for further rescue with HEMTs.

Functional CFTR Isoforms Generated by ASO-Induced Symmetrical Exon Skipping in cells derived from CF patient airways

It was recently reported that treatment with ASOs that induce exon 23 skipping can rescue CFTR function in cells homozygous for CFTR-W1282X (12). This rescue required treatment with ETI. This disclosure tested whether compound heterozygous cells expressing only a single copy of CFTR-W1282X along with another variant that is associated with minimally functional CFTR could produce sufficient CFTR-Δex23 to provide a functional benefit in genotypes that aren't currently approved for HEMTs. Co-potentiation with Asp-11 was also tested, to assess whether greater functional rescue could be achieved by modulator optimization. CFTR function was assessed via equivalent current analysis in human bronchial epithelial cells (hBE) isolated from pwCF.

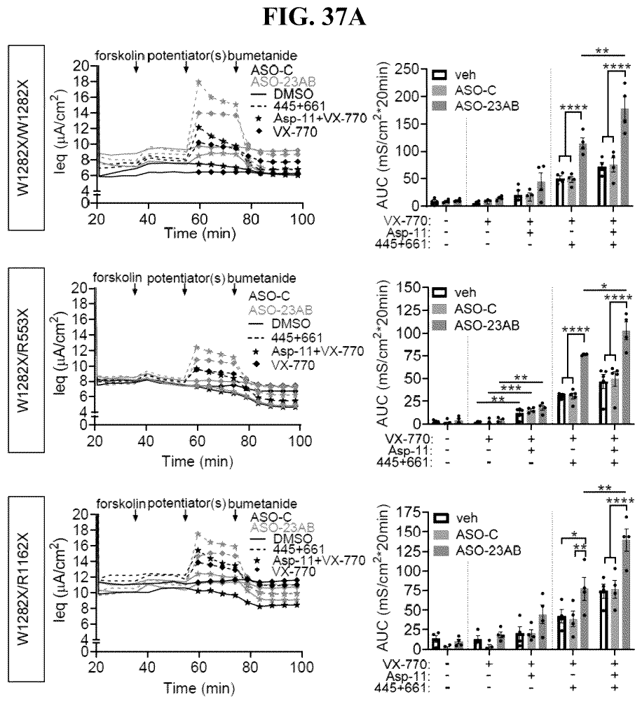

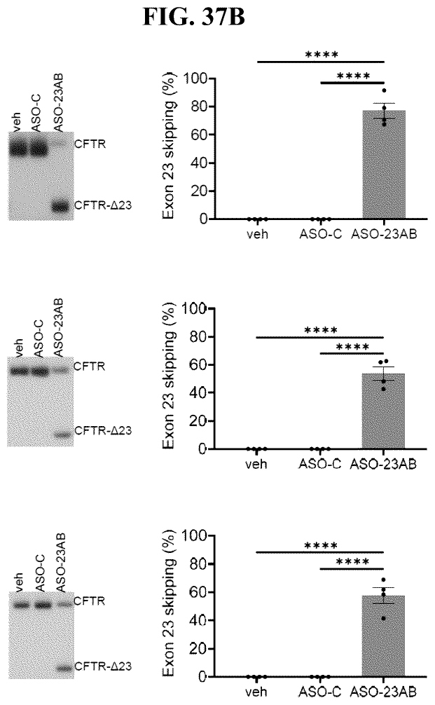

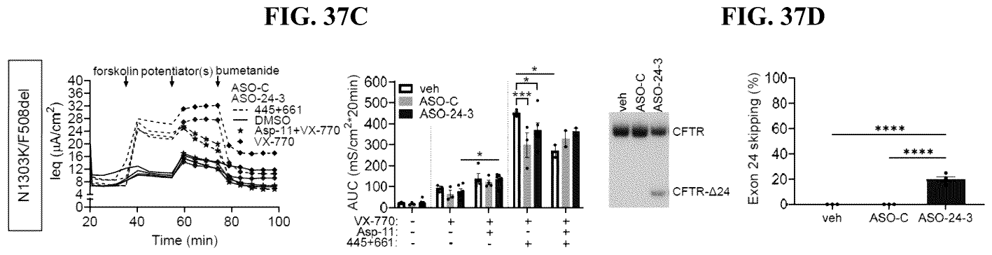

Cells from donors expressing two copies of CFTR-W1282X or with one copy and another nonsense variant, CFTR-R553X or CFTR-R1162X, were treated with vehicle, ASO-C, or ASO-23AB with or without Asp-11 and ETI. In all donors, treatment with ASO-23AB resulted in a significant increase in chloride currents over the control ASO when treated with ETI, and co-treatment with Asp-11 resulted in an even greater ASO-induced rescue of CFTR function (FIG. 37A). In donor cells with CFTR-W1282X and CFTR-F508del, ASO-23AB treatment had little effect on the rescue already achieved by ETI alone, similar to previous results (12). Interestingly in these cells, ASO-23AB and co-potentiator treatment with VX-770 and Asp-11 together resulted in significant rescue of CFTR function, but Asp-11 addition with ETI lowered functional recovery (FIG. 43). This effect on ETI-dependent function was also observed in cells homozygous for CFTR-F508del, where ASO-23AB treatment had no effect on modulator rescue (FIG. 43). Co-potentiation alone provided a significant rescue in cells from the CFTR-W1282X/CFTR-R553X donor (FIG. 37A). These results suggest that HEMT optimization could both provide significant rescue for minimal function variants and further improve the ASO-induced CFTR-Δex23 isoform function. The results also caution that effects on the second CFTR allele should be considered, as differential modulator effects may occur.

It has been reported that ASO-induced exon skipping could have partial allele specificity for CFTR-W1282X due to the elimination of splicing factor binding sites (12). Similarly here, ASO treatment induced ˜80% exon 23 skipping in cells homozygous for CFTR-W1282X (FIG. 37B). In compound heterozygous cells, ASO treatment induced ˜30-60% exon skipping (FIG. 37B, FIG. 43). In cells homozygous for CFTR-F508del, ASO-23AB treatment induced less than 20% skipping of the wild-type exon 23 (FIG. 43), providing strong evidence that exon 23 containing the W1282X sequence is more susceptible to ASO-23AB-induced skipping than the wild-type sequence. These results suggest that ASO-induced exon 23 skipping could be therapeutic for pwCF expressing at least one copy of CFTR-W1282X and not eligible for, or responsive to, current therapeutics.

Skipping of exon 24 also results in a CFTR protein that is functional when treated with HEMTs (FIG. 35). Thus, the effect of ASO-induced exon 24 skipping on CFTR function in patient donor cells was also evaluated. Unfortunately, airway cells from a donor with a terminating variant in exon 24 were not available. Instead, ASO-24-3 was tested in hBE cells derived from a person with the CFTR-N1303K variant, a CF-associated missense variant in exon 24 that results in a protein with defective biogenesis and gating abnormalities. This variant has shown some response to HEMTs but is not currently FDA-approved for HEMT treatment (17,19,20). Given the activity of the CFTR-A24 isoform (FIG. 35), it was hypothesized that CFTR-424 would have greater activity than CFTR-N1303K and, because of this enhanced activity, ASO-induced skipping of exon 24 would result in elevated currents in the donor cells. Compound heterozygote hBE cells expressing CFTR-N1303K and CFTR-F508del were transfected with ASO-24-3 and treated with ETI and Asp-11. Compared to the control ASO, there was little effect of ASO-24-3 treatment on ETI rescue of CFTR activity and Asp-11 had no additional effect (FIG. 37C). This result suggests that ASO-induced skipping of exon 24 will not provide a therapeutic benefit over ETI to individuals with CFTR-N1303K and CFTR-F508del. However, ASO-24-3 and co-potentiator treatment did result in a significant rescue over VX-770 alone (FIG. 37C) despite inducing only modest (˜20%) exon 24 skipping (FIG. 37D), suggesting that further optimization of ASO-induced skipping could result in greater activity, particularly in the context of other CF variants that are not responsive to ETI. Nonetheless, CFTR-N1303K is an imperfect model for exon skipping therapies as there is no evidence of mRNA degradation. Further testing in donor cells expressing variants in exon 24 that destabilize CFTR mRNA is needed to assess the efficacy of ASO-induced exon 24 skipping.

### A Potential Functional CFTR Isoform Generated by ASO-Induced Multiexon Skipping

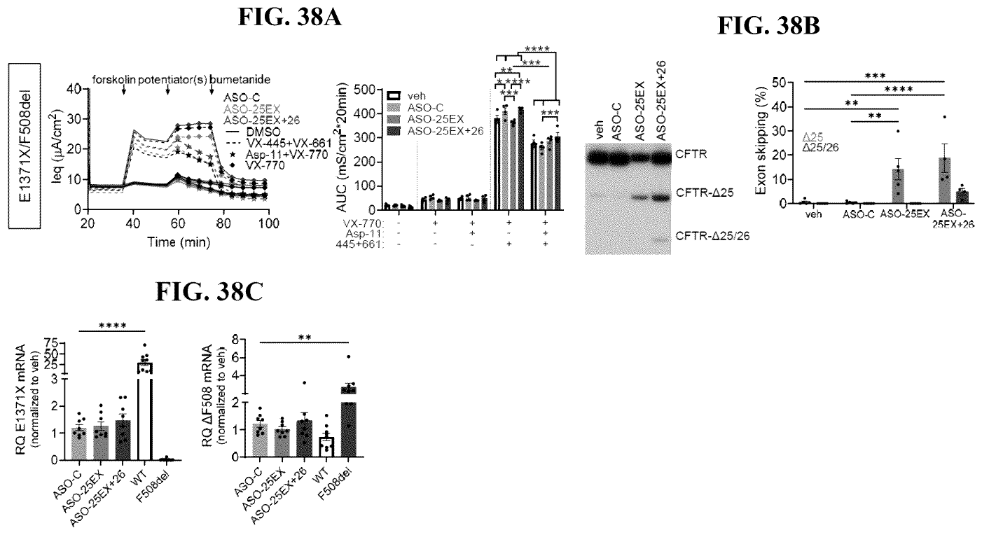

Similar to CFTR-D23 and CFTR-D24. CFTR-D25/26 retains conductance when treated with ETI and is responsive to co-potentiation with Asp-11 (FIG. 35C). ASO-25-EX alone induces robust exon 25 skipping (FIG. 36B), which could be therapeutic if the downstream PTC in the penultimate exon does not activate NMD. The combination of ASO-26-4 with ASO-25EX (ASO-25EX+26) results in modest skipping of both exons 25 and 26 (FIG. 36E). To test these ASOs further, hBE cells that are compound heterozygous for CFTR-E1371X, a PTC in exon 25, and CFTR-F508del, were treated with ASO-25-EX alone or ASO-25EX+26 and assessed for CFTR activity in the presence or absence of ETI and Asp-11. ASO treatment had little effect on activity without ETI treatment (FIG. 38A). With ETI. ASO-25EX+26 showed no additional rescue and ASO-25EX resulted in slightly lower function compared to the control ASO. As seen before in cells expressing CFTR-F508del. Asp-11 treatment reduced ETI response but did show a significant increase in function in combination with ASO-25EX+26 treatment compared to control treated cells, which may suggest that Asp-11 has some specificity for CFTR-425/26, which could lead to elevated function in genotypes heterozygous with variants other than CFTR-F508del (FIG. 38A). ASO-25EX treatment resulted in ˜10-20% exon 25 skipping alone or in combination with ASO-26-4 and ASO-25EX+26 induced a low level of skipping of both exons 25 and 26 (FIG. 38C). Allele-specific qPCR analysis revealed a trend of increased CFTR-E1371X mRNA expression with ASO-25-EX+26 treatment despite the minimal skipping, suggesting that ASO treatment may effectively stabilize mRNA produced from CFTR-E1371X, presumably due to the removal of the PTC in the ASO-induced CFTR25/26 isoform (FIG. 38C). The upward trend in mRNA levels, and the modest effect on CFTR function with ETI and Asp-11 treatment suggests that further optimization of ASO-induced exon 25 and 26 skipping could be beneficial to people with terminating variants in exons 25 or 26.

Inactive CFTR Produced from ASO-Induced Exon 22 Skipping Confirms Functional Limits of Exon-Deleted Isoforms

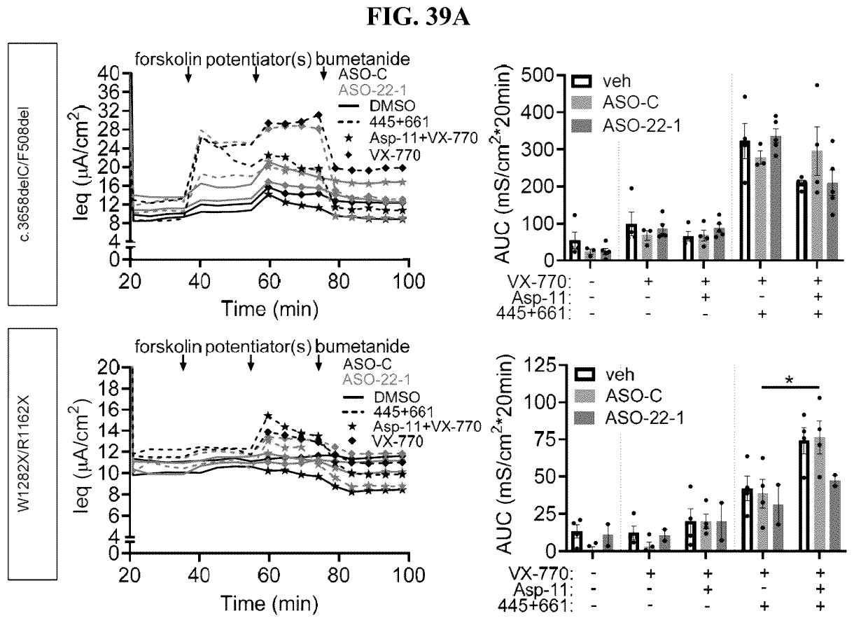

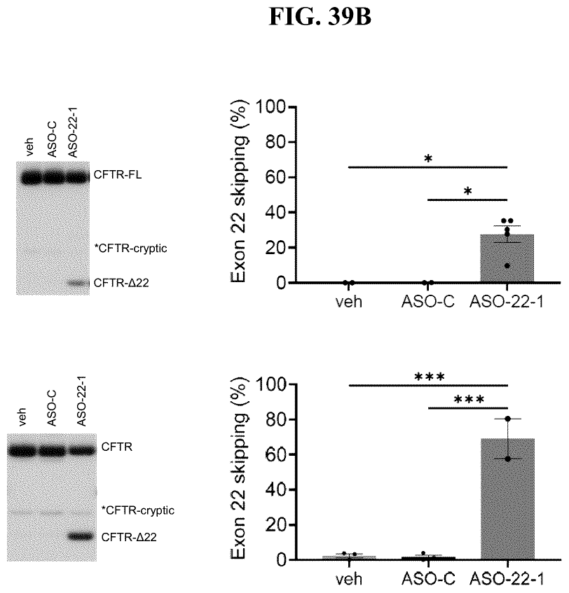

The systematic screening of protein isoforms engineered by ASO-induced exon skipping revealed that CFTR C-terminal isoforms lacking amino acids encoded by exons 23, 24, or 25 and 26, but not exon 22 or any other upstream exon, are functional as chloride channels when treated with ETI (FIG. 35). To confirm the results found in this functional screen, ASO-induced skipping of exon 22 using ASO-22-1 was tested in hBE cells derived from two pwCF expressing CFTR-R1162X (compound heterozygous with W1282X) or CFTR-c.3658delC (p.K1177SfsX15) (compound heterozygous with F508del). In the CFTR-c.3658delC cells, ASO-22-1 treatment did not improve activity relative to that already achieved by ETI treatment alone and Asp-11 had no additional effects (FIG. 39A). In the CFTR-R1162X cells, ETI and Asp-11 treatment significantly improved CFTR function in control treated cells, likely due to the effect on CFTR-W1282X, but treatment with ASO-22-1 eliminated this response (FIG. 39A). Lack of functional recovery was not due to insufficient skipping of exon 22 as treatment with ASO-22-1 resulted in ˜30% exon 22 skipping in CFTR-3659delC cells and ˜70% in CFTR-R1162X cells (FIG. 39B). These results, along with the conductance activity measured from CFTR-422 in FRT cells, suggest that ASO-induced skipping of exon 22 does not result in an active CFTR isoform and that the protein domains encoded by exons 1-22, are likely essential for functional activity and rescue with HEMTs. These results define the boundaries of any potential exon-skipping therapy for CF as sequences downstream of exon 22 and suggest that truncation or deletions after CFTR exon 22 could provide a therapeutic benefit for all variants effecting expression in CFTR exons 23-27.

### DISCUSSION

Identifying targets of ASO-mediated exon skipping that can restore functional potential to an inactivated protein is relevant to therapeutic development for any disease caused by pathogenic variants that result in PTCs and destabilize the mRNA and/or truncate the protein, including CF. To this end, this study demonstrates the systematic testing of each so-called skippable (i.e. symmetrical) exon of the CFTR gene for its functional dispensability and identifies four exons at the 3′ end of the transcript as viable targets for restoring function by ASO-induced exon skipping.

The potential of ASO-induced exon skipping for treating CF caused by CFTR-W1282X has been previously demonstrated (12-14). Additional testing of this approach in patient cells expressing genotypes not currently eligible for HEMTs provides strong evidence that combining ASO treatment with HEMTs will provide therapeutic benefit to these patients and others that have terminating variants in exon 23 (FIG. 37). The testing of additional exon deletion isoforms in comparison to the CFTR-Δex23 protein product, demonstrates that CFTR-Δex24 and CFTR-Δex25/26 have comparable levels of function and HEMT response (FIG. 35), providing evidence that this strategy could be beneficial to terminating variants in additional C-terminal exons. The subsequent screening identifies ASOs that induce skipping of these exons (FIG. 36).

Further ASO design and testing in CF patient cells is needed to advance these approaches. However, screening is limited by a lack of patient cells with required genotypes needed for functional testing, limiting our analysis to cells with less-than-ideal CFTR genotypes for definitively evaluating therapeutic benefit. As such, no additional benefit over ETI was observed with ASO-mediated exon 24 skipping in patient cells expressing CFTR-N1303K with CFTR-F508del (FIG. 37). However, because CFTR-N1303K has not been shown to compromise CFTR expression, it is a sub-optimal target for this strategy. Furthermore, the tested donor cells co-expression with CFTR-F508del makes it difficult to assess ASO+HEMT efficacy against the CFTR-F508del ETI response. Significant ASO-specific rescue was seen with co-potentiation treatment alone, suggesting modulated CFTR-424 has some benefit. However, further testing in patients with terminating variants in exon 24 and other undruggable genotypes is needed. Regarding exon 25/26 skipping, responses from patient cells expressing CFTR-E1371X revealed a significant response from ASO-25EX+26 compared to ASO-C treated cells with Asp-11 addition to ETI. However, again, ASO-specific functional rescue of CFTR-E1371X directly was difficult to assess with CFTR-F508del. These results, as well as a trend of increased expression of CFTR-E1371X RNA despite low levels of exon 25/26 skipping, suggest that ASO treatment could be promising for terminating variants in these exons with further optimization (FIG. 38).

In addition to ASOs, a number of alternative therapeutic strategies are being explored to improve stability and translation of mRNA with terminating mutations, though currently none have been successfully implemented in patients. Current strategies involve approaches to block NMD and/or increase translational read-through of PTCs to recover full-length protein expression (21-27). One downfall of such candidate therapeutics is that, global dysregulation of these processes could be detrimental if specificity for the PTC-containing transcript is not achieved. ASOs offer some advantages for design and development that present the potential for a relatively short development timeline, which is especially critical to individuals with these variants that currently have no good treatment options, including a large population of pwCF.

ASOs may offer some advantage over other strategies as a validated therapeutic platform, proven largely safe in humans. In-frame exon skipping strategies utilizing ASOs have been successfully applied to address PTCs in DMD (2) and explored in other disease models (36-42). ASOs also offer a potential advantage over larger therapeutic cargo in delivery to target tissues. ASO delivery to the respiratory system, one of the primary targets for CF therapeutics, has been demonstrated clinically for asthma and other inflammatory lung conditions (43) and most recently as an antiviral approach for Sars-CoV-2 (44,45). Naked ASOs have been successfully delivered to the lung and enter multiple cell types including CFTR expressing epithelial cells (46-49), though questions about whether the targeted cell-types are relevant for CF treatment remain (50). ASOs have long-lasting effects in primary hBE cells isolated from pwCF (16), and could have similar durability in vivo considering ASO activity for other indications can last weeks to months in terminally differentiated cells (51,52). Lung epithelia may turnover relatively slowly, with estimates of 6-18 months (53). Aerosolization of ASOs as a delivery modality has been demonstrated and targeted CFTR transcripts in both a CF-like lung disease model in mice and monkeys as well as pwCF (48,49,54,55). However, ASOs for CF have not yet advanced beyond clinical trials. Additionally, people with terminating variants tend to have severe disease phenotypes in other organs, so systemic delivery of ASOs must also be considered (56). Intravenous and subcutaneous ASO injections are currently used to treat other diseases, demonstrating the potential for systemic delivery of ASOs for CF (57-59). Successful ASO delivery to the affected organs in pwCF and target engagement with CFTR will be essential to therapeutic success.

In addition to therapeutic discovery, exploring the effects of exon deletions can provide information on protein function that can inform and facilitate future drug discovery and development. The systematic screening of CFTR exon deletion isoforms as disclosed herein indicates that the C-terminal exons are not essential for the fundamental function of CFTR when HEMTs are applied, a result confirmed by ASO testing in CF airway cells. Previous studies have shown that proteins with C-terminal truncations up to amino acid 1217 retain partial surface expression and function and are responsive to HEMTs (60-63). The domains encoded by exons 23, 24, and 25/26 encompass parts of NBD2 (NBD2=1203-1440, 423=41240-1291, 424=41292-1321, and Δ2526=A1321-1413), and the retained function of these exon deletions, but not exons upstream, is consistent with these reports (FIG. 35C). CFTR-Δ23 and CFTR-424 also retain critical sites in NBD2 for ATP-binding and NBD dimerization (64), which are eliminated in CFTR-425/26. Similar conductance levels between the isoforms (FIG. 35C) suggest that, if this region is critical for dimerization, NBD dimerization is not critical for proper CFTR function with HEMT treatment. Furthermore, removal of PTCs in these exons allows for translation to the C-terminus, which includes amino acids important for stability, gating, and localization (63,65-67). Overall, the functional data shows that elimination of exon 23, 24, or 25/26 results in CFTR protein responsive to HEMT rescue and suggests that ASO-mediated exon skipping of CFTR after exon 22 could be a therapeutic pathway. In addition, these results provide evidence that pwCF with splicing mutations that eliminate exons 23-26 are good candidates for ETI treatment if not currently approved for its use.

Co-potentiation with Asp-11, previously shown to increase the activity of other variants that are unresponsive to ETI, further enhanced CFTR isoform function (FIG. 35-39) (17,18). This result suggests that new modulator discovery focused on enhancing function from CFTR deletion isoforms could provide additional benefit to ASO-induced rescue of expression, especially considering that the binding sites of the modulators are eliminated by some deletions. For example, the deletion of exon 20 eliminates a potential VX-770 binding pocket and, though not responsive to VX-770, might be responsive to alternative potentiators (69). Class I correctors bind in the first transmembrane domain (TMD1) and could be affected by several of the deletions, including exon 9 which has been previously explored for exon skipping and showed no response to the class 1 corrector VX-809 (70,71). The differing binding sites of VX-445 and VX-661 improve the efficacy of ETI and similar approaches to modulator design may be beneficial for each deletion construct (72).

Taken together, these results not only inform CF drug development, but also have important implications for treating other diseases caused by terminating variants. Treating such variants by eliminating the exons they reside in and increasing productive protein expression could be a broadly applied therapeutic strategy. This investigation into the effects of symmetrical exon removal on CFTR expression and function provides a roadmap for the systematic evaluation of symmetrical exon deletion, revealing additional ASO therapeutic targets for CF drug development and instructing on a therapeutic strategy for discovering treatment approaches for other diseases.

### REFERENCES

1. Das, R. and Panigrahi, G. K. (2024) Messenger RNA Surveillance: Current Understanding, Regulatory Mechanisms, and Future Implications. Mol Biotechnol.
2. Gupta, S., Sharma, S. N., Kundu, J., Pattanayak, S. and Sinha, S. (2023) Morpholino oligonucleotide-mediated exon skipping for DMD treatment: Past insights, present challenges and future perspectives. J Biosci, 48.
3. Magen, A. and Ast, G. (2005) The importance of being divisible by three in alternative splicing. Nucleic Acids Res, 33, 5574-5582.
4. Cheng, S. H., Gregory, R.J., Marshall, J., Paul, S., Souza, D. W., White, G. A., O'Riordan, C. R. and Smith, A. E. (1990) Defective intracellular transport and processing of CFTR is the molecular basis of most cystic fibrosis. Cell, 63, 827-834.
5. Guo, J., Garratt, A. and Hill, A. (2022) Worldwide rates of diagnosis and effective treatment for cystic fibrosis. J Cyst Fibros, 21, 456-462.
6. Van Goor, F., Hadida, S., Grootenhuis, P. D. J., Burton, B., Stack, J. H., Straley, K. S., Decker, C. J., Miller, M., McCartney, J., Olson, E. R. et al. (2011) Correction of the F508del-CFTR protein processing defect in vitro by the investigational drug VX-809. Proceedings of the National Academy of Sciences of the United States of America, 108, 18843-18848.
7. Ferreira, F. C., Buarque, C. D. and Lopes-Pacheco, M. (2024) Organic Synthesis and Current Understanding of the Mechanisms of CFTR Modulator Drugs Ivacaftor, Tezacaftor, and Elexacaftor. Molecules, 29.
8. Van Goor, F., Hadida, S., Grootenhuis, P. D. J., Burton, B., Cao, D., Neuberger, T., Turnbull, A., Singh, A., Joubran, J., Hazlewood, A. et al. (2009) Rescue of CF airway epithelial cell function in vitro by a CFTR potentiator, VX-770. Proceedings of the National Academy of Sciences, 106, 18825-18830.
9. Bear, C. E. (2020) A Therapy for Most with Cystic Fibrosis. Cell, 180, 211-211.
10. De Boeck, K., Zolin, A., Cuppens, H., Olesen, H. V. and Viviani, L. (2014) The relative frequency of CFTR mutation classes in European patients with cystic fibrosis. Journal of cystic fibrosis: official journal of the European Cystic Fibrosis Society, 13, 403-409.
11. Cutting, G. R., Castellani, C., Corey, M., Lewis, M. H., Penland, C. M., Raraigh, K. S., Rommens, J. M. and Sosnay, P.R. (2013), Cftr2.
12. Michaels, W. E., Pena-Rasgado, C., Kotaria, R., Bridges, R. J. and Hastings, M. L. (2022) Open reading frame correction using splice-switching antisense oligonucleotides for the treatment of cystic fibrosis. Proc Natl Acad Sci USA, 119, e2114886119.
13. Kim, Y. J., Sivetz, N., Layne, J., Voss, D. M., Yang, L., Zhang, Q. and Krainer, A. R. (2022) Exon-skipping antisense oligonucleotides for cystic fibrosis therapy. Proc Natl Acad Sci U S A, 119.
14. Oren, Y. S., Avizur-Barchad, O., Ozeri-Galai, E., Elgrabli, R., Schirelman, M. R., Blinder, T., Stampfer, C. D., Ordan, M., Laselva, O., Cohen-Cymberknoh, M. et al. (2022) Antisense oligonucleotide splicing modulation as a novel Cystic Fibrosis therapeutic approach for the W1282X nonsense mutation. J Cyst Fibros, 21, 630-636.
15. Shah, K., Cheng, Y., Hahn, B., Bridges, R., Bradbury, N. A. and Mueller, D. M. (2015) Synonymous Codon Usage Affects the Expression of Wild Type and F508del CFTR. Journal of Molecular Biology, 427, 1464-1479.
16. Michaels, W. E., Bridges, R. J. and Hastings, M. L. (2020) Antisense oligonucleotide-mediated correction of CFTR splicing improves chloride secretion in cystic fibrosis patient-derived bronchial epithelial cells. Nucleic Acids Res, 48, 7454-7467.
17. Phuan, P. W., Tan, J. A., Rivera, A. A., Zlock, L., Nielson, D. W., Finkbeiner, W. E., Haggie, P. M. and Verkman, A.S. (2019) Nanomolar-potency ‘co-potentiator’ therapy for cystic fibrosis caused by a defined subset of minimal function CFTR mutants. Sci Rep, 9, 17640.
18. Phuan, P. W., Son, J. H., Tan, J. A., Li, C., Musante, I., Zlock, L., Nielson, D. W., Finkbeiner, W. E., Kurth, M. J., Galietta, L. J. et al. (2018) Combination potentiator (‘co-potentiator’) therapy for CF caused by CFTR mutants, including N1303K, that are poorly responsive to single potentiators. J Cyst Fibros, 17, 595-606.
19. Sadras, I., Kerem, E., Livnat, G., Sarouk, I., Breuer, O., Reiter, J., Gileles-Hillel, A., Inbar, O., Cohen, M., Gamliel, A. et al. (2023) Clinical and functional efficacy of elexacaftor/tezacaftor/ivacaftor in people with cystic fibrosis carrying the N1303K mutation. J Cyst Fibros, 22, 1062-1069.
20. Destefano, S., Gees, M. and Hwang, T.-C. (2018) Physiological and pharmacological characterization of the N1303K mutant CFTR. Journal of Cystic Fibrosis, 17, 573-581.
21. Venturini, A., Borrelli, A., Musante, I., Scudieri, P., Capurro, V., Renda, M., Pedemonte, N. and Galietta, L. J. V. (2021) Comprehensive Analysis of Combinatorial Pharmacological Treatments to Correct Nonsense Mutations in the CFTR Gene. Int J Mol Sci, 22.
22. Ko, W., Porter, J. J., Sipple, M. T., Edwards, K. M. and Lueck, J.D. (2022) Efficient suppression of endogenous CFTR nonsense mutations using anticodon-engineered transfer RNAs. Mol Ther Nucleic Acids, 28, 685-701.
23. Albers, S., Allen, E. C., Bharti, N., Davyt, M., Joshi, D., Perez-Garcia, C.G., Santos, L., Mukthavaram, R., Delgado-Toscano, M. A., Molina, B. et al. (2023) Engineered tRNAs suppress nonsense mutations in cells and in vivo. Nature, 618, 842-848.
24. Carollo, P.S., Tutone, M., Culletta, G., Fiduccia, I., Corrao, F., Pibiri, I., Di Leonardo, A., Zizzo, M. G., Melfi, R., Pace, A. et al. (2023) Investigating the Inhibition of FTSJ1, a Tryptophan tRNA-Specific 2′-O-Methyltransferase by NV TRIDs, as a Mechanism of Readthrough in Nonsense Mutated CFTR. Int J Mol Sci, 24.
25. Leubitz, A., Vanhoutte, F., Hu, M.-Y., Porter, K., Gordon, E., Tencer, K., Campbell, K., Banks, K. and Haverty, T. (2021) A Randomized, Double-Blind, Placebo-Controlled, Multiple Dose Escalation Study to Evaluate the Safety and Pharmacokinetics of ELX-02 in Healthy Subjects. Clinical pharmacology in drug development, 10, 859-869.
26. Temaj, G., Telkoparan-Akillilar, P., Nuhii, N., Chichiarelli, S., Saha, S. and Saso, L. (2023) Recoding of Nonsense Mutation as a Pharmacological Strategy. Biomedicines, 11.
27. Keenan, M. M., Huang, L., Jordan, N. J., Wong, E., Cheng, Y., Valley, H. C., Mahiou, J., Liang, F., Bihler, H., Mense, M. et al. (2019) Nonsense-mediated RNA Decay Pathway Inhibition Restores Expression and Function of W1282X CFTR. Am J Respir Cell Mol Biol, 61, 290-300.
28. Kim, Y. J., Nomakuchi, T., Papaleonidopoulou, F., Yang, L., Zhang, Q. and Krainer, A. R. (2022) Gene-specific nonsense-mediated mRNA decay targeting for cystic fibrosis therapy. Nat Commun, 13, 2978.
29. Nomakuchi, T. T., Rigo, F., Aznarez, I. and Krainer, A. R. (2016) Antisense oligonucleotide-directed inhibition of nonsense-mediated mRNA decay. Nature Biotechnology, 34, 164-166.
30. Lomunova, M. A. and Gershovich, P.M. (2023) Gene Therapy for Cystic Fibrosis: Recent Advances and Future Prospects. Acta Naturae, 15, 20-31.
31. Li, C., Liu, Z., Anderson, J., Liu, Z., Tang, L., Li, Y., Peng, N., Chen, J., Liu, X., Fu, L. et al. (2023) Prime editing-mediated correction of the CFTR W1282X mutation in iPSCs and derived airway epithelial cells. PLOS One, 18, e0295009.
32. Mention, K., Cavusoglu-Doran, K., Joynt, A. T., Santos, L., Sanz, D., Eastman, A. C., Merlo, C., Langfelder-Schwind, E., Scallan, M. F., Farinha, C. M. et al. (2023) Use of adenine base editing and homology-independent targeted integration strategies to correct the cystic fibrosis causing variant, W1282X. Hum Mol Genet, 32, 3237-3248.
33. Belbellaa, B., Reutenauer, L., Messaddeq, N., Monassier, L. and Puccio, H. (2020) High Levels of Frataxin Overexpression Lead to Mitochondrial and Cardiac Toxicity in Mouse Models. Mol Ther Methods Clin Dev, 19, 120-138.
34. Rowe, S. M., Zuckerman, J. B., Dorgan, D., Lascano, J., McCoy, K., Jain, M., Schechter, M. S., Lommatzsch, S., Indihar, V., Lechtzin, N. et al. (2023) Inhaled mRNA therapy for treatment of cystic fibrosis: Interim results of a randomized, double-blind, placebo-controlled phase ½ clinical study. J Cyst Fibros, 22, 656-664.
35. Vaidyanathan, S., Kerschner, J. L., Paranjapye, A., Sinha, V., Lin, B., Bedrosian, T. A., Thrasher, A. J., Turchiano, G., Harris, A. and Porteus, M. H. (2024) Investigating adverse genomic and regulatory changes caused by replacement of the full-length CFTR cDNA using Cas9 and AAV. Mol Ther Nucleic Acids, 35, 102134.
36. Molinari, E., Ramsbottom, S.A., Srivastava, S., Booth, P., Alkanderi, S., McLafferty, S. M., Devlin, L. A., White, K., Gunay-Aygun, M., Miles, C. G. et al. (2019) Targeted exon skipping rescues ciliary protein composition defects in Joubert syndrome patient fibroblasts. Sci Rep, 9, 10828.
37. Leier, A., Moore, M., Liu, H., Daniel, M., Hyde, A. M., Messiaen, L., Korf, B. R., Selvakumaran, J., Ciszewski, L., Lambert, L. et al. (2022) Targeted exon skipping of NF1 exon 17 as a therapeutic for neurofibromatosis type I. Mol Ther Nucleic Acids, 28, 261-278. Ablinger, M., Lettner, T., Friedl, N., Potocki, H., Palmetzhofer, T., Koller, U., Illmer, J., 38. Liemberger, B., Hainzl, S., Klausegger, A. et al. (2021) Personalized Development of Antisense Oligonucleotides for Exon Skipping Restores Type XVII Collagen Expression in Junctional Epidermolysis Bullosa. International Journal of Molecular Sciences, 22, 3326.
39. Lee, J. J. A., Maruyama, R., Duddy, W., Sakurai, H. and Yokota, T. (2018) Identification of Novel Antisense-Mediated Exon Skipping Targets in DYSF for Therapeutic Treatment of Dysferlinopathy. Molecular Therapy-Nucleic Acids, 13, 596-604.
40. Yamamura, T., Horinouchi, T., Adachi, T., Terakawa, M., Takaoka, Y., Omachi, K., Takasato, M., Takaishi, K., Shoji, T., Onishi, Y. et al. (2020) Development of an exon skipping therapy for X-linked Alport syndrome with truncating variants in COL4A5. Nature Communications, 11.
41. Centa, J. L., Stratton, M. P., Pratt, M. A., Osterlund Oltmanns, J. R., Wallace, D. G., Miller, S. A., Weimer, J. M. and Hastings, M. L. (2023) Protracted CLN3 Batten disease in mice that genetically model an exon-skipping therapeutic approach. Mol Ther Nucleic Acids, 33, 15-27.
42. Rodriguez-Polo, I. and Behr, R. (2022) Exploring the Potential of Symmetric Exon Deletion to Treat Non-Ischemic Dilated Cardiomyopathy by Removing Frameshift Mutations in TTN. Genes (Basel), 13.
43. Liao, W., Dong, J., Peh, H. Y., Tan, L. H., Lim, K. S., Li, L. and Wong, W.F. (2017) Oligonucleotide Therapy for Obstructive and Restrictive Respiratory Diseases. Molecules, 22.
44. Zhu, C., Lee, J. Y., Woo, J. Z., Xu, L., Nguyenla, X., Yamashiro, L. H., Ji, F., Biering, S. B., Van Dis, E., Gonzalez, F. et al. (2022) An intranasal ASO therapeutic targeting SARS-COV-2. Nat Commun, 13, 4503.
45. Yu, A. M. and Tu, M. J. (2022) Deliver the promise: RNAs as a new class of molecular entities for therapy and vaccination. Pharmacol Ther, 230, 107967.
46. Brinks, V., Lipinska, K., de Jager, M., Beumer, W., Button, B., Livraghi-Butrico, A., Henig, N. and Matthee, B. (2019) The Cystic Fibrosis-Like Airway Surface Layer Is not a Significant Barrier for Delivery of Eluforsen to Airway Epithelial Cells. J Aerosol Med Pulm Drug Deliv, 32, 303-316.
47. Crosby, J. R., Zhao, C., Jiang, C., Bai, D., Katz, M., Greenlee, S., Kawabe, H., McCaleb, M., Rotin, D., Guo, S. et al. (2017) Inhaled ENaC antisense oligonucleotide ameliorates cystic fibrosis-like lung disease in mice. J Cyst Fibros, 16, 671-680.
48. Zhao, C., Crosby, J., Lv, T., Bai, D., Monia, B. P. and Guo, S. (2019) Antisense oligonucleotide targeting of mRNAs encoding ENaC subunits alpha, beta, and gamma improves cystic fibrosis-like disease in mice. J Cyst Fibros, 18, 334-341.
49. Ozeri-Galai, E., Friedman, L., Barchad-Avitzur, O., Markovetz, M. R., Boone, W., Rouillard, K. R., Stampfer, C. D., Oren, Y. S., Hill, D. B., Kerem, B. et al. (2023) Delivery Characterization of SPL84 Inhaled Antisense Oligonucleotide Drug for 3849+10 kb C->T Cystic Fibrosis Patients. Nucleic Acid Ther, 33, 306-318.
50. Shin, M., Chan, I. L., Cao, Y., Gruntman, A. M., Lee, J., Sousa, J., Rodriguez, T. C., Echeverria, D., Devi, G., Debacker, A. J. et al. (2022) Intratracheally administered LNA gapmer antisense oligonucleotides induce robust gene silencing in mouse lung fibroblasts. Nucleic Acids Res, 50, 8418-8430.
51. Luu, K. T., Norris, D. A., Gunawan, R., Henry, S., Geary, R, and Wang, Y. (2017) Population Pharmacokinetics of Nusinersen in the Cerebral Spinal Fluid and Plasma of Pediatric Patients With Spinal Muscular Atrophy Following Intrathecal Administrations. J Clin Pharmacol, 57, 1031-1041.
52. Migliorati, J. M., Liu, S., Liu, A., Gogate, A., Nair, S., Bahal, R., Rasmussen, T. P., Manautou, J. E, and Zhong, X. B. (2022) Absorption, Distribution, Metabolism, and Excretion of US Food and Drug Administration-Approved Antisense Oligonucleotide Drugs. Drug Metab Dispos, 50, 888-897.
53. Rawlins, E. L. and Hogan, B. L. (2008) Ciliated epithelial cell lifespan in the mouse trachea and lung. Am J Physiol Lung Cell Mol Physiol, 295, L231-234.
54. Drevinek, P., Pressler, T., Cipolli, M., De Boeck, K., Schwarz, C., Bouisset, F., Boff, M., Henig, N., Paquette-Lamontagne, N., Montgomery, S. et al. (2020) Antisense oligonucleotide eluforsen is safe and improves respiratory symptoms in F508DEL cystic fibrosis. J Cyst Fibros, 19, 99-107.
55. Fajac, I. and De Boeck, K. (2017) New horizons for cystic fibrosis treatment. Pharmacol Ther, 170, 205-211.
56. Orenti, A., Pranke, I., Faucon, C., Varilh, J., Hatton, A., Golec, A., Dehillotte, C., Durieu, I., Reix, P., Burgel, P. R. et al. (2023) Nonsense mutations accelerate lung disease and decrease survival of cystic fibrosis children. J Cyst Fibros, 22, 1070-1079.
57. Ardizzone, S., Bevivino, G. and Monteleone, G. (2016) Mongersen, an oral Smad7 antisense oligonucleotide, in patients with active Crohn's disease. Therap Adv Gastroenterol, 9, 527-532.
58. Quemener, A. M., Centomo, M. L., Sax, S. L. and Panella, R. (2022) Small Drugs, Huge Impact: The Extraordinary Impact of Antisense Oligonucleotides in Research and Drug Development. Molecules, 27.
59. Sheng, L., Rigo, F., Bennett, C. F., Krainer, A. R. and Hua, Y. (2020) Comparison of the efficacy of MOE and PMO modifications of systemic antisense oligonucleotides in a severe SMA mouse model. Nucleic Acids Res, 48, 2853-2865.
60. Cui, L., Aleksandrov, L., Chang, X. B., Hou, Y. X., He, L., Hegedus, T., Gentzsch, M., Aleksandrov, A., Balch, W. E, and Riordan, J. R. (2007) Domain Interdependence in the Biosynthetic Assembly of CFTR. Journal of Molecular Biology, 365, 981-994.
61. Kai, D. and Lukacs, G. L. (2009) Cooperative assembly and misfolding of CFTR domains in vivo. Mol Biol Cell, 20, 1903-1915.
62. Haggie, P. M., Phuan, P. W., Tan, J. A., Xu, H., Avramescu, R. G., Perdomo, D., Zlock, L., Nielson, D. W., Finkbeiner, W. E., Lukacs, G. L. et al. (2017) Correctors and potentiators rescue function of the truncated W1282X-Cystic Fibrosis Transmembrane Regulator (CFTR) translation product. Journal of Biological Chemistry, 292, 771-785.
63. Ostedgaard, L. S., Randak, C., Rokhlina, T., Karp, P., Vermeer, D., Ashbourne Excoffon, K. J. and Welsh, M. J. (2003) Effects of C-terminal deletions on cystic fibrosis transmembrane conductance regulator function in cystic fibrosis airway epithelia. Proc Natl Acad Sci USA, 100, 1937-1942.
64. Stratford, F. L., Ramjeesingh, M., Cheung, J. C., Huan, L. J. and Bear, C. E. (2007) The Walker B motif of the second nucleotide-binding domain (NBD2) of CFTR plays a key role in ATPase activity by the NBD1-NBD2 heterodimer. Biochem J, 401, 581-586.
65. Gentzsch, M. and Riordan, J. R. (2001) Localization of sequences within the C-terminal domain of the cystic fibrosis transmembrane conductance regulator which impact maturation and stability. Journal of Biological Chemistry, 276, 1291-1298.
66. Gentzsch, M., Aleksandrov, A., Aleksandrov, L, and Riordan, J. R. (2002) Functional analysis of the C-terminal boundary of the second nucleotide binding domain of the cystic fibrosis transmembrane conductance regulator and structural implications. The Biochemical journal, 366, 541-548.
67. Sharma, N., LaRusch, J., Sosnay, P. R., Gottschalk, L. B., Lopez, A. P., Pellicore, M. J., Evans, T., Davis, E., Atalar, M., Na, C. H. et al. (2016) A sequence upstream of canonical PDZ-binding motif within CFTR COOH-terminus enhances NHERFI interaction. Am J Physiol Lung Cell Mol Physiol, 311, L1170-L1182.
68. Micoud, J., Chauvet, S., Scheckenbach, K. E. L., Alfaidy, N., Chanson, M. and Benharouga, M. (2015) Involvement of the heterodimeric interface region of the nucleotide binding domain-2 (NBD2) in the CFTR quaternary structure and membrane stability. Biochimica et Biophysica Acta-Molecular Cell Research.
69. Laselva, O., Qureshi, Z., Zeng, Z. W., Petrotchenko, E. V., Ramjeesingh, M., Hamilton, C. M., Huan, L. J., Borchers, C. H., Pomes, R., Young, R. et al. (2021) Identification of binding sites for ivacaftor on the cystic fibrosis transmembrane conductance regulator. iScience, 24, 102542.
70. Fiedorczuk, K. and Chen, J. (2022) Mechanism of CFTR correction by type I folding correctors. Cell, 185, 158-168 e111.
71. Martinovich, K. M., Kicic, A., Stick, S. M., Johnsen, R. D., Fletcher, S. and Wilton, S. D. (2022) Investigating the Implications of CFTR Exon Skipping Using a Cftr Exon 9 Deleted Mouse Model. Front Pharmacol, 13, 868863.
72. Veit, G., Roldan, A., Hancock, M. A., Da Fonte, D.F., Xu, H., Hussein, M., Frenkiel, S., Matouk, E., Velkov, T. and Lukacs, G. L. (2020) Allosteric folding correction of F508del and rare CFTR mutants by elexacaftor-tezacaftor-ivacaftor (Trikafta) combination. JCI Insight, 5.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A composition comprising one or more modified oligonucleotides, wherein each of the one or more modified oligonucleotides consists of 8 to 30 linked nucleosides, wherein the nucleobase sequence of each of the one or more modified oligonucleotides is at least 80% complementary to an equal-length portion of a target region of a cystic fibrosis transmembrane conductance regulator (CFTR) transcript, wherein the target region is within:
(a) nucleobase 198,888 and nucleobase 199,094 of SEQ ID NO: 130;
(b) nucleobase 199,705 and nucleobase 199,796 of SEQ ID NO: 130.

2. The composition of claim 1, wherein:
(a) the nucleobase sequence of each of the one or more modified oligonucleotides is at least 80% complementary to an equal-length portion within nucleobase 198,888 and nucleobase 199,094 of SEQ ID NO: 130; or
(b) the nucleobase sequence of each of the one or more modified oligonucleotides is at least 80% complementary to an equal-length portion within nucleobase 199,705 and nucleobase 199,796 of SEQ ID NO: 130.

3. The composition of claim 1, wherein:
(a) the target region is within nucleobase 198,888 and nucleobase 199,094 of SEQ ID NO: 130, and each of the one or more modified oligonucleotides is selected from the group consisting of SEQ ID NOs: 174-177; or
(b) the target region is within nucleobase 199,705 and nucleobase 199,796 of SEQ ID NO: 130, and each of the one or more modified oligonucleotides is selected from the group consisting of SEQ ID NOs: 178-181.

4. The composition of claim 1, wherein the composition comprises a modified oligonucleotide selected from SEQ ID NO: 174-177, and a modified oligonucleotide selected from SEQ ID NO: 178-181.

5. The composition of claim 1, wherein the nucleobase sequence of each of the one or more modified oligonucleotides is at least 85%, at least 90%, at least 95%, at least 96%, at least 97%, at least 98%, at least 99%, or at least 100% complementary to an equal-length portion of the target region.

6. The composition of claim 1, wherein each of the one or more modified oligonucleotides comprises at least one modified nucleoside selected from a modified sugar moiety, a 2′-substituted sugar moiety, a 2′OME, a 2′F, a 2′-MOE, a bicyclic sugar moiety, a LNA, a cEt, a sugar surrogate, a morpholino, or a modified morpholino.

7. The compound of claim 1, wherein each of the one or more modified oligonucleotides comprises at least 5, at least 10, at least 15, at least 20 or at least 25 modified nucleosides, each independently comprising a modified sugar moiety.

8. The composition of claim 1, wherein each nucleoside of each of the one or more modified oligonucleotides is a modified nucleoside, each independently comprising a modified sugar moiety.

9. The composition of claim 1, wherein each of the one or more modified oligonucleotides comprises at least two modified nucleosides comprising modified sugar moieties that are the same as one another or that are different from one another.

10. The composition of claim 1, wherein each of the one or more modified oligonucleotides comprises a modified region of at least 5, at least 10, at least 15, at least 16, at least 17, at least 18, at least 19, or at least 20 contiguous modified nucleosides.

11. The composition of claim 10, wherein each modified nucleoside of the modified region has a modified sugar moiety independently selected from: 2′-F, 2′-OMe, 2′-MOE, cEt, LNA, morpholino, and modified morpholino.

12. The composition of claim 10, wherein the modified nucleosides of the modified region each comprise the same modification as one another.

13. The composition of claim 10, wherein the modified nucleosides of the modified region each comprise the same 2′-substituted sugar moiety selected from: 2′-F, 2′-OMe, and 2′-MOE.

14. The composition of claim 10, wherein the modified nucleosides of the modified region each comprise the same bicyclic sugar moiety selected from: LNA and cEt.

15. The composition of claim 10, wherein the modified nucleosides of the modified region each comprises a sugar surrogate, and wherein the sugar surrogate of the modified nucleosides of the region of modified nucleosides is a morpholino.

16. The composition of claim 1, wherein each of the one or more modified oligonucleotides comprises at least one modified internucleoside linkage.

17. The composition of claim 16, wherein the at least one modified internucleoside linkage comprises at least one phosphorothioate internucleoside linkage.

18. The composition of claim 16, wherein each internucleoside linkage is a modified internucleoside linkage and wherein each internucleoside linkage comprises the same modification.

19. The composition of claim 16, wherein each internucleoside linkage is a phosphorothioate internucleoside linkage.

20. The composition of claim 1, comprising at least one conjugate.

21. The composition of claim 1, wherein the composition modulates splicing or expression of the CFTR transcript.

22. The composition of claim 1, further comprising one or more Cystic fibrosis transmembrane conductance regulator (CFTR) modulators.

23. The composition of claim 22, wherein the one or more CFTR modulators are selected from ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof.

24. A pharmaceutical composition comprising at least one composition according to claim 1 and a pharmaceutically acceptable carrier or diluent.

25. A method of modulating splicing or expression of a CFTR transcript in a cell comprising contacting the cell with at least one composition according to claim 1.

26. The method of claim 25, wherein the cell is in vitro or in vivo.

27. A method of treating cystic fibrosis, comprising administering at least one composition according to claim 1 to an animal in need thereof.

28. The method of claim 27, wherein the administering step comprises delivering to the animal by inhalation, parenteral injection or infusion, oral, subcutaneous or intramuscular injection, buccal, transdermal, transmucosal, and topical.

29. The method of claim 27, wherein the animal is a human or a mouse.

30. The method of claim 27, further comprising administering one or more Cystic fibrosis transmembrane conductance regulator (CFTR) modulators.

31. The method of claim 30, wherein the one or more CFTR modulators are selected from ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof.

32. A method of treating cystic fibrosis, comprising administering the pharmaceutical composition of claim 24 to an animal in need thereof.

33. The method of claim 32, further comprising administering one or more Cystic fibrosis transmembrane conductance regulator (CFTR) modulators.

34. The method of claim 33, wherein the one or more CFTR modulators are selected from ivacaftor (VX-770), lumacaftor (VX-809), tezacaftor (VX-661), elexacaftor (VX-445), bamocaftor (VX-659), olacaftor (VX-440), vanzacaftor (VX-121), deutivacaftor (VX-561) (formerly CTP-656), VX-152, ABBV-2222 (galicaftor, formerly GLPG2222), ABBV-3221, ABBV-3067, ABBV-191, ABBV-974 (formerly GLPG-1837), ABBV-2451 (formerly GLPG-2451), ABBV-3067 (formerly GLPG3067), ELX-02 (NB124), FDL169, cavonstat (N91115), MRT5005, ataluren (PTC124), posencaftor (PTI-801), nesolicaftor (PTI-428), sodium 4-phenylbutarate (4PBA), SION-638, SION-109, SION-451, SION-719, VRT-532, N6022, or combinations thereof.
